(19) United States
(12) Patent Application Publication
(10) Pub. No.: US 2025/0049488 A1
(43) Pub. Date: Feb. 13, 2025

(54) DEVICES AND METHODS FOR BONE FIXATION

(71) Applicant: RevBio, Inc., Lowell, MA (US)

(72) Inventors: Brian J. Hess, Charlestown, MA (US); George W. Kay, Sharon, MA (US); Andrey Marchenko, Acton, MA (US); Sourabh Boruah, Lowell, MA (US); Michael C. Brown, Harvard, MA (US); Bethany Silva, Wrentham, MA (US); Alan Pollack, Hastings On Hudson, NY (US)

(21) Appl. No.: 18/717,801

(22) PCT Filed: Dec. 15, 2022
(86) PCT No.: PCT/US2022/053088

§ 371 Date: Jun. 7, 2024

Related U.S. Application Data
(60) Provisional application No. 63/289,927, filed on Dec. 15, 2021.
(60) Provisional application No. 63/411,408, filed on Sep. 29, 2022.

(51) Int. Cl. A61B 17/88; A61B 17/56

(52) U.S. Cl. CPC A61B 17/8866; A61B 2017/564

(57) ABSTRACT

Described herein are devices and methods for fixating bone segments, e.g., including the intra-operative or provisional positioning, retention, orientation, or fixation of bone segments to allow injection or placement of an adhesive composition into or around the kerf or space between bone segments until said adhesive composition solidifies or cures.

### CLAIM OF PRIORITY

This application claims priority to U.S. Provisional Application No. 63/289,927, filed on Dec. 15, 2021, and U.S. Provisional Application No. 63/411,408, filed on Sep. 29, 2022. The entire contents of each of the foregoing applications is hereby incorporated by reference in their entirety.

### BACKGROUND

Current devices and methods of use related to bone repair following fractured or cut bone often require hardware fixation devices (e.g., plates, clips, wedges, screws, pins, nails, wires, rods, or anchors) to be installed to enable the bone segments to be positioned, orientated, stabilized, retained, or fixated in order to undergo fracture healing. Often patients are treated following traumatic or pathological bone injury or as a result of a surgical intervention to correct a bone deformity, replace a joint, or to gain access to treat some other condition or disease.

Typically, these devices for bone repair are left permanently installed or require a secondary surgical intervention for removal. Often these devices span across and outside the kerf or space between the bone segments, and as a result of their placement position, the devices protrude outside the contour of the bone surface. Due to this placement, the presence of these devices can be felt by the subject, may be visible through the soft tissue envelope, or can cause soft tissue irritation or pain over time from tendons, ligaments, or nerves running adjacent to the bone surfaces and in contact with these devices. These devices are typically left in place for the life of the subject unless a secondary surgery is indicated to remove them. These practices often leave the healing or repaired region susceptible to complications (e.g., implant loosening, infection, site irritation or pain) or often cause possibly delayed or incomplete fracture healing (e.g., malunion).

As such, there is a need for devices and methods for bone fixation which alleviate many of the complications common in long-term fixation device use without requiring secondary surgeries for removal.

### SUMMARY

The current disclosure features, inter alia, a device that can position bone segments, bone fragments, or other structures of any shape, size, and/or location in position and related methods, e.g., preventing the movement of said bone segments, e.g., during a surgical procedure. In some embodiments, bone segments consist of a length of bone which may be part of a whole or may be a cut off segment, e.g., cut off in a surgical setting. In some embodiments, bone fragments consist of a broken off part of a bone. In some embodiments, said other structures include one or more of the following: a bone fragment preoperatively present at the site, e.g., a proximal segment, a distal segment, an unattached fragment of bone, an adjacent bone, or some similar such bone part; a synthetic implantable object, e.g., metallic object; a ceramic object; a synthetic object comprising regenerative material; a biologically generated implantable object, e.g., autogenous bone, allograft block bone, xenograft block bone, block of coral-generated graft material, or otherwise processed, e.g., demineralized, freeze-dried, frozen, irradiated, ion-exchanged; a biologically generated tissue or material; or a structure additively manufactured with synthetic materials or bioprinted in combination with cellular elements. In some embodiments, the devices described herein are used to align and fixate two or more bone segments, bone fragments or other structures in a desired position. In some embodiments, the devices described herein comprise a means for delivery of an adhesive composition to partially or fully fill the kerf or gap space between or around bone segments or other implantable structures. In some embodiments, the device is used to prevent movement of bone segments or other structures during delivery of a composition, e.g., an adhesive composition to the application site and curing of said composition between said bone segments, thus maintaining desired alignment and enabling fixation.

In some embodiments, the device is used to prevent movement of bone segments, bone fragments or other structures during fixation using external fixation hardware, thus maintaining a desired bone alignment. In some embodiments, said external fixation hardware is metallic or some other such biologically compatible material. In some embodiments, the fixation hardware is presented as a feature of the devices described herein. In some embodiments, the fixation hardware is presented as a separate component. In some embodiments, the device is removed (e.g., partially or totally removed) during the procedure, e.g., after the adhesive composition has cured or the external fixation hardware has been installed. Said external fixation hardware may take many forms (e.g., plates, clips, splints, wedges, screws, pins, nails, wires, rods, or anchors) and may be used with various bone types (e.g., cranial, facial, sternum, ribs, vertebrae, innominate bone, arm bones, and leg bones). In some embodiments, the bone segment to be fixated is a long bone, e.g., a mandible, metacarpal or tibia. In some embodiments, the bone segment to be fixated is a flat bone, e.g., a sternum. In some embodiments, dimensional proportions of the device reflect the shape and size of the bone subject to fixation. In some embodiments, fixation hardware is removed at the end of the procedure, e.g., after an adhesive composition has been delivered and cured. In some embodiments, fixation hardware remains at the fixation site post-operatively, e.g., for a number of hours, days, weeks, months or years.

The devices described herein may be used with bone segments created surgically, e.g., a sternotomy, or used on deformed, fractured or broken bones, or on other biocompatible structures. In some embodiments, the device is used to hold and align bone segments together during a surgical procedure or to provide proper spacing for delivery of an adhesive composition or bone grafting material, other construct or other user-selected compositions. In some embodiments, use of a device as described herein enables the use of an adhesive composition to stabilize and fixate bone segments, reduce complications following surgery, or improve fracture healing results compared to the use of existing standards of care (e.g., long-term hardware fixation devices).

In some embodiments, the devices described herein allow for introduction of an adhesive composition into or onto the surgical site by means of a single injection, multiple injections, or other application methods. In some embodiments, the adhesive composition fixates the bone segments or other structures and optionally eliminates the need for leaving external fixation hardware installed, e.g., which are either permanent or require a subsequent surgical intervention to remove. In some embodiments, the devices described herein allow a user to repeatedly position or realign and temporarily fixate bone segments in a static position, separate from each other, to allow filling of the kerf or space between the bone segments with an adhesive composition, wherein the bone segments are then repositioned to their natural or otherwise desired anatomical alignment. In some embodiments, the device comprises a mechanical feature that bonds, attaches to or grasps a bone segment or other structure to be fixated in one or more locations using a mechanical connection (e.g., screws, rods, plates, nails, wires, or other similar such mechanism) without the mechanical feature fitting within the kerf space.

In some embodiments, the devices described herein create a gap space between the bone segments to be fixated such that an adhesive composition can be delivered between them. In some embodiments, the device is used to grasp each bone segment and separates the segments to create a gap, and then reposition the bone segments into a preferred anatomical alignment once the fixation mechanism (e.g., fixation hardware or adhesive composition) has been delivered or installed. In some embodiments, said devices are then used to close the gap such that the bone segments to be fixated are moved back to the preselected position that they were in prior to the device creating the gap. In some embodiments, said devices are then used to partially close the gap such that the bone segments to be fixated are moved back into the preselected position that they were in prior to the device use in creating the gap. Such capabilities may enable a sufficient amount of an adhesive composition to be delivered to fixate bone segments wherein the original placement may not have provided enough space for sufficient adhesive composition. In some embodiments, the devices described herein are comprised of a biocompatible material, e.g., a naturally occurring or non-naturally occurring (e.g., synthetic) material. In some embodiments, a gap created by a device between one or more bone segments positioned, e.g., as described herein, is filled with a solid object or implant that is fixated to the bone with an adhesive composition. In some embodiments, the solid object or implant includes a metallic, i.e., titanium, implant or be a bone substitute formed from a solidified form of the adhesive composition. In further embodiments, the shape and size of the solid object or implant is sized to fit or match the profile or outer contour of the gap. In some embodiments, the solid object or implant can withstand stress, e.g., tensile, shear, bending, or torsion, of least 100 kPa.

The devices described herein may be removed prior to the end of the surgical procedure or may be retained at the surgical site for an extended length of time (e.g., minutes, hours, days, weeks, months, years after application, or indefinitely). In some embodiments, the devices described herein range in overall size from about 10 mm to about 1500 mm, e.g., about 10 mm to about 50 mm, about 50 mm to about 150 mm, about 150 mm to about 300 mm, about 300 mm to about 500 mm, about 500 mm to about 1000 mm, or about 1000 mm to about 1500 mm. In some embodiments, the bone segments to be fixated may be of any size, e.g., from about 1 mm to about 500 mm, e.g., about 1 mm to about 10 mm, about 10 mm to about 30 mm, about 50 mm to about 100 mm, or about 250 mm to about 500 mm. The size of the bone segment to be fixated may determine the size of the device to be used.

In some embodiments, an adhesive composition is provided which enables fixation of bone segments and kerf sealing through application of said adhesive composition between the bone segments and within the contour of the external bone surface. As a result, the fixation may not be felt or seen through the skin once the temporary fixation devices have been removed and may not cause soft tissue irritation or pain from tendons, ligaments, or nerves running adjacent to the bone surfaces since the adhesive composition is inline or within the contour of the fixated bone segments or the bone in its entirety.

The present disclosure provides that the device embodiments described herein may utilize one or more mechanical features for grasping, positioning, aligning and fixating the bone segments in a preferred position (e.g., the original position of the bone segment, or an alternate position). In some embodiments, the device utilizes mechanical features such as screws, pins, plates, protrusions, clamps, clips, wedges, or additional supportive mechanical features. In some embodiments, said mechanical features comprise a biocompatible material. In some embodiments, the device comprises a mechanical feature that fits within the dimensions of the kerf or space between the bone segments. The mechanical feature that fits within the kerf or space between the bone segments may comprise a feature that bonds or attaches to a bone in one or more locations using screws, rods, plates, suction, tines, wires or elastic retaining element to provide central support and adjustability for positioning the bone segments into desired position or relationship and to allow for repeated repositioning or alignment of the bone segments throughout the procedure.

In some embodiments, said mechanical feature is removed once an adhesive composition is delivered. In some embodiments, the mechanical feature is maintained in position until the adhesive composition has cured. Said mechanical feature may comprise a biocompatible material, e.g., a naturally occurring or non-naturally occurring (e.g., synthetic) material. Exemplary biocompatible materials may include, but are not limited to, steel or steel alloys; titanium or titanium alloys; polymer materials such as polyether ether ketone (PEEK), Polycarbonate, polyethylene terephthalate (PET), polyethylene (PE), polypropylene; xenograft; allograft; bone substitute; or similar such materials and combinations thereof. In some embodiments, the devices of the present disclosure may comprise a resorbable biocompatible material component that is dispersed. In some embodiments, the resorbable component is detachable from the device. In some embodiments, the resorbable biocompatible material comprises a solidified form of the adhesive composition that is formed prior to implantation. In some embodiments, the resorbable biocompatible material is replaced with host tissue over time after implantation. In some embodiments, the device comprises a non-resorbable material, such as a metal, plastic, or other non-bone material.

In some embodiments, the device is of sufficient strength to withstand placement forces or retention of its position during use from opposing forces acting to separate the bone segments (e.g., muscle or soft tissue retraction forces) while the adhesive composition solidifies or cures. In some embodiments, the device is of sufficient size and/or strength to withstand forces required for hardware fixation application. In some embodiments, the device is of sufficient size or strength to withstand adjustment and removal forces. In some embodiments, the device comprises a malleable material or feature, e.g., to allow adjustment of bone segments into alignment, orientation or into a desired position. The present disclosure provides that the devices described herein may include a feature to lock or prevent movement of the bone segments to be fixated on all but one axis of rotation while the device is in use. In some embodiments, said devices prevent movement of bone segments to be fixated via a hinged mechanism. In some embodiments, said devices prevent movement of bone segments to be fixated via a sliding mechanism. In some embodiments, the devices described herein selectively move one, several, or each bone segment along the translational axis to enable delivery of an adhesive composition between the selected bone segments while maintaining their spatial position at a treatment site, e.g., a surgical site. In some embodiments, the devices described herein are used to create or widen a gap space between the bone segments to be fixated such that an adhesive composition can be delivered there between. In some embodiments, said devices are used to narrow or close the gap such that the bone segments to be fixated are moved back to the position they were in prior to the creation or opening of the gap. Such capability may enable a sufficient amount of an adhesive composition to be delivered to fixate bone segments where the original placement may not have provided enough space for the placement of sufficient amount of adhesive composition. In some embodiments, the device is used to facilitate stabilization of bones, bone segments, or other devices.

The present disclosure features a device which includes a rail disposed substantially parallel to the one or more bone segments or bone fragments to be positioned or fixated. In some embodiments, the rail fixation device further comprises a plurality of base elements slidably coupled to and disposed along a length of the rail. In some embodiments, the device further comprises a plurality of retaining elements, each having a first end operatively coupled to one of the plurality of base elements and a second end constructed and arranged to hold at least one of the one or more bone segments or bone fragments. In some embodiments, the one or more retaining elements comprise a grasping mechanism which further comprises tines.

In some embodiments, the connection between each of the plurality of base elements and the first ends of the plurality of retaining elements permits up to three degrees of rotational freedom and up to three degrees of translational freedom of each of the plurality of retaining elements relative to the rail. The connection between each of the plurality of base elements and the first ends of the plurality of retaining elements may be securable following adjustment along one or more rotational and/or translational degrees of freedom, i.e., securable mechanically, e.g., a reversible locking mechanism, or chemically, e.g., an irreversible cementation. In some embodiments, a position of each of the plurality of base elements along the rail is capable of being secured mechanically, e.g., via a reversible locking mechanism.

In some embodiments, the connection between each of the plurality of base elements and the first ends of the plurality of retaining elements may include a hinge, a telescopic member, or ball-and-socket connection. In some embodiments, the base elements of the device include a protrusion adapted to connect to an external support, e.g., a tabletop support, i.e., a bracing element. In some embodiments, the rail feature of the device includes one or more stops along its length arranged to limit translation of the plurality of base elements.

In some embodiments, the rail is curved and ensures motion of bone fragments and other structures along a curved path to allow motion of bone fragments along specific contours or to expand the available space in the surgical field in specific preferred manner. In some embodiments, the rail may be straight. In some embodiments, the rail may be comprised of several independently moveable segments to allow for a combination of straight and curved elements to ensure mimicry of the natural contours of the bone segments being fixated and/or to expand the available space in the surgical field. In some embodiments, the rail comprises offsets in the translational movement continuity. In some embodiments, the rail comprises a plurality of segments which are intended for base element attachment and segments which are not intended for base element attachment. In some embodiments, the rail comprises structural features allowing attachment of bracing elements which provide support to the device construct. In some embodiments, the rail is between 10 mm and 1500 mm in length and between 1 mm and 200 mm in width.

In another aspect, the present disclosure features a device for positioning or fixating one or more bone segments or bone fragments, wherein said device comprises a base element comprising one or more arms connected by a hinge. In some embodiments, the hinged device further comprises one or more retaining elements, each having a first end slidably coupled along a length of one of the one or more arms and a second end constructed and arranged to hold at least one of the one or more bone segments, e.g., bone fragments, or other structure to be fixated.

In some embodiments, each of the plurality of retaining elements includes one or more arms. In some embodiments, each of the plurality of retaining elements comprises a surface capable of supporting bone segments, e.g., a concave surface, e.g., an approximately cylindrical conical or spherical concave surface; a flat surface; or a convex surface. In some embodiments, each of the plurality of retaining elements may comprise one or more contact points capable of supporting or holding one or more bone segments, bone fragments, or other structure. In some embodiments, each of the one or more contact points comprises one or more tines or similar such grasping mechanism in opposition. In some embodiments, the one or more contact points is disposed orthogonally to the one or more arms of the plurality of retaining elements. In some embodiments, the one or more contact points are hinged to accommodate bone segments or other structures of non-uniform shape. In some embodiments, the one or more opposing contact points are aligned to minimize bending of the retained bone segment(s) or structure(s). In some embodiments, the contact points are positioned on elongated and slender tines for transcutaneous and minimally invasive applications (e.g., transcutaneous or closed procedures). In some embodiments, the contact points consist of one or two tines which extend from the device to support the bone segment, bone fragment or other structure. In some embodiments, the hinged device includes a bracing element constructed and arranged to connect the device to an external support (e.g., a tabletop support).

In another aspect, the present disclosure features a method of fixating one or more bone segments or bone fragments or other structures at a location, e.g., a surgical site. In some embodiments, the method includes using the device to grasp each bone segment or other structure to be fixated sequentially, e.g., via the grasping portion, e.g., tines, of the retaining element. The method may include a step of attaching the one or more bone segments or bone fragments to one or more retaining elements of a fixation device. In some embodiments, the method further includes positioning the one or more bone segments or other structure in a preferred spatial relation or position relative each other and the fixation device. In some embodiments, the method further includes a step of securing the position, e.g., reversibly locking one or more, or all of the degrees of freedom of relative motion among the one or more bone fragments, segments, or structures, into the preferred spatial relationship. In some embodiments, the method further includes the step of releasing the lock on one or more of the degrees of freedom of motion in order to open a space or a gap along a rotational or translational path. In some embodiments the method allows this movement for the placement an adhesive. In some embodiments the movement of opening the space or gap is reversed to reproduce the original spatial arrangement of the structures. In other embodiments, this method allows for the placement of other means of fixation, e.g., hardware fixation, e.g., places, pins, nails, etc., which may be at the time intended to provide intraoperative temporary fixation or intended for extended or permanent use. The hardware devices may themselves be retained by adhesive composition or screws, or both.

In another aspect, the present disclosure features a device useful in procedures treating discontinuities of a bone, e.g., following trauma resulting in bone fracture, resection of bone segment, or discontinuity resulting from a procedure intended to treat a deformity. According to one embodiment, the present disclosure features a device useful in procedures treating discontinuities of a bone resulting from resection of bone volume, either alone or together with a bone graft or a bone substitute. Regeneration of resected volume and continuity bone might be attempted following resection of affected bone for such conditions and diseases as bone cancer, e.g., osteosarcoma, bone infections, e.g., osteomyelitis, or another disease, e.g., fibrous dysplasia, Paget's disease, or other similar conditions.

According to another embodiment, the present disclosure features a device useful in establishing new spatial relationship between one and another segment of a bone, e.g., distal and proximal ends of a long bone. This utility might be applied during procedures aimed at changing the shape or size of a bone, e.g., correcting a deformity of a bone resulting from a condition or a disease, e.g., rickets, osteogenesis imperfecta, etc.; procedures aiming to lengthen a limb bone, e.g., leg or arm, or another bone, e.g., mandibular sagittal split procedure; or procedures aimed at shortening or rearranging the spatial relationship between parts of bones, e.g., Le Fort osteotomies. In another aspect, the present disclosure features a device useful in performing fusion of adjacent bones, i.e., arthrodesis, by immobilizing adjacent bones, e.g., tarsals, carpals, vertebrae, etc., in a favorable spatial relationship for their fusion. The immobilization and stabilization may be performed intraoperatively and temporarily while other means of fixation, e.g., an adhesive composition, hardware, etc., are applied and deemed effective.

In some embodiments, the devices described herein can aid in procedures involving fixation of parts of long bones (e.g., humerus, radius, ulna, femur, tibia, fibula, metacarpals, metatarsals, and phalanges). In some embodiments, the devices described herein can aid in procedures involving fixation of parts of flat bones (e.g., sternum, scapula, and clavicle). In some embodiments, the devices described herein can aid in procedures involving fixation of parts of craniofacial bones (e.g., mandible, maxilla, and zygoma). In some embodiments, the devices described herein can aid in procedures involving fixation of parts of other bones (e.g., pelvis, ribs, vertebrae, and other bones the fragments of which can be extracted and later realigned).

In some embodiments, the devices described herein can aid in procedures involving fixation of adjacent bones relative one another, e.g., fusion at the ankle, wrist, etc., used to treat arthritic conditions, e.g., osteoarthritis, of joints, i.e., arthrodesis. In such cases there may be two, three, or more individual bones or other structures, e.g., bone plates, engaged, positioned, aligned immobilized, or held by the device. The device may remain in function while other means of stabilization, e.g., adhesive composition, screws, pins, clips, etc., are applied and become effective.

In some embodiments, the methods of use of the device described herein rely on the device substantially controlling all six degrees of freedom of relative motion, i.e., three translational and three rotational, of each structure engaged by the device. In some embodiments, the methods of use of the device described herein rely on the device substantially controlling several, but not all six, i.e., five, four, three, or fewer, degrees of freedom of relative motion, i.e., three translational and three rotational, of each structure engaged by the device, as some of the degrees of motion are limited by the mechanical context, i.e., other structures e.g., tendons, ligaments, muscles, bones, etc., substantially limiting movements afforded by the degrees of freedom not limited by the device described herein. For example, the device might be used to translationally distract the structures, e.g., bone fragments or segments, from one another without the need to rotationally stabilize them because they are rotationally stabilized by surrounding investing tissues. In such a case the retaining elements used can be structurally simpler and need not possess features specifically designed to limit the degrees of freedom already limited by the anatomical context. For example, the embodiment of the device used in this method may not require tines to grasp the structure and limit its rotational movements, as a retaining element may only require presenting a single unopposed surface engaging each structure to apply the distracting force to accomplish the task.

The range of applications of the devices described herein may include clinical presentations in which a gap in bone exists which can be either filled with an adhesive composition, e.g., as described herein, which solidifies in situ, or with a solid form, e.g., preformed bone substitute or regenerative biomaterial, which can be adhered into the gap and may improve stable alignment of the bone fragments postoperatively. The range of applications of the devices described herein may include clinical presentations in which a gap in bone is surgically created. The range of applications of the devices described herein may include clinical presentations in which a gap in bone is surgically created to treat a condition of ill-formed or incompletely formed bone segments, e.g., deficient height, length or width of a bone segment, which can be either filled with an adhesive composition, e.g., as described herein, which solidifies in situ, or with a solid form, e.g., preformed bone substitute or regenerative biomaterial, which can be adhered or otherwise fixated into the gap and may improve stable alignment of the bone fragments postoperatively. In some embodiments, the subject is a child. In some embodiments, the subject is an adult. In some embodiments, the subject is a senior (e.g., an adult over the age of 65) or in a decline of the skeletal state. In some embodiments, the subject is a human or a non-human vertebrate, e.g., a canine, equine, feline, etc. In some embodiments, said devices, compositions and methods of use thereof are intended for use in a surgical intraoperative environment, to fixate or immobilize traumatic injuries in a field environment, or during transport of a patient.

In another aspect, the present disclosure features a kit including a fixation device as described herein, for fixating a position of one or more bone segments, bone fragments or other structures, and an adhesive composition, e.g., as described herein, comprising a multivalent metal salt, an organic compound, and an aqueous medium, and optionally, an additive.

In another aspect, the present disclosure features a kit including one or more retaining elements, e.g., as described herein, connectable to and detachable from a device as described herein, for fixating one or more bone segments in a desired spatial relationship and an adhesive composition, e.g., as described herein, which consists of a multivalent metal salt, an organic compound, and an aqueous medium.

In another aspect, the present disclosure provides a device for positioning or fixating one or more structures. In some embodiments, the device includes a first rotary hinge-type linkage arm including a hinge and one or more retaining elements, wherein this linkage arm enables movement of the attached bone segment or bone fragment in only one direction, e.g., one degree of freedom. In some embodiments, the device further comprises a second linkage arm consisting of a twin ball joint-type linkage arm including at least one ball joint and one or more retaining elements, wherein this linkage arm enables movement of the attached bone segment or bone fragment in up to three degrees of freedom. In some embodiments, each of the first and second linkage arms further comprise a mechanical means for reversibly locking the position of the attached bone segment or bone fragment, e.g., a knob. In some embodiments, the first and second linkage arms are constructed and arranged to be independently positioned to hold at least one of the one or more structures. Additional features of the present disclosure and the devices, compositions, and methods of use thereof are described in greater detail in the Detailed Description, Figures, and Claims, below.

### BRIEF DESCRIPTION OF THE FIGURES

FIGS. 1A-1C depict a positioning device according to an embodiment of this disclosure in use with models of fractured bone segments, wherein A refers to the arm of the device, CP refers to the contact points of the device, RE refers to the retaining elements, B refers to the base elements, and R refers to the rail of the device. FIG. 1A depicts a positioning device in use grasping two model pieces depicting bone fragments prior to aligning the segments. FIG. 1B depicts a positioning device in use aligning the model bone fragments. FIG. 1C depicts a positioning device in use compressing the model bone fragments in a preferred alignment together with the aid of an elastic band.

FIGS. 2A-2C depict another positioning device according to an embodiment of this disclosure in use with models of fractured bone segments, wherein A refers to the Arm of the device, CP refers to the Contact Points of the device, RE refers to the Retaining Elements, and H refers to the Hinge of the device. FIG. 2A depicts a positioning device in use grasping two model pieces depicting fractured bone segments prior to aligning the segments. FIG. 2B depicts a positioning device in use aligning the model bone segments. FIG. 2C depicts a positioning device in use compressing the model bone segments in a preferred alignment by changing the angle of H.

FIGS. 3A-3D depict positioning devices according to an embodiment of this disclosure in use fixating a standalone fractured long bone. FIG. 3A depicts three positioning devices attached via their base elements B to a rail R, prior to grasping the fractured bone segments. FIG. 3B depicts the positioning devices with arms A extended and contact points CP grasping the fractured bone segments. FIG. 3C depicts the positioning devices aligning the bone segments and separating them along the translational axis so that a gap exists between each segment, but their spatial relationship is still maintained. FIG. 3D depicts the positioning devices moving the bone segments back to their anatomical positions.

FIGS. 4A-4E depict positioning devices according to an embodiment of this disclosure in use fixating a fractured cadaverous long bone. FIG. 4A depicts the fractured long bone, with arrows pointing to the fractured bone segments. FIG. 4B depicts three positioning devices in use grasping the fractured bone segments. FIG. 4C depicts the user adjusting the positioning devices to align the bone segments into their appropriate spatial relationship. FIG. 4D depicts an adhesive composition being delivered to the gap created between each bone segment. FIG. 4E depicts the positioning devices grasping the bone segments while the adhesive composition cures, and shows the rail R placed in parallel to the long bone.

FIGS. 5A-5C depict a retaining element RE component of a positioning device according to an embodiment of this disclosure from various angles, and its individual components. FIG. 5A depicts a RE from a lateral view with components labelled, wherein A refers to the Arms, S refers to the stop, which in this case is a screw lock mechanism, H refers to the hinge component, and CP refers to the Contact Points. FIG. 5B depicts a RE from an angled top-down view with the components labelled. FIG. 5C depicts a RE from a top-down view with the components labeled.

FIGS. 6A-6B depict positioning devices according to an embodiment of this disclosure in a computer model grasping a model mandible, with the components of the device labelled. FIG. 6A depict positioning devices according to an embodiment of this disclosure grasping the model mandible in an extended position, wherein CP refers to the Contact Points, H refers to the Hinges, A refers to the Arms, RE refers to the Retaining Elements, R refers to the Rail, B refers to the bases, and C refers to the Connectors. FIG. 6B depict positioning devices according to an embodiment of this disclosure grasping the model mandible in a compressed position.

FIG. 7A depicts a positioning device including two rotary hinge-type linkage arms RHLA permitting 1 degree of freedom and three twin ball joint-type linkage arms TBLA permitting 6 degrees of freedom. Each set of linkage arms LA further comprises a knob K for enabling and locking motion of the associated set of linkage arms LA FIG. 7B such a device in use grasping bone segments of a canine mandible to be aligned and fixated.

FIGS. 8A-8R depict a positioning device including a rotary hinge-type linkage arm RHLA set and a twin ball joint-type linkage arm TBLA set in use to temporarily fix the position of bone segments of a canine mandible.

FIG. 8A depicts the bone segments of the canine mandible to be fixed.

FIGS. 8B and 8C depict the attachment of the bone segments of the canine mandible to each of the rotary hinge-type linkage arm RHLA (FIG. 8B) and the twin ball joint-type linkage arm TBLA (FIG. 8C) of the fixation device.

FIGS. 8D-8F depict using the fixation device to manipulate the bone segments of a canine mandible in various positions.

FIGS. 8G and 8H depict placement of the bone segments at a desired position (FIG. 8G) and locking the twin ball joint-type linkage arm set TBLA (FIG. 8H) such that the bone segments of the canine mandible maintain their position.

FIG. 8I depicts the marking and drilling of holes into the bone segments of the canine mandible for accepting a permanent fixation mechanism to be attached, such as a bone plate.

FIGS. 8J-8L depict the manipulation of the rotary hinge-type linkage arm RHLA to adjust the compressive reduction and distention of the fracture between the bone segments of the canine mandible through use of a piece of external fixation hardware.

FIG. 8M depicts the application of an adhesive composition to the proximal ends of the bone segments of the canine mandible.

FIGS. 8N and 8O depict the repositioning of the bone segments of the canine mandible following application of the adhesive.

FIG. 8P depicts the removal of excess adhesive from the position-locked bone segments of the canine mandible.

FIG. 8Q depicts the securement of the external fixation hardware, e.g., using screws, to the bone segments of the canine mandible.

FIG. 8R depicts the stabilization of the fractured bone segments of the canine mandible until the applied adhesive has cured.

FIG. 9A depicts a retaining element RE including three contact points CP, e.g., tines T, suitable for limited access space and further including a two-tined jaw TTJ and an opposing one-tined jaw OTJ.

FIG. 9B depicts a retaining element RE including four contact points CP suitable for weaker bone fragments where failure through bending is a concern, further including a two-tined jaw TTJ and an opposing hinged two-tined jaw HTJ.

FIG. 10A depicts a positioning device with elongated retaining elements RE1 and RE2 which allows expanded surgical access at the fracture or defect location, and including an articulating locking mechanism L which allows up to three degrees of freedom for aligning and positioning bone fragments and one degree of freedom for fracture or defect distention and compression.

FIG. 10B depicts a top view of the retaining element RE of FIG. 10A showing the opposing retaining elements RE1 and RE2.

FIG. 10C depicts the rail fixation device RFD shown in FIG. 10A further including detachable handles H1 and H2 on the retaining elements RE1 and RE2, thus allowing further access for the user.

FIG. 11 depicts a positioning device according to an embodiment of this disclosure for use in positioning bone segments, where RE refers to the retaining elements, B refers to the base elements, and R refers to the curved rail of the device.

### DETAILED DESCRIPTION

The present disclosure features, inter alia, devices for fixating bone segments of any shape, size, and/or location in position and an adhesive composition, as well as methods of using the same. Components and embodiments of the devices, compositions, and methods of use thereof are described herein in greater detail. Aspects of one embodiment may be found in all embodiments, or the devices may be used in concert, even if not explicitly described.

As used herein, the term “device” refers to a mechanical apparatus used to fixate one or more bone segments, bone fragments, or other structures, e.g., in position. In an embodiment, the device fixates a bone segment or a plurality of bone segments so that an adhesive composition (e.g., an adhesive composition described herein) may be delivered to the space around the bone segments or the plurality of bone segments, such as the kerf or other space between or around the bone segments. A device may be made of any material, such as a metallic or polymeric material. Exemplary devices are described herein.

As used herein, the term “kerf” refers to a cut line or gap in a tissue. In an embodiment, a kerf is created by a surgical procedure, e.g., an osteotomy (e.g., a sternotomy).

As used herein, the term “bone fragment” refers to a shortened piece of bone, such as a piece of bone that has broken off from a larger bone.

As used herein, the term “bone segment” refers to a portion or length of bone which may be part of a whole or a portion which has been cut off.

As used herein, the term “structure” refers to any object, e.g., bone fragment, bone segment, adjacent bone, implantable device, or other biologically compatible segment that mimics bone, which is subject to being grasped, stabilized, retained, positioned, fixated or held.

### Devices

The present disclosure provides that embodiments of a device that permit intraoperative or provisional fixation of two or more structures, e.g., bone segments, bone fragments, bones or implants, or other structures. In some embodiments, said devices provide temporary immobilization of the bone segments, bone fragments, or other structures during delivery of a means of permanent fixation, e.g., an adhesive composition or bone fixation hardware, e.g., pin, nail, plate, K-wire, etc. In some embodiments, the structure is a bone. In some embodiments, the structure is a bone segment or bone fragment, preoperatively present at the site, e.g., proximal segment, distal segment, unattached fragment of bone, etc. In some embodiments, the structure is a synthetic implantable object, e.g., a metallic object, regenerative material, or the like. In some embodiments, the structure is a biologically generated implantable object, e.g., autogenous bone, allograft block bone, xenograft block bone, block of coral-generated graft material, or other similar structures. In some embodiments, the devices described herein are constructed and arranged to reproducibly and repeatably position bone segments, bone fragments or other structures. In some embodiments, device embodiments disclosed herein permit delivery of an adhesive composition by providing temporary immobilization of the bone segments, bone fragments, or other structures to be fixated. In some embodiments, devices disclosed herein enable delivery of an adhesive composition by electively separating the structures, e.g., bone segments or fragments, to be fixated and then compressing the bone segments together or reducing the gap width once the adhesive composition has been delivered, to enable permanent or definitive fixation of the bone segments. This elective separation and compression of bone segments to be fixated may be achieved mechanically, e.g., via hinges or joints, or via physical forces, e.g., elastic force or compression force. In some embodiments, the relative movement of the structures engaged by the device is reversible by virtue of the construction of the device as described herein, e.g., by locking and unlocking a mechanical feature of the device, e.g., a locking knob. In some embodiments, the relative movement of the structures engaged by the device is reproducible by virtue of the construction of the device as described herein. In some embodiments, the relative movement of the structures engaged by the device is repeatable by virtue of the construction of the device as described herein.

In an aspect, the present disclosure features a device comprising a biocompatible material, a naturally occurring or non-naturally occurring, e.g., synthetic, material. For example, said biocompatible material may comprise one or more of steel or steel alloys; titanium or titanium alloys; plastic materials such as polyether ether ketone (PEEK), polycarbonate, polyethylene terephthalate (PET), polyethylene (PE), polypropylene, xenograft, allograft, similar such materials and combinations thereof. In some embodiments, said devices comprise a resorbable biocompatible material component that disperses, e.g., a solidified form of an adhesive composition. In some embodiments, the resorbable biocompatible material component is detachable from the device. In some embodiments, said resorbable biocompatible material is replaced with host tissue over time. In some embodiments, a device may be removed after intraoperative use, i.e., once an adhesive composition is delivered and has sufficient strength for fixation of bone segments. In some embodiments, an adhesive composition is delivered within the kerf or space between bone segments and the cured adhesive composition does not protrude above or below the kerf line. In some embodiments, the devices described herein may remain installed, therefore lowering the risk of infection.

The present disclosure features devices and methods of use of said devices and an adhesive composition to provide fixation of bone segments by application of the adhesive composition between the bone segments. In some embodiments, the devices act as a temporary fixation mechanism during delivery and curing of an adhesive composition. In some embodiments, the devices are removed intraoperatively once the delivered adhesive composition has cured. As a result, the fixation of the bone segments may not be felt or seen through the skin and may reduce soft tissue irritation or pain from tendons, ligaments, or nerves running adjacent to the bone surfaces as the adhesive composition is inline or within the contour of the fixated bone segments.

In some embodiments, a device described herein includes one or more radiopaque or radiolucent materials. In some embodiments, the adhesive composition, e.g., as described herein, includes a radiopaque additive. In some embodiments, when the device is composed of a radiolucent material and the adhesive composition to be delivered comprises a radiopaque additive, said device does not interfere with imaging of the fracture site, e.g., fluoroscopic imaging or x-ray imaging, and only the radiopaque adhesive composition will imaged. The device embodiments described herein may be comprised of a metal, e.g., titanium, a TiAlV alloy, stainless steel, or aluminum, a crystalline or non-crystalline ceramic, e.g., alumina, zirconia, lithium disilicate, felspathic glass, or a polymeric material. In some embodiments, a device described herein can include a stiffening material, e.g., carbon fiber, glass fiber, aramid fiber, or a ceramic fiber. In some embodiments, individual device components are comprised of the same material or of varying materials.

In some embodiments, the fracture to be fixated is non-comminuted and non-gapped. In other embodiments, the fracture to be fixated is a simple fracture, a complex fracture, a gapped fracture, a comminuted fracture, or a similar condition requiring the distraction of two or more bone segments.

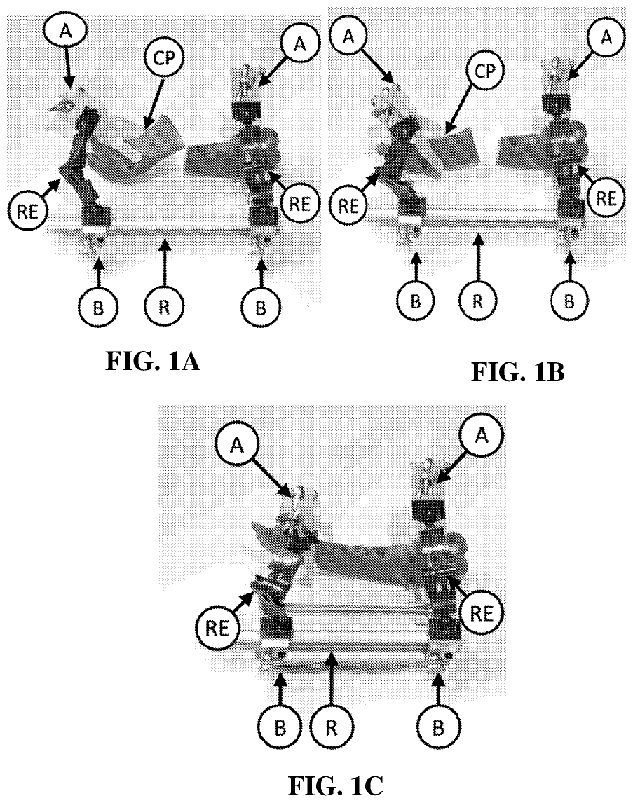

In an embodiment, the present disclosure features a rail fixation device (RFD), such as illustrated in FIGS. 1A-1C. An RFD may be used, for example, in the fixation of long bones. In some embodiments, an RFD includes at least the following components: a rail R, one or more retaining elements RE, and one or more base elements B. In some embodiments, the base elements B are slidably coupled to and disposed along a length of the rail such that they can be translated along the rail and secured to a position on the rail. In some embodiments, a rail fixation device RFD comprises additional elements, such as additional retaining elements REs, stops S, one or more retaining element connectors C, one or more arms A and, extending from said arms A, tines T for grasping the bone segment, bone fragment or other structure to be fixated. In some embodiments, the rail component R of the device is rigid, i.e., resistant to torsional and bending stresses. In some embodiments, R is rectilinear.

In some embodiments, such as illustrated in FIG. 11, an RFD can include one or more retaining elements RE (illustrated as black dashed line boxes), one or more base elements B, and a curved rail R. As illustrated, the base elements B can include a locking point L₁ that can be used to secure the position of the base elements B at a position on the curved rail R. Connected to the base elements B are linkage arms LA, here illustrated as a single ball joint whose position is reversibly lockable using locking point L₂. The retaining elements RE, illustrated as black dashed line boxes, can be any suitable retaining element as described herein. Operationally, the rail-based RFD operates substantially the same as the linear rail RFD embodiment illustrated in FIGS. 1A-1C as it generally includes the same or similar types of the one or more retaining elements RE and one or more base elements B. The embodiment of an RFD shown in FIG. 11 is just one embodiment, and the specific structural features illustrated in FIG. 11 in no way limit the scope of this disclosure. The retaining elements RE, one or more base elements B, linkage arms LA, and locking points L₁ and L₂ may be any suitable structure as disclosed herein. In some embodiments, R comprises a curved surface, or an offset. In some embodiments, rail R lies substantially parallel to the long axis of the bone segments or structures(s) to be fixated, i.e., disposed substantially parallel to the one or more bone segments or bone fragments to be positioned or fixated when the device is in use. In some embodiments, R lies at an angle to the surface of interest, e.g., approximately perpendicular to the surface of the bone defect. In some embodiments, rail R of the device is capable of translation which varies from 90 degrees to about 0 degrees relative to the approximate plane of defect, bone segment, osteotomy or surface of another device. In some embodiments, rail R is used to establish and hold the spatial, i.e., translational, relationship between base B, such that each base B is capable of being translated along at least some of the length of rail R. In some embodiments, each retaining element RE has a first end operatively coupled to a base B and a second end constructed and arranged to hold at least one of the one or more bone segments or bone fragments, e.g., via tines T.

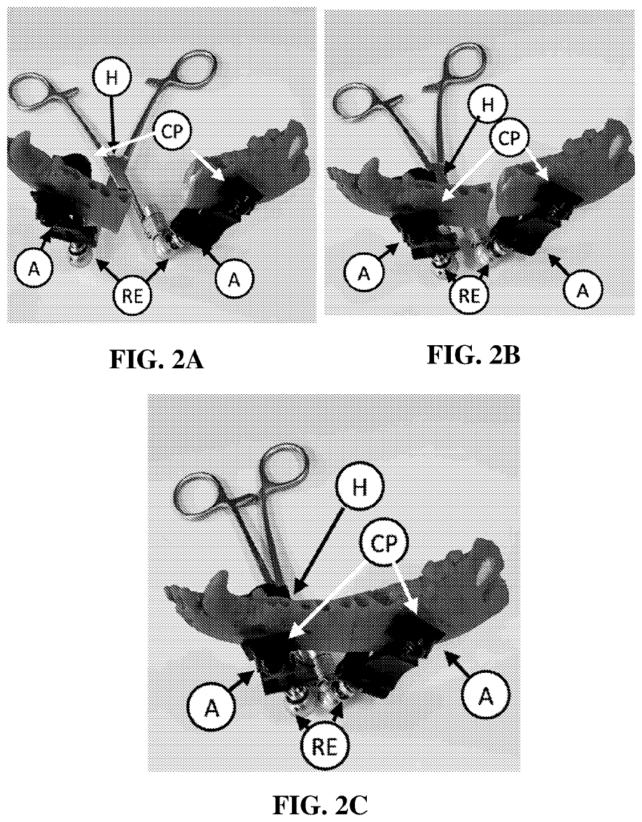

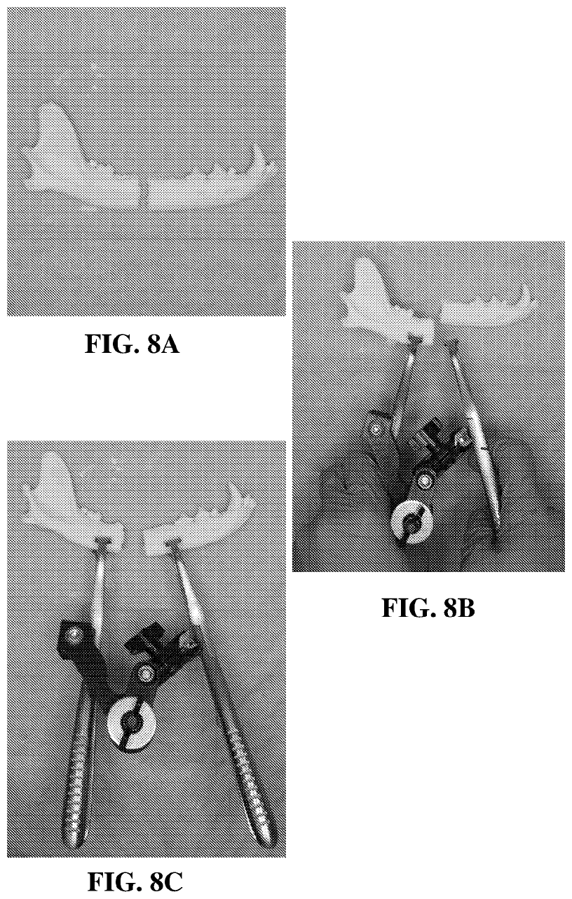

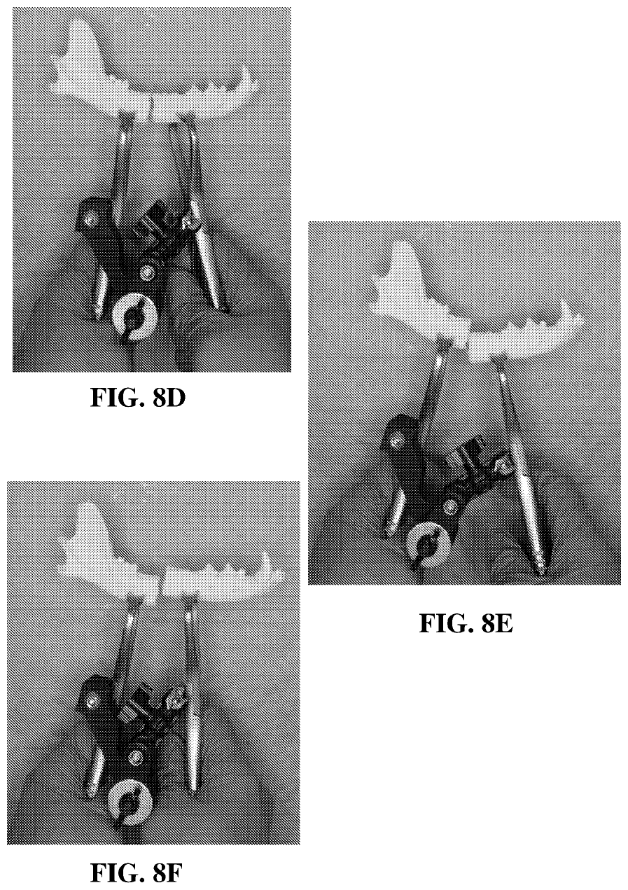

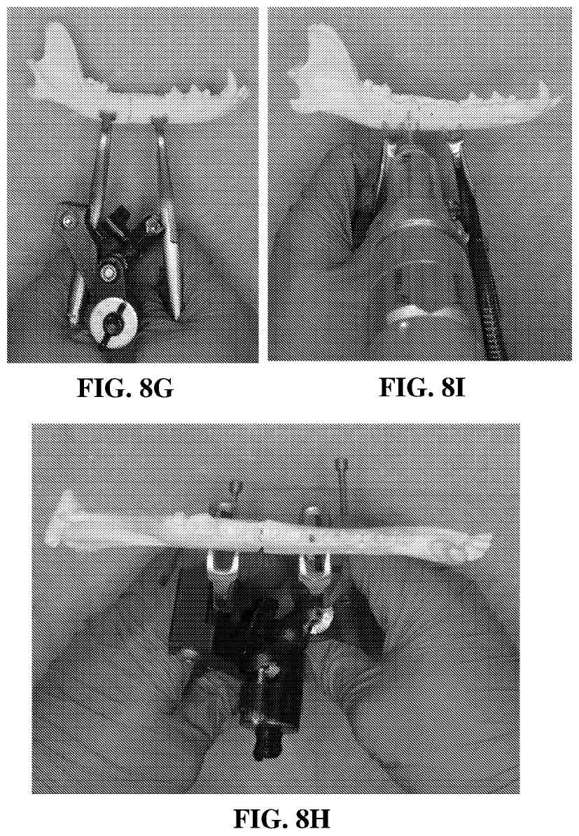

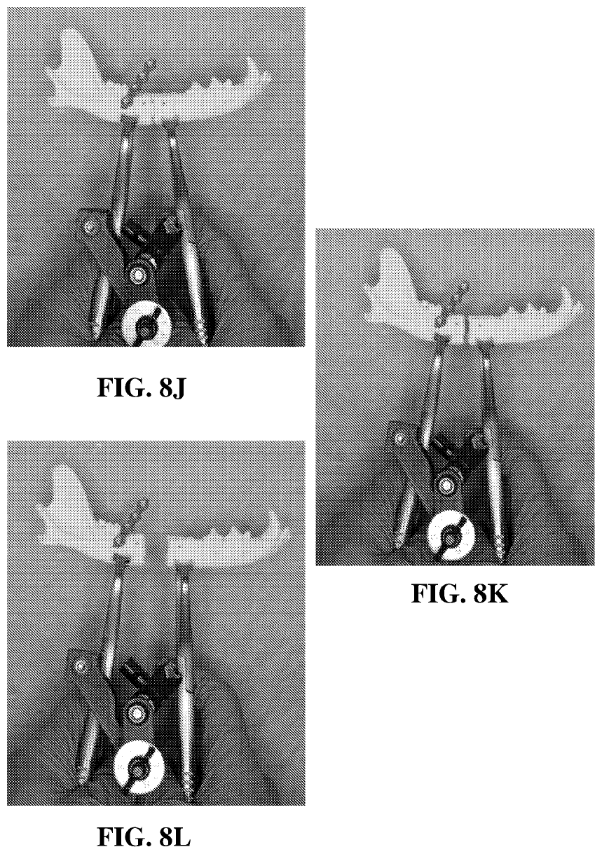

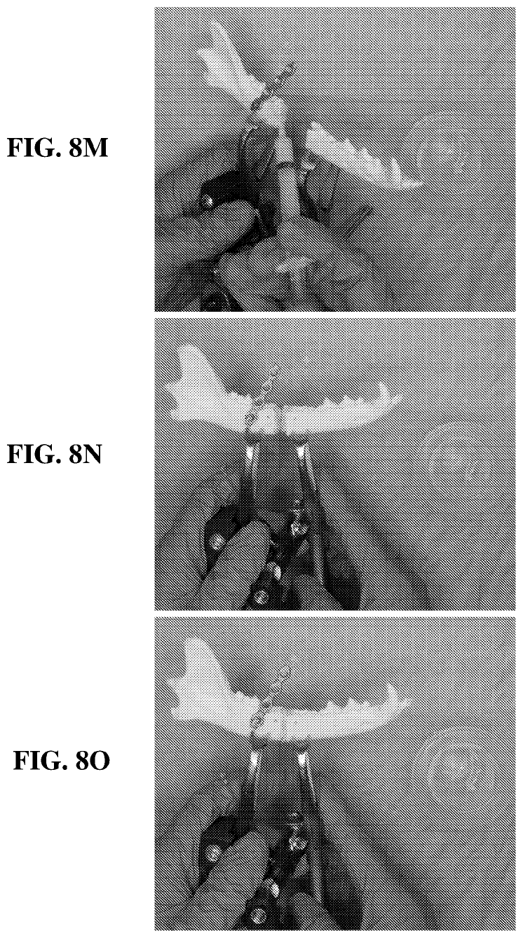

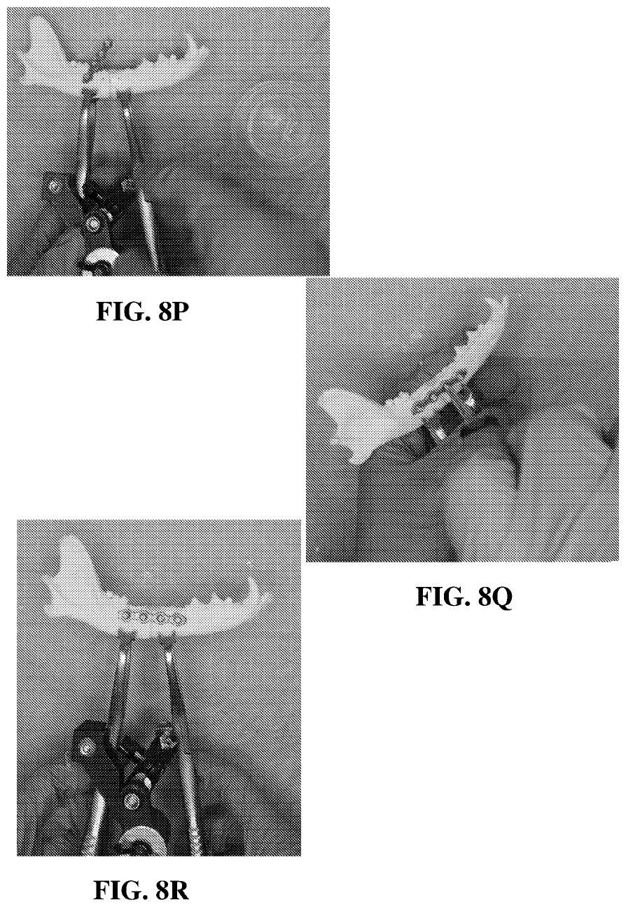

In another aspect, the present disclosure features a hinged fixation device, HFD, as illustrated in FIGS. 2A-2C and 8A-8R. In some embodiments, the relative angular displacement of the hinged fixation device HFD about the hinge is between about 2 degrees and about 120 degrees. An exemplary use of an HFD may be for the fixation of small bones and their fragments. In some embodiments, said HFD comprises a hinged component H, e.g., a base comprising one or more arms A connected by a hinge, and at least two retaining elements RE. In some embodiments, the HFD comprises additional components, including additional retaining elements RE, connectors C, bracing elements BE, stops S, tines T, or other such similar features and combinations thereof. In some embodiments, each retaining element RE further comprises a base element B. In some embodiments, each of the at least two retaining elements REs has a first end slidably coupled along a length of one of the one or more arms A of the hinge H and a second end constructed and arranged to hold at least one of the one or more structures, e.g., bone segments, bone fragments, implantable devices, preformed regenerative material blocks, etc., via sharp tines T or other contacting surfaces.

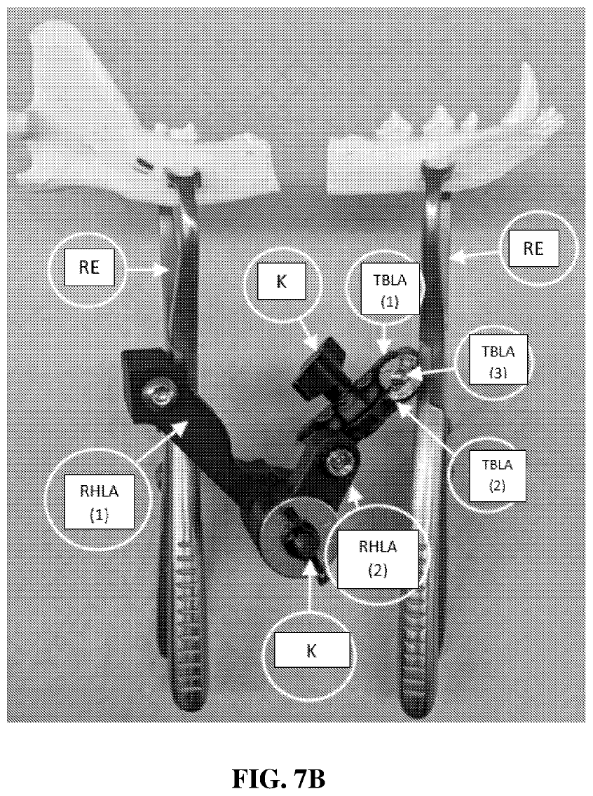

The present disclosure further discloses a fixation device comprising two or more linkage arms LA, as can be seen in FIGS. 7A-7B. In some embodiments, one of the linkage arms further comprises a rotary hinge-type linkage arm RHLA which allows at most, 1 degree of freedom of movement of the bone segments or structures attached thereto. In some embodiments, another of the linkage arms comprises a twin ball-joint type linkage arm TBLA which allows up to 3 degrees of freedom of movement of the bone segments or structures attached thereto. In some embodiments, RHLA further comprises two arms (RHLA(1) and RHLA(2)). In some embodiments, TBLA further comprises three arms (TBLA(1), TBLA(2), and TBLA(3)). In some embodiments, each linkage arm LA of the device further comprises a mechanical feature for locking motion of the device or the structures attached thereto. In some embodiments, this mechanical locking feature comprises a knob that can be reversibly actuated.

In some embodiments, both the hinged fixation device HFD and the rail fixation device RFD have varying iterations of the same elements, e.g., retaining elements RE, base element B, stops S, connectors C, tines T, arms A, hinges H, contact points CP.

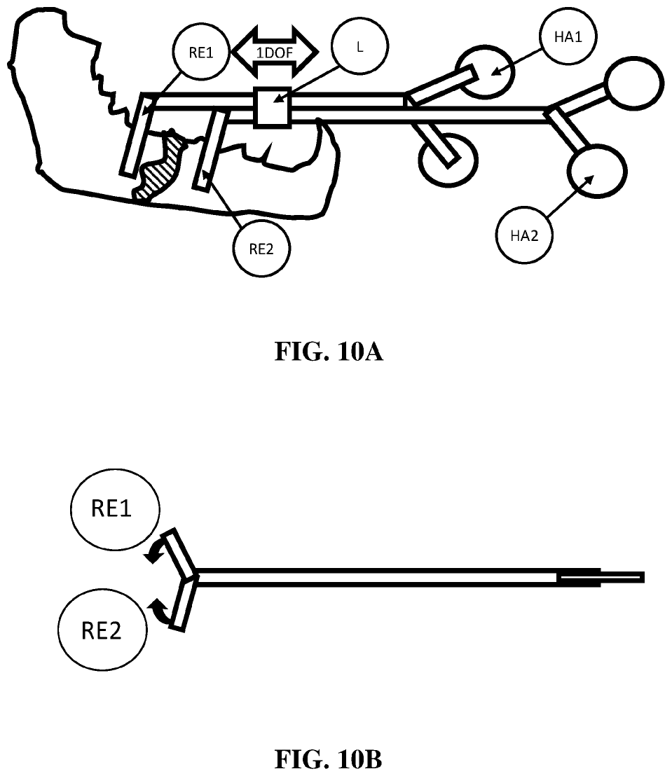

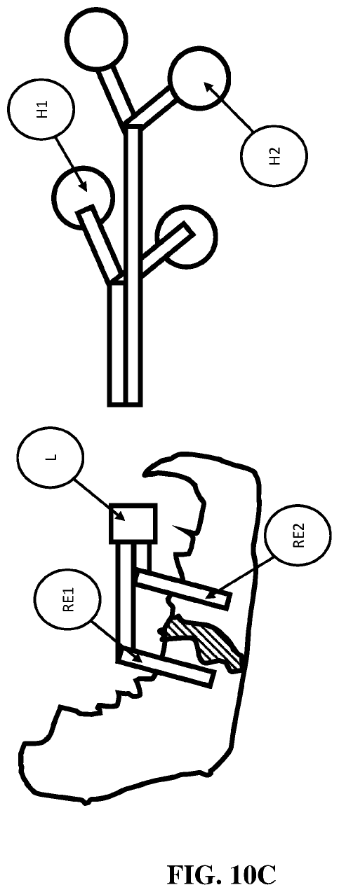

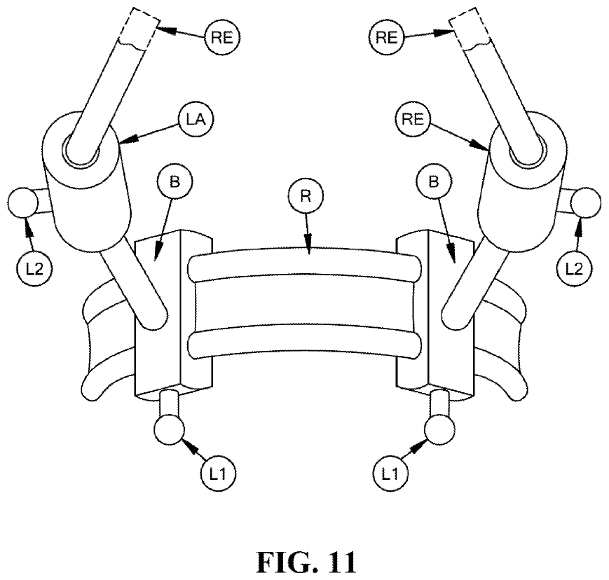

In some embodiments, the retaining element RE is elongated or extendable, as in FIGS. 10A-10C, to provide the user unhindered access to the surgical site. In some embodiments, the elongated retaining elements RE terminate in a set of handles for the user, as can be seen in FIG. 10C. In some embodiments the handles HA1, HA2 are detachable from the retaining elements RE to allow better access to the surgical site and to reduce the bulk of the device. In some embodiments, after detaching the handles HA1, HA2, the remainder of the device is left at the site post-operatively, e.g., the remainder of the device includes of a biocompatible resorbable material.

In some embodiments, retaining elements RE are fitted with mechanical stops S for limiting the translation of base elements B along the rail R. In some embodiments, stops S are capable of being selectively, repeatably, and reversibly affixed to the rail R. In some embodiments, stops S are positioned along a continuous linear scale along rail R. In some embodiments, stop S comprises a mechanical feature, e.g., a screw lock, which immobilizes the device and the structure with which the device is engaged. In some embodiments, the mechanical feature of stops S can be repeatably and reversibly locked in their position along rail R. In some embodiments, stops S can be irreversibly, i.e., permanently, locked in position along the rail. In some embodiments, the contact points CP, e.g., tines or other similar structural elements, of each retaining element RE are mechanically attached to base element B via a connector C.

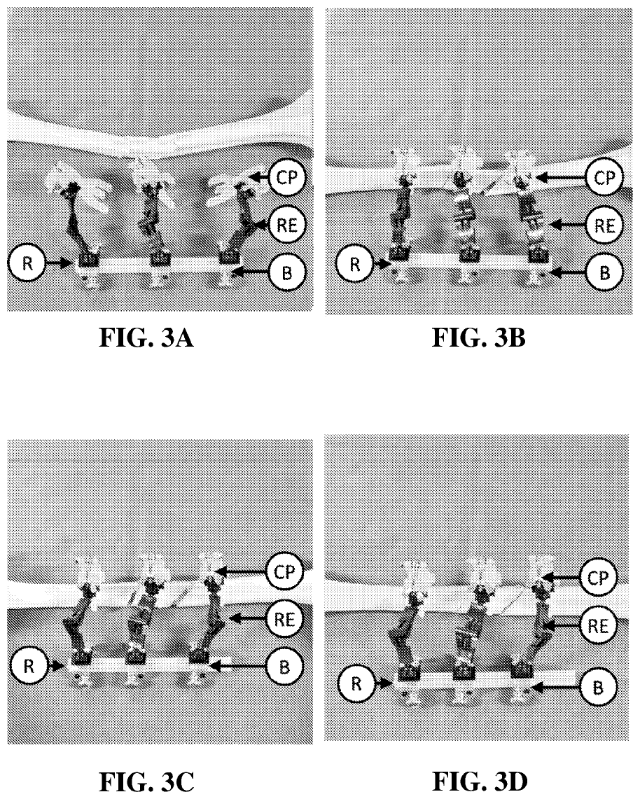

In some embodiments, contact points CP which engage the retained structure are located on retaining element RE arms A, as can be seen in FIGS. 1A-1B. In some embodiments, REs comprise two, three or more mutually opposable contact points CP. In some embodiments, contact points CP apply compressive force to immobilize the retained structure, as can be seen in FIGS. 3A-3D. In some embodiments the magnitude of the compressive force is between about 0.1 N and about 100 N.

In some embodiments, components of the connector, C, are movable relative one another. In some embodiments, relative movement of connector C allows adjustment of the spatial relationship of each contact point CP with the base element B and the structure being engaged. In some embodiments, the spatial relationship is adjusted via hinge motion, telescopic motion, ball-and-socket joints, or other similar geometries or combinations thereof. In some embodiments, connector C includes one or more, e.g., one, two, three, four or more, components which adjust the spatial relationship of RE and B. In some embodiments, the range of motion of each component relative one another provides up to three degrees of rotational freedom and up to three degrees of translational freedom such that the spatial configuration of each retained bone segment or structure can be adjusted to a preferred alignment. In some embodiments, connector C is reusable. In some embodiments, C is single use and disposable. In some embodiments, connector C is rigid. In some embodiments, the rigidity of connector C is adjustable or reversible. In some embodiments, the rigidity of C is adjusted via use of a cam mechanism or compression screw mechanism. In some embodiments, irreversible rigidity of connector C is accomplished via the use of a cement, e.g., cyanoacrylate, at contact zones between components of C. In some embodiments, irreversible rigidity is accomplished through the use of crimping, swaging, or other plastic deformation method or technique.

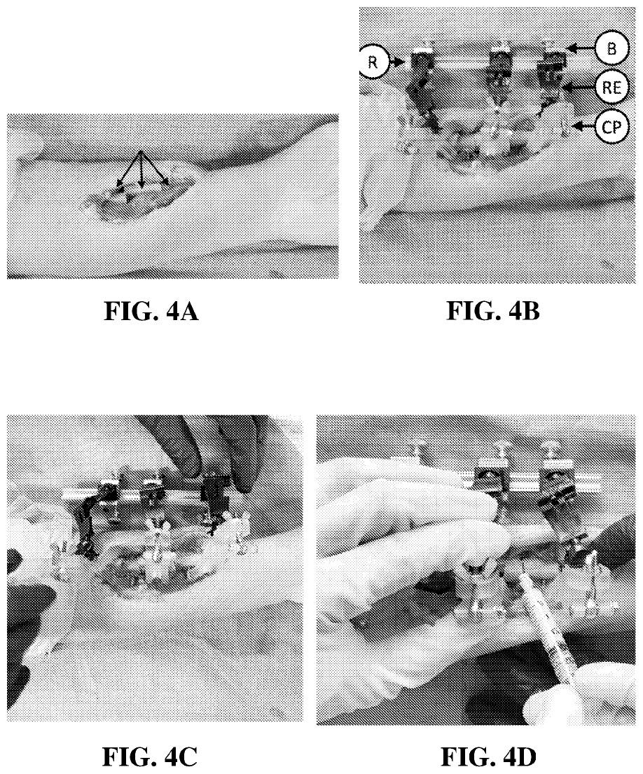

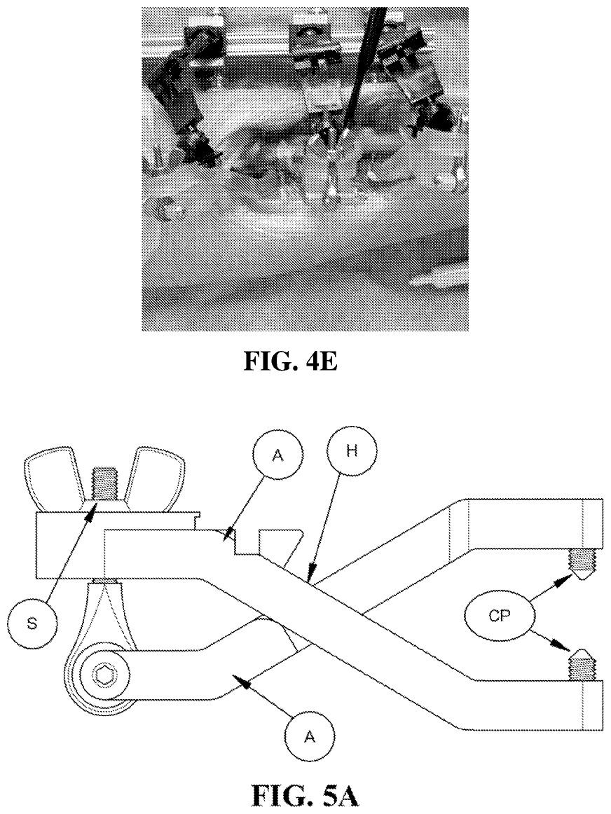

In some embodiments, retaining elements RE possess only one degree of freedom along the rotational axis, e.g., in an HFD. In some embodiments, retaining elements RE possess only one degree of freedom along the translational axis, e.g., when attached to the rail as part of an RFD. In some embodiments, there are more than two, e.g., three, four or more, retaining elements RE. In some embodiments, each retaining element RE engages a separate structure, e.g., bone fragment, or one part of the same structure, e.g., bone fragment or entire bone. In some embodiments, there are more than one, e.g., two, three or more, retaining elements RE engaging any one structure, e.g., bone segment or bone fragment, in order to improve the structural stiffness of the device construction or to immobilize the structures more definitively. In some embodiments, there are one, two, three or more retaining elements RE attached to each base B. FIGS. 4A-4E depict an exemplary use of three rail fixation devices RFD for fixating a fractured cadaver long bone. FIG. 4A depicts fractured bone segments which are displaced out of their proper anatomical alignment. FIG. 4B depicts three RFDs which are attached to a rail element RE via base element B at one end and grasping a portion of the bone segments to be fixated via arms A and contact points CP. FIG. 4C depicts the rail fixation devices RFD positioning each bone segment in a preferred alignment. FIG. 4D depicts the bone segments being separated while maintaining their spatial relationship in order to create a gap between the bone segments. FIG. 4D depicts an adhesive composition being delivered to the gap between the bone segments for definitive fixation. FIG. 4E depicts the RFDs holding each bone segment in its preferred alignment and applying a compressive force while the adhesive composition cures.

In some embodiments, the gap that is created by the device between the bone segments that are positioned is filled with a solid object or implant that is fixated to the bone with an adhesive composition. In some embodiments, the solid object or implant may be a metallic, e.g., titanium, implant or be a bone substitute comprising a solidified form of the adhesive composition. In further embodiments, the shape and size of the solid object or implant can be sized to fit or match the profile or outer contour of the gap. In some embodiments the solid object or implant is able to withstand stress, e.g., tensile, shear, bending, or torsion, of least 100 kPa.

In some embodiments, retaining elements RE include a standardized interface, e.g., threaded element, friction retained element, anti-rotational channel element, or other similar element, to accommodate attachment of a standardized or customizable structure, e.g., a preformed shape comprising a synthetic structure, e.g., a regenerative biomaterial block formed in a shape complementary to the form and size of the one or more RE.

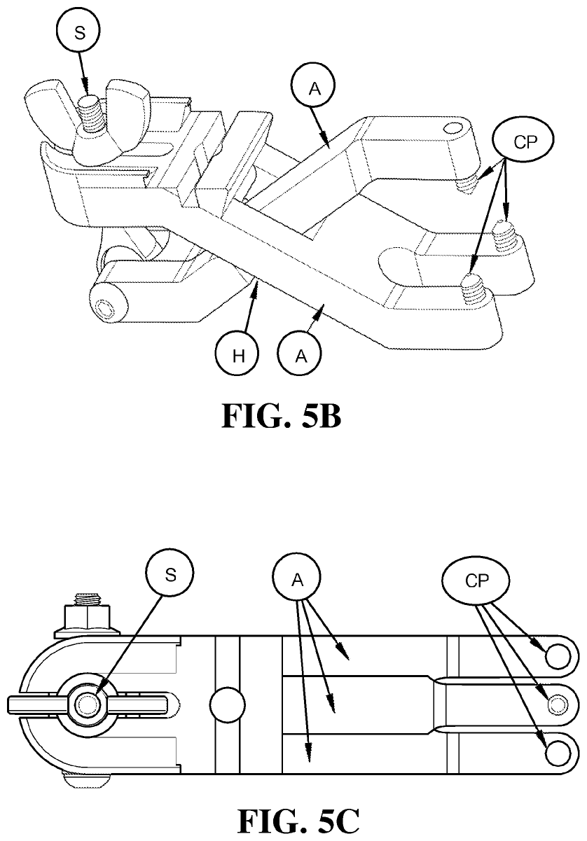

In some embodiments, retaining elements RE include movable arms A. In some embodiments, said arms A move relative one another in a rotational motion, e.g., in a hinged fixation device HFD, or in a translational motion, e.g., in a rail fixation device RFD. In some embodiments, a retaining element RE is comprised of a contacting surface, e.g., a flat surface, convex surface or concave, e.g., cylindrical surface, conical surface, capable of supporting a structure without grasping via contact points CP or tines T. In some embodiments, contact points CP are located along the retaining element arms A of the device. In some embodiments, said CPs comprise a number of tines T which are used to grasp the bone segments or structures to be fixated. FIGS. 5A-5C depict exemplary such retaining elements RE, and their components: stops S, arms A, contact points CP, and hinge H.

In some embodiments, retaining elements RE comprise stiff, slender portions, e.g., tines T, which follow the path made by contact points CP as they are moved towards the bone segments or structures to be grasped or retained. In some embodiments, contact points CP are at the termination of tines T. In a hinged fixation device HFD, said tines T may be of an arcuate design. In a rail fixation device RFD, said tines T may be in a rectilinear design. In some embodiments, said contact points CP and tines T are one and the same. In some embodiments, tines T are directed at right angles to RE arms A. In some embodiments, said tines lie parallel to the path of movement of contact points CPs as T approaches the surface of the structure to be engaged, e.g., bone segment. In some embodiments, tines T are at an angle which can be varied by an end user and reversibly locked into a position. In some embodiments, tines T are integrated into a retaining element RE. In some embodiments, tines T are removable or detachable from the device. In some embodiments, the tines T on a retaining element RE can be of varying length. In some embodiments, tines T extend from arms A. In some embodiments, the slender structure of T permits penetration into and through soft tissue with minimal damage. In some embodiments the dimensions, e.g., length and thickness, and physical characteristics, e.g., stiffness, of tines T may vary based on the anticipated clinical application. For example, application of the device to the bones of the hand or foot may not require tines T as long, stiff, or strong as that involving larger, thicker bones. In some embodiments, tines T include substantially parallel walls. In some embodiments, tines T include a segment that is substantially parallel but terminates with a tapered, e.g., conical, segment.

In some embodiments, tines T are rotatable relative to arms A, or translatable and rotatable. In some embodiments, rotation of T is elective around the long axis of T in the region of contact with A. In some embodiments, rotation is elective around an angle to the long axis of T, e.g., perpendicular to the long axis of T at the region of contact with arms A. In some embodiments, T is movable relative A, e.g., translationally retractable or extensible without freedom to rotate about the long axis. In some embodiments, T is selectively reversibly or irreversibly fixable to A. In some embodiments, the extension and retraction of T is governed by threads within the retaining arm A channel and on the surface of T which contacts retaining arm channel. In some embodiments, tines T are movable relative arm A through a channel of the arm A. In some embodiments, said channel accommodates passage of a drill bit, e.g., through a drill sleeve. In some embodiments, tines T engage the bone at the same location where the drill bit engages the bone. In some embodiments, tines T engage the bone with a thread. In some embodiments, tines T comprise a channel along a part or totality of its length, which terminates near contact point CP of the retaining element RE, e.g., in the form of a cannula or trocar. In some embodiments, tines T are rotatable relative contact points CP with such a structural feature to allow a seal to form, wherein such seal allows development of hydrostatic pressure sufficient to conduct a material through the channel. In some embodiments, said channel is used to conduct materials through it such as a liquid, a gas, a suspension, a gel, or some other similar such material. In some embodiments, said material being conducted through the channel is an adhesive composition, e.g., the adhesive composition as described herein.

In some embodiments, the devices described herein further comprise bracing elements BE which stabilize the rail component R against a stable surface, e.g., operating room tabletop, a permanent stand, or another fixed structural point. In some embodiments, wherein the device is a rail fixation device RFD, bracing element BE stabilizes the rail R. In some embodiments, wherein the device is a hinged fixation device HFD, the bracing element BE stabilizes the base B against a stable surface. In some embodiments, a bracing element BE attaches to the rail R or base element B via an interface element. Said bracing element BE may include one, two, three or more rigid components. In some embodiments, the bracing element BE supports the weight of the device to reduce force on the structures being fixated. One or more bracing elements BE may be used simultaneously during a procedure. In some embodiments, a segment of the bracing element BE is adjustable in length through the use of telescoping features between components of the brace. In some embodiments, the length of the bracing element BE is variable and electively and reversibly locked, e.g., by the use of a cam, compression screw, a jamming wedge, or other similar mechanical connection. In some embodiments, the length of the bracing element BE is selectively and irreversibly locked, e.g., by use of a cement, a crimp or another mechanical feature. In some embodiments, the bracing element BE is adjustable with respect to its form via the use of articulations, e.g., ball-and-socket joints or hinge joints. In some embodiments, said articulations are electively and reversibly locked in desired shape via a mechanical feature, e.g., the use of screws, by compressing the hinge knuckles together or limiting the range of motion with a stop S.

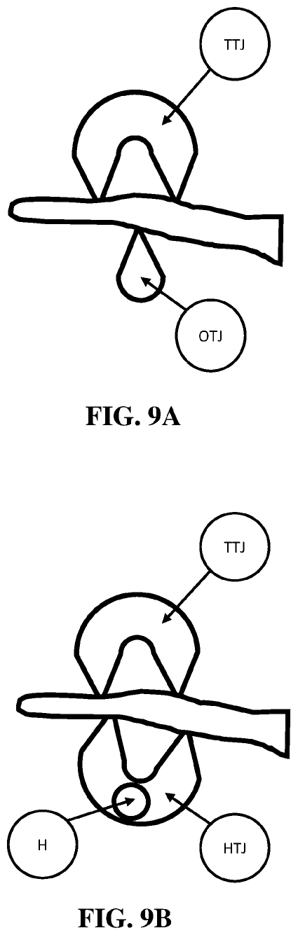

In some embodiments, the retaining element RE of a device includes a three-tined jaw which is suitable for use in limited access spaces, e.g., spaces which are less than 20 mm in dimension, as depicted in FIG. 9A. In some embodiments, retaining element RE includes a two-tined jaw TTJ at one end of the retaining element RE, and a one-tined jaw OTJ opposing the TTJ. In some embodiments, retaining element RE includes a four-tined jaw suitable for use with small or weak bone segments, as depicted in FIG. 9B. In some embodiments, such a retaining element RE includes one two-tined jaw TTJ, and an opposing hinged two-tined jaw HTJ. In some embodiments, each jaw grasps the bone segment via the tines T. The retaining element RE components, e.g., tines T, and contacting points CP, and contacting surfaces, may be capable of sufficient elastic deformation to make engaging contact with irregularities of the engaged structure by accommodating these irregularities through elastic deformation of said components.

In some embodiments, the devices described herein may be used in conjunction with other fixation devices, e.g., k-wires, to stabilize the structures. In some embodiments, use of a k-wire provides stabilization for a structure to be fixated at a site more precarious than a long or flat bone, e.g., a dental procedure. For example, in dentistry, a k-wire may be wrapped around a structure to be fixated and abutting teeth to stabilize the structure, e.g., implant or broken tooth, to be fixated more securely while the adhesive composition cures.

The devices disclosed herein are capable of undergoing one or more sterilization processes before its use, e.g., dry or wet autoclave, gamma irradiation, electron beam irradiation, or ethylene oxide treatment, to prevent or mitigate bacterial contamination to the wound site. Sterilization may not compromise the materials of the device, determined by mechanical testing of the device. Said devices are capable of undergoing gamma irradiation up to 50 kGy, preferably between 10 and 40 kGy, or between 20 and 30 kGy.

The above devices and embodiments may be used separately or in conjunction with each other. Features of one device may be seen in use or as part of any other device.

### Adhesive Compositions

The present disclosure also features an adhesive composition for use with the device embodiments described herein. In some embodiments, the adhesive composition comprises a material that fills into or around the kerf or space between the bone segments. In some embodiments, an adhesive composition is utilized to partially or fully seal the kerf line in the osteotomy. A partial fill refers to enabling permanent fixating of the bone segments, whereas a full fill refers to sealing the kerf space with a depth equaling the thickness of the flap or the length equaling the run of the osteotomy. In some embodiments, said adhesive composition comprises a resorbable material, e.g., a material which is resorbed by a subject and may be replaced with host tissue. In some embodiments, the adhesive composition comprises a mineral-based compound comprising a multivalent metal, an organic compound and an aqueous medium. In some embodiments, said multivalent metal may be a calcium salt, e.g., calcium phosphate. In some embodiments, the multivalent metal comprises tricalcium phosphate (e.g., α-tricalcium phosphate or β-tricalcium phosphate) or tetracalcium phosphate. In some embodiments, the multivalent metal comprises tricalcium phosphate. In some embodiments, the multivalent metal comprises tetracalcium phosphate. In some embodiments, said organic compound comprises an organophosphate or organic acid.

In some embodiments, the adhesive composition comprises an organic compound of Formula (I):

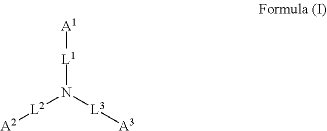

wherein each of A¹, A², and A³ is independently selected from an acidic group (e.g., a carboxyl or phosphonyl), and each of L¹, L², and L³ is independently a bond, alkylene (e.g., C₁-C₆ alkylene), or heteroalkylene (e.g., C₁-C₆ heteroalkylene).

In some embodiments, each of A¹, A², and A³ is independently a carboxyl or phosphonyl.

In some embodiments, A¹ is carboxyl, and A² and A³ are phosphonyl. In some embodiments, A¹, A² and A³ are phosphonyl.

In some embodiments, each of L¹, L², and L³ is C₁-C₃ alkylene. In some embodiments, each of L¹, L², and L³ is C₁ alkylene.

In some embodiments, the compound of Formula (I) is a compound of Formula (I-a) or (I-b):

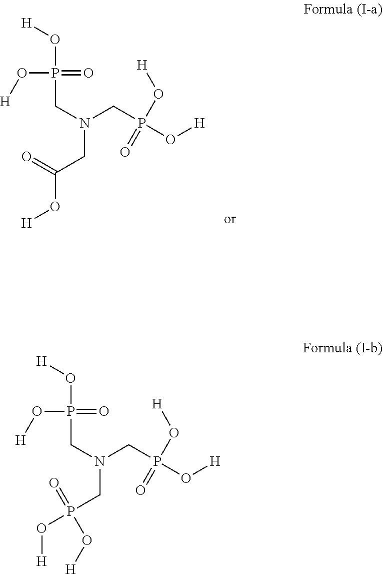 [SMILES: O=C(O)CN(CP(=O)(O)O)CP(=O)(O)O ; O=P(O)(O)CN(CP(=O)(O)O)CP(=O)(O)O]

In some embodiments, the adhesive composition comprises an organic compound of Formula (II):

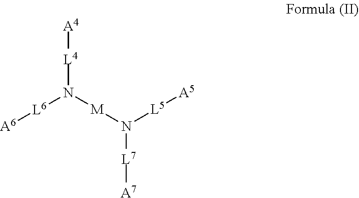

wherein each of A⁴, A⁵, and A⁶, is independently selected from an acidic group (e.g., a carboxyl or phosphonyl), A⁷ is selected from an acidic group (e.g., a carboxyl or phosphonyl), a hydrogen atom, an alkyl, an aryl, a hydroxy group, a thio group, and an amino group, each of L⁴, L⁵, L⁶, and L⁷ is independently bond, alkylene (e.g., C₁-C₆ alkylene), or heteroalkylene (e.g., C₁-C₆ heteroalkylene), and M is alkylene (e.g., C₁-C₆ alkylene) or heteroalkylene (e.g., C₁-C₆ heteroalkylene).

In some embodiments, A⁴, A⁵, A⁶ and A⁷ are carboxyl.

In some embodiments, L⁴, L⁵, L⁶, and L⁷ are C₁-C₃ alkylene. In some embodiments, L⁴, L⁵, L⁶, and L⁷ are C₁ alkylene.

In some embodiments, M is C₁-C₄ alkylene. In some embodiments, M is C₂ alkylene. In some embodiments, M is C₃ alkylene. In some embodiments, M is C₁-C₆ heteroalkylene. In some embodiments, M is C₆ heteroalkylene. In some embodiments, M is bis(ethyleneoxy)ethylene. In some embodiments, M includes side chains. In some embodiments, M includes multiple side chains. In some embodiments, M includes one or multiple carboxymethylene side chains. In some embodiments, M includes one or multiple N-carboxymethylene groups or N-hydroxymethylene groups.

In some embodiments, the compound of Formula (II) includes three, four, five, six, or more N-carboxymethylene groups.

In some embodiments, the compound of Formula (II) comprises ethylenediamine tetraacetic acid (EDTA).

In some embodiments, the compound of Formula (II) is a compound of Formula (II-a), (II-c), (II-d), (II-e), or (II-f):

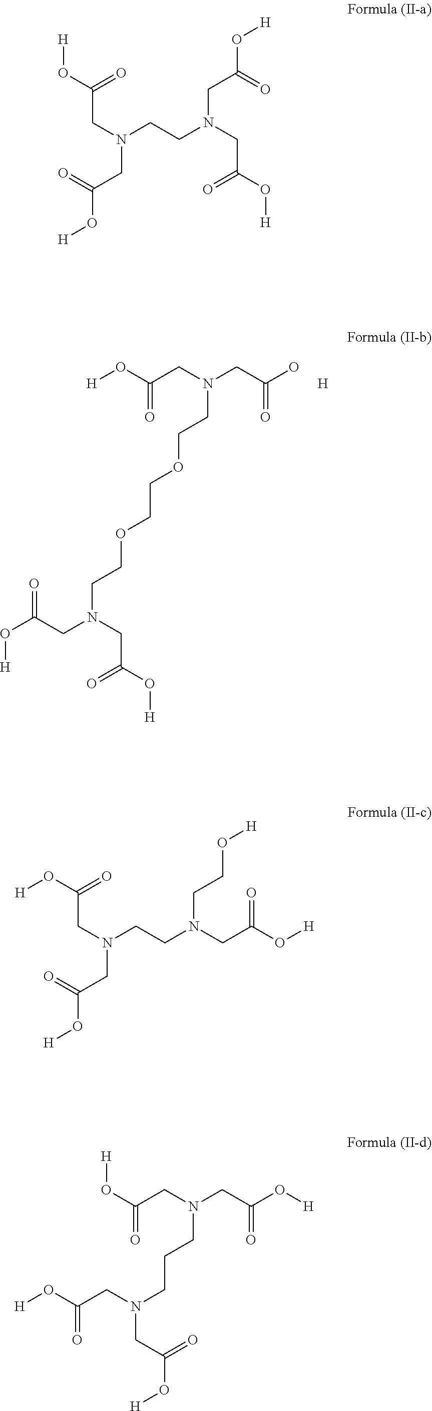

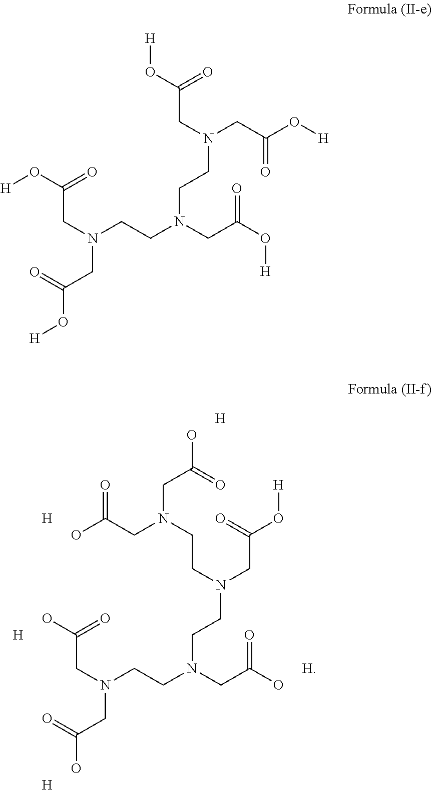

In some embodiments, the adhesive composition comprises an organic compound of Formula (III):

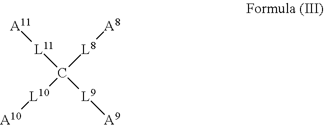

wherein each of A⁸ and A⁹ is independently selected from an acidic group (e.g., a carboxyl or phosphonyl), each of A¹⁰ and A¹¹ is independently selected from an acidic group (e.g., a carboxyl or phosphonyl), a hydrogen atom, an alkyl, aryl, a hydroxy group, a thio group, and an amino group, each of L⁸, L⁹, L¹⁰ and L¹¹ is independently bond, alkylene (e.g., C₁-C₆ alkylene), or heteroalkylene (e.g., C₁-C₆ heteroalkylene).

In some embodiments, A⁸, A⁹, and A¹⁰ are carboxyl. In some embodiments, A¹⁰, A¹¹, are a hydrogen atom. In some embodiments, A¹¹ is a hydroxy or amino group. In some embodiments, L⁸, L⁹, L¹⁰, and L¹¹ are a bond. In some embodiments, L⁸ and L⁹ are C₁-C₃ alkylene. In some embodiments L¹¹ is a heteroalkylene (e.g., C₁-C₆ heteroalkylene). In some embodiments L¹¹ is methylenethiomethylene.

In some embodiments, the compound of Formula (III) comprises citric acid or malonic acid.

In some embodiments, the compound of Formula (III) is a compound of Formula (III-a), (II-c), or (III-d):

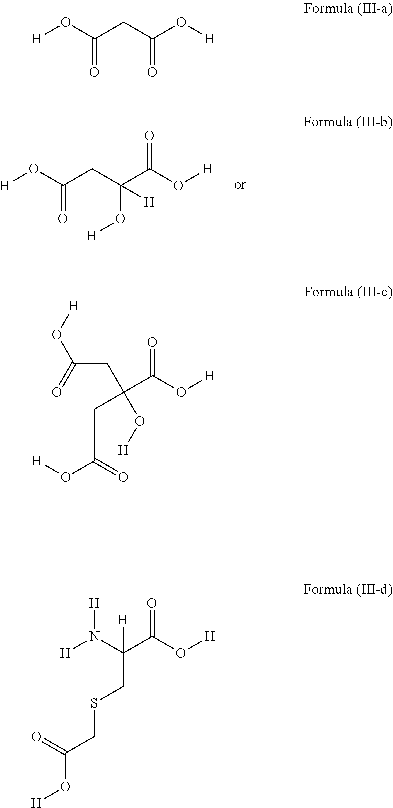 [SMILES: O=C(O)CC(=O)O ; O=C(O)CC(O)C(=O)O ; O=C(O)CC(O)(CC(=O)O)C(=O)O ; NC(CSCC(=O)O)C(=O)O]

In some embodiments, the adhesive composition comprises an organic compound of Formula (IV):

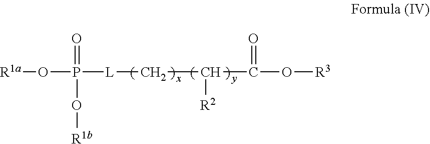

wherein L is O, S, NH, or CH₂, each of R^{1a} and R^{1b} is independently H, an optionally substituted alkyl, or an optionally substituted aryl, R² is H, NR^{4a}R^{4b}, C(O)R⁵, or C(O)OR⁵, R³ is H, an optionally substituted alkyl, or an optionally substituted aryl, each of R^{4a} and R^{4b} is independently H, C(O)R⁶, or an optionally substituted alkyl, R⁵ is H, an optionally substituted alkyl, or an optionally substituted aryl, R⁶ is an optionally substituted alkyl or an optionally substituted aryl, and each of x and y is independently 0, 1, 2, or 3.

In some embodiments, L is O or S. In some embodiments, L is O. In some embodiments, each of R^{1a} and R^{1b} is independently H. In some embodiments, L is O, and each of R^{1a} and R^{1b} is H.

In some embodiments, R² is selected from H, NR^{4a}R^{4b}, and C(O)R⁵. In some embodiments, R² is NR^{4a}R^{4b}. In some embodiments, R² is NR^{4a}R^{4b} and each of R^{4a} and R^{4b} is independently H.

In some embodiments, L is O, each of R^{1a} and R^{1b} is independently H, R² is NR^{4a}R^{4b}, and each of R^{4a} and R^{4b} is independently H.

In some embodiments, R³ is H. In some embodiments, L is O, each of R^{1a} and R^{1b} is independently H, R² is NR^{4a}R^{4b}, each of R^{4a} and R^{4b} is independently H, and R³ is H.

In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1.

In some embodiments, the compound of Formula (IV) is phosphoserine.

In some embodiments, the adhesive composition comprises an organic compound of Formula (V):

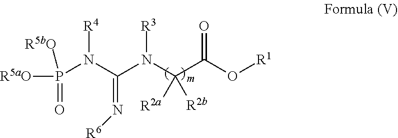

wherein R¹ is H, optionally substituted alkyl, optionally substituted alkyl, optionally substituted alkyl, optionally substituted aryl, or optionally substituted heteroaryl, each of R^{2a} and R^{2b} is independently H, optionally substituted alkyl, hydroxy, alkoxy, or halo, each of R³ and R⁴ is independently H or optionally substituted alkyl, each of R^{5a} and R^{5b} is independently H, optionally substituted alkyl, optionally substituted alkyl, optionally substituted alkyl, optionally substituted aryl, or optionally substituted heteroaryl, R⁶ is H or optionally substituted alkyl, and m is 1, 2, 3, 4, or 5.

In some embodiments, R¹ is H. In some embodiments, each of R^{2a} and R^{2b} is independently H. In some embodiments, m is 1. In some embodiments, each of R³ and R⁴ is H.

In some embodiments, each of R^{5a} and R^{5b} is independently H. In some embodiments, R⁶ is H.

In some embodiments, the compound of Formula (V) is a phosphocreatine. In some embodiments, the compound of Formula (V) is Formula (V-a):

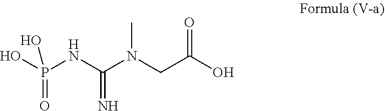

In some embodiments, the compound of Formula (V) is phosphocreatine (e.g., Formula V-a).

In some embodiments, the adhesive composition comprises an organic compound of Formula (VI):

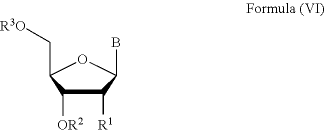

wherein B is a nucleobase, R¹ is H, OR⁴, or halo, R² is H, optionally substituted alkyl, optionally substituted alkenyl, optionally substituted aryl, optionally substituted heteroaryl, optionally substituted cycloalkyl, or optionally substituted heterocyclyl, R³ is H, optionally substituted alkyl, or a phosphate moiety (e.g., monophosphate or diphosphate), and R⁴ is H, optionally substituted alkyl, optionally substituted alkenyl, optionally substituted aryl, optionally substituted heteroaryl, optionally substituted cycloalkyl, or optionally substituted heterocyclyl.

In some embodiments, B is a naturally occurring nucleobase or a non-naturally occurring nucleobase. In some embodiments, B comprises adenine, cytosine, guanosine, thymine, or uracil. In some embodiments, each of R¹, R², and R³ is H. In some embodiments, R³ is a phosphate group, e.g., a monophosphate, diphosphate, or triphosphate.

In some embodiments, the compound of Formula (VI) is Formula (VI-a) or (VI-b):

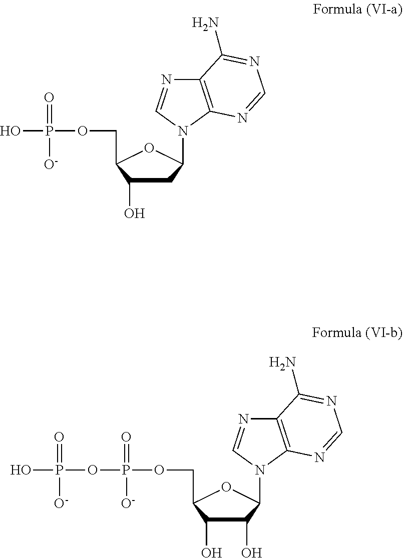

For example, the compound of Formula (VI) is 2′-deoxyadenosine monophosphate or 2′-deoxyadenosine diphosphate.

In some embodiments, an adhesive composition further comprises an additive. An additive may be used to impart additional functionality to the composition of the disclosure, such as improving or affecting the handling, texture, durability, strength, or resorption rate of the material, or to provide additional cosmetic or medical properties. The additive may comprise a salt (e.g., calcium carbonate, calcium bicarbonate, sodium carbonate, sodium bicarbonate, sodium chloride, or potassium chloride). The additive may comprise one or more of a filler, a formulation base, an abrasive (e.g., bone fragment), a coloring agent (e.g., dye, pigment, or opacifier), a flavoring agent (e.g., sweetener), or a polymer. The additive may comprise a viscosity modifier (e.g., a polyol (e.g., glycerol, mannitol, sorbitol, trehalose, lactose, glucose, fructose, or sucrose)). The additive may comprise a medication that acts locally (e.g., an anesthetic, coagulant, clotting factor, chemotactic agent, and agent inducing phenotypic change in local cells or tissues), a medication that acts systemically (e.g., analgesic, anticoagulant, hormone, enzyme co-factor, vitamin, pain reliever, anti-inflammatory agent, chemotactic agent, or agent inducing phenotypic change in local cells or tissues), or an antimicrobial agent (e.g., antibacterial, antiviral, or antifungal agent).

In some embodiments, the additive comprises a polymer. The biologically active substances (e.g., medicines) in the categories above might include active substances or precursors, which become biologically active upon modification after interaction with the surrounding environment. The substances might be synthetic, semisynthetic, or biologically derived (e.g., peptides, proteins, or small molecules). The substances might include, but not be limited to anti-inflammatories (e.g., steroids, nonsteroidal anti-inflammatory drugs, cyclooxygenase inhibitors), complement proteins, bone morphogenic factors and proteins, hormones active locally or systemically (e.g., parathyroid hormone, calcitonin), or other small molecules (e.g., calciferols).

In some embodiments, the additive is a polymer. Suitable polymers incorporated as additives into the adhesive composition may contain functional groups that contains electronegative atoms as the bonding sites of the polymer surfaces to the available metal ions, such as electronegative carbonyl oxygen atom(s) of the ester group or electronegative nitrogen atom(s) of the amine group as the bonding sites of the polymer surfaces to the available metal ions. These functional groups can be either in the backbone chain of the polymer or in groups pendant to the polymer chain. These polymeric based compounds may include, but are not limited to, one or more of the following; poly(L-lactide), poly(D,L-lactide), polyglycolide, poly(O-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin and/or mixtures thereof. In addition, copolymers of the above homopolymers also can be used.

The general structural nature of a polymer (e.g., a polymer used as an additive in an adhesive composition described herein) may include a linear homo and copolymer, a cross linked polymer, a block polymer, a branched polymer, a hyper branched polymer, or a star shaped polymer. The polymers can be added to the formulation in the form of a solution, powder, fiber, resin, liquid crystal, hydrogel, chip, flake, granule, and the like. The polymeric material can be included directly within the adhesive composition or can be an adjunct that is applied in situ as the cement is applied to the bone.

In some embodiments, the composition comprises a plurality of said additives. In some embodiments, certain additives may be provided as powders or granules or solutes or any combination thereof. These powders may exhibit a mean particle size of about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The powder may have a mean particle size of less than about 1.000 mm. The particle size distribution may be multi-modal to include any combination of mean particle sizes as previously described. These granules may exhibit a mean granule size of about 0.050 mm to about 5 mm, about 0.100 to about 1.500 mm, about 0.125 to 1.000 mm, 0.125 to 0.500 mm, about 0.125 to 0.250 mm, about 0.250 to 0.750 mm, about 0.250 to 0.500 mm, about 0.500 to 1.00 mm, about 0.500 to 0.750 mm. The granule size distribution may be multi-modal to include any combination of mean granule sizes as previously described. The granules may be supplied with a various proportion of porosity and a various size of internal pores. The pores may communicate with granule surface. In some embodiments, the pores do not communicate with granule surface. In some embodiments, varying sizes of said powders or granules may be used in the adhesive composition.

In some embodiments, certain additives may be provided as fibers. In some embodiments, the fibers may exhibit a mean fiber diameter of about 0.010 mm to about 2 mm, about 0.010 mm to about 0.50 mm, or about 0.025 mm to about 0.075 mm. These fibers may exhibit a mean fiber length of about 0.025 mm to about 50.0 mm, about 0.50 mm to 10 mm, or about 1.00 mm to about 3.50 mm. The fiber diameter distribution or length distribution may be multi-modal to include any combination of mean fiber diameter or length.

In some embodiments, the multivalent metal salt comprises one or more alkaline earth metals, e.g., beryllium, magnesium, barium, radium, strontium, or calcium. In some embodiments, the multivalent metal salt may comprise a mixed salt of several metal ions, e.g., a mixed salt of alkali earth metal ions. In some embodiments, the multivalent metal salt comprises calcium. In some embodiments, the multivalent metal salt comprises calcium and phosphate. In some embodiments, the multivalent metal salt comprises tetra-calcium phosphate. In some embodiments, the composition comprises a plurality of multivalent metal salt compounds. In some embodiments, the plurality comprises tetra-calcium phosphate and at least one other multivalent metal salt compound. In some embodiments, the multivalent metal salt comprises hydroxyapatite. In some embodiments, the multivalent metal salts comprise tricalcium phosphate. In some embodiments, the tricalcium phosphate comprises either alpha tricalcium phosphate or beta tricalcium phosphate. In some embodiments, the multivalent metal salts comprise an oxide. In some embodiments, the multivalent metal salt is calcium oxide. In some embodiments, the multivalent metal salt compound does not comprise tetra-calcium phosphate. In some embodiments, the composition comprises tricalcium phosphate and calcium oxide.

In some embodiments, the multivalent metal salt is initially provided as a powder or as a granule. These powders may exhibit a mean particle size of about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The powder may have a mean particle size of less than about 1.000 mm. The particle size distribution may be multi-modal to include any combination of mean particle sizes as previously described. These granules may exhibit a mean granule size of about 0.050 mm to about 5 mm, about 0.100 to about 1.500 mm, about 0.125 to 1.000 mm, 0.125 to 0.500 mm, about 0.125 to 0.250 mm, about 0.250 to 0.750 mm, about 0.250 to 0.500 mm, about 0.500 to 1.00 mm, about 0.500 to 0.750 mm. The granule size distribution may be multi-modal to include any combination of mean granule sizes as previously described. The granules may be supplied with a various proportion of porosity and a various size of internal pores. The pores may communicate with each other. The pores may communicate with granule surface. In some embodiments, the pores do not communicate with each other. In some embodiments, the pores do not communicate with granule surface. In some embodiments, varying sizes of said powders or granules may be used in the adhesive composition.

In the present disclosure, the multivalent metal salts (e.g., tetra-calcium phosphate) may react with a compound of a Formula (e.g., Formula (I), Formula (II), Formula (III), Formula (IV), Formula (V), or Formula (VI), or a combination thereof) to form an adhesive composition when combined with an aqueous medium.

In some embodiments, the aqueous medium comprises water (e.g., sterile water), saliva, buffers (e.g., sodium phosphate, potassium phosphate, or saline (e.g., phosphate buffered saline)), blood, blood-based solutions (e.g., plasma, serum, bone marrow), spinal fluid, dental pulp, cell-based solutions (e.g., solutions comprising fibroblasts, osteoblasts, platelets, odontoblasts, stem cells (e.g., mesenchymal stem cells) histiocytes, macrophages, mast cells, or plasma cells), or combinations thereof in the form of aqueous solutions, suspensions, and colloids. In some embodiments, the aqueous medium comprises sterile water, distilled water, deionized water, sea water, or fresh water. In some embodiments, the aqueous medium comprises water from the environment, e.g., fresh water, salt water or brackish water from the oceans, seas, bays, rivers, streams, ponds or other moving or standing water sources.

In some embodiments, an adhesive composition is self-setting or light-cured upon activation. In some embodiments, an adhesive composition is mixed at the time of use with an activator, e.g., an aqueous medium, to initiate self-setting. In some embodiments, said aqueous medium comprises a blood-based product, water, or other aqueous medium, e.g., compatible with bodily fluids. In an embodiment, said aqueous medium comprises water. To form an exemplary bone substitute, for example, tetracalcium phosphate and phosphoserine are mixed with water. In some embodiments, the combined weight of the multivalent metal compound and the organic-based compound comprises between about 10% to 90% of the total composition, e.g., 10% to 75% or 25 to 50% w/w, w/v, or v/v of the total weight of all components in the adhesive composition. In an embodiment, the aqueous medium is present in an amount up to about 10% or more, e.g., 15%, 20%, 25%, 30%, or 35%, w/w, w/v, or v/v of the total weight of all components in the adhesive composition. For example, an adhesive composition may include the aqueous medium of about 35% w/w, w/v, or v/v of the total composition. In some embodiments, the adhesive composition further comprises an additive, such as a salt, a filler, a viscosity modifier, an antibiotic, or a medication.

The amount of compound of a Formula (i.e., Formula (I), Formula (II), Formula (III), or Formula (IV), or a combination thereof) may vary, e.g., between about 10% to about 90% weight by weight (w/w) of the total composition. In some embodiments, the amount of compound of a Formula (i.e., Formula (I), Formula (II), Formula (III), or Formula (IV), or a combination thereof) is in the range of about 10% to about 90%, about 15% to about 85%, about 20% to about 80%, about 30% to about 75%, about 40% to about 70%, or about 50% to about 65% w/w of the total composition. In other embodiments, the amount of compound of a Formula (i.e., Formula (I), Formula (II), Formula (III), or Formula (IV), or a combination thereof) is in the range of about 5% to about 95%, about 10% to about 85%, about 15% to about 75%, about 20% to about 65%, about 25% to about 55%, or about 35% to about 50% w/w of the total composition.

In some embodiments, the adhesive composition disclosed herein is bioresorbable, e.g., disperses and is replaced with native tissue over time. In some embodiments, the adhesive composition is formed by mixing an organic compound (e.g., a compound of Formulas (I), (II), (III), (IV), (V), or (VI)) and multivalent metal salt with an aqueous medium. In some embodiments, said adhesive composition may be applied in its fluid or semi-solid state by an injection delivery device or similar application means into or around the kerf or space between the bone segments. In some embodiments, the adhesive composition has a tacky state for about 30 seconds to about 20 minutes upon activation through mixing with the aqueous medium. For example, the adhesive composition, when mixed, has a working time of about 15 seconds to about 5 minutes after mixing. In some embodiments, the adhesive composition has a tacky state for approximately 12 minutes upon mixing.

The adhesive compositions described herein may have a tacky state after mixing with an aqueous medium. This tacky property is retained for a number of days (e.g., up to 7 days, up to 3 days, up to 1 day), up to hours (e.g., up to 12 hours, up to 4 hours, up to 1 hour), up to minutes (e.g., up to 30 minutes, up to 12 minutes, up to about 4 minutes, up to about 2 minutes, up to about 1 minute), or seconds (e.g., up to 30 seconds, up to 5 seconds, up to 2 seconds), after mixing with the aqueous medium. The time of the tacky state may be dependent on a number of factors including relative ratio of the components, the particle sizes of the component materials, the presence of additives and the like, or the temperature of the environment. During the tacky state, the adhesive compositions will adhere to surfaces, optionally without the need for external clamping or other application of pressure. In the tacky state, the compositions will adhere bone to bone and bone to other materials. In the tacky state, the compositions may adhere materials such as stainless steel, titanium, zirconia, polyether ether ketone, steel, aluminum, copper, brass, aragonite, calcite, cement, alumina, concrete, ceramics, rock, glass, and other metals or substances. During the tacky state the contacting surfaces may be held together by the adhesive composition itself, without the need for external force, until the composition sets to the final hardened cement state. The tacky state can allow the materials to be positioned or repositioned without appreciable loss of cured strength.

In some embodiments, the adhesive compositions may adopt a pliable working or putty state after mixing with an aqueous medium prior to hardening, which is present for up to about one week or less, one day or less, one hour or less, 30 minutes or less, depending on the components of said compositions and the conditions of the application, e.g., temperature. In some embodiments, the adhesive compositions may adopt a pliable working or putty state for less than or equal to about one week after mixing with an aqueous solution or suspension, e.g., less than about six days, less than about five days, less than about four days, less than about three days, less than about two days, less than about one day, less than about twelve hours, less than about one hour, less than about 30 minutes, less than about 20 minutes, less than about 15 minutes, less than about 10 minutes, less than about 5 minutes, less than about 3 minutes, less than about 2 minutes, less than about 1 minute, less than about 30 seconds, less than about 5 seconds after mixing with an aqueous solution or suspension.

During the putty state, which follows the tacky state, the adhesive compositions can be shaped or sculpted, for example, to fill voids in bone or acquire a desired contour, size or form. The combined time of the tacky state and the putty state is referred to herein as working time. Typical compositions may have a working time of up to at least 3 minutes, up to at least 5 minutes, up to at least 8 minutes, up to at least 12 minutes, or up to at least 15 minutes from initial mixing, after which time the compositions have sufficiently begun hardening.

In some embodiments, the adhesive composition, when mixed, has a separation strength of about 10 kPa to about 150 kPa after mixing. In some embodiments, during said tacky state, the adhesive composition has a tack strength to surfaces (e.g., bone, metal, plastic) of between about 10 kPa and about 250 kPa. In some embodiments, the bone substitute has a putty state for between 10 and 20 minutes after the tacky state. In some embodiments, the adhesive composition has a putty state for about 15 minutes. In some embodiments the tack strength in the putty state is between about 10 kPa and about 250 kPa. In some embodiments, upon curing, the adhesive composition has an adhesive strength to surfaces (e.g., bone, metal, or plastic) of between 250 kPa and 1000 kPa. In certain embodiments, the adhesive composition has an adhesive strength to surfaces (e.g., bone, metal, or plastic) greater than 1000 kPa.

In some embodiments, the adhesive compositions may exhibit an adhesive strength in the cement-like state in the range of about 100 kPa to about 12,000 kPa, depending on the application and the particular components and ratios of components in said adhesive compositions. In some embodiments, the adhesive strength of the adhesive compositions in the cement-like state is between about 100 kPa and e.g., about 10,000 kPa, about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the adhesive compositions in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the adhesive compositions in the cement-like state is in the range of about 200 kPa and about 2,500 kPa. In some embodiments, the adhesive strength of the adhesive compositions in the cement-like state is greater than 100 kPa.

The adhesive compositions described herein might be applied to the surface of a structure in its fluid or semi-solid state by means of an injection delivery device or by application using an instrument such as a spatula. The viscosity of the adhesive composition when in its fluid state might be as low as about 100 cP to about 10,000 cP and when it reaches its semi-solid state from about 10,000 cP to about 250,000 cP. The viscosity and cohesion properties of the adhesive composition will facilitate the ability to squeeze the material through a needle or cannula as small as 18 gauge when the viscosity is in the low range of its fluid state. With viscosities in the semi-solid state, the shape and amount of material can be altered through spreading or removal techniques without substantially effecting the strength of the set material. In some embodiments, the working time of the adhesive composition is when the viscosity is between about 100 cP to about 250,000 cP.

The viscosity and cohesion properties of the adhesive composition will facilitate the ability to squeeze the material through a needle or cannula as small as 18 gauge when the viscosity is in the low range of its fluid state. With viscosities in the semi-solid state, the shape and amount of material can be altered through spreading or removal techniques without substantially effecting the strength of the set material.

In some embodiments, the composition comprises a therapeutic or a combination of therapeutics. For example, in some embodiments, the adhesive composition comprises a therapeutic or a combination of therapeutics. The therapeutic may be selected to treat any number of ailments or conditions (e.g., pain, infection, cancer, osteoporosis) or to help accelerate local tissue regeneration (e.g., growth hormone, bone morphogenic protein) or to assist with surgical or therapeutic treatment (e.g., imaging modality), or any of a combination thereof. In some embodiments, the therapeutic used in the composition may mitigate post-operative pain.

In some embodiments the therapeutic is a therapeutic or a combination thereof to treat pain (e.g., pain reliever). The pain reliever may include, but is not limited to, opioids, non-steroidal anti-inflammatory (NSAIDs), local anesthetics, and gabapentinoids. Opioids may include, but are not limited to, fentanyl, morphine, hydromorphine, oxycodone, vicodin, and codeine. NSAIDs (cyclooxygenase inhibitors) may include, but are not limited to, naproxen, ibuprofen, diclofenac and acetylsalicylic acid. Local anesthetics (sodium channel blockers) may include, but are not limited to, bupivacaine, lidocaine, prilocaine, and articaine. Gabapentinoids (calcium channel blockers) may include, but are not limited to, gabapentin, pregabalin, and mirogabilin.

In some embodiments the therapeutic is a therapeutic or a combination thereof to treat infection (e.g., antibiotic). The antibiotics may include, but are not limited to, penicillins (0-lactams), cephalosporins (γ-lactams), macrolides, lincomycin, nitroimidazoles, carbapenems, fluoroquinolones, sulfonamides, tetracyclines, aminoglycosides, quinolone, polyketides, and glycopeptides. Penicillins may include, but are not limited to, amixocillin. Cephalosporins may include, but not are limited to, cefazolin, cefaclor, cephalexin and ceftriaxone. Marcolides may include, but are not limited to, erythromycin, and clarithromycin. Fluoroquinolones may include, but are not limited to, ciprofloxacin, ofloxacin. Sulfonamides may include, but are not limited to, co-trimoxazole, and trimethoprim. Tetracyclines may include, but are not limited to, tetracycline, and doxycycline. Aminoglycosides may include, but are not limited to, gentamicin, and tobramycin. Glycopeptides may include, but are not limited to, vancomycin teicoplanin, telavancin, ramoplanin and decaplanin, and bleomycin.

In some embodiments, the therapeutic is a therapeutic or a combination thereof to treat cancer (e.g., chemotherapeutic). Chemotherapeutic agents may include, but are not limited to, alkylating agents, antimetabolites, antibiotics, topoisomerase, and tyrosine kinase inhibitors. The alkylating agents may include, but are not limited to, cyclophosphamide, dacarbazine, temozolomide, and aziridine. Antimetabolites may include, but are not limited to, 5-Fluorouracil, cytarabine, gemcitabine, and methotrexate. Anticancer antibiotics may include, but are not limited to epirubicin, and idarubicin. Topoisomerase may include, but is not limited to, irinotecan, camptothecin, doxorubicin, daunorubicin, and teniposide. Tyrosine kinase inhibitor may include, but is not limited to, erlotinib, sunitinib, dasatinib, and axitinib.

In some embodiments the therapeutic is a therapeutic or a combination thereof to regenerate tissue (e.g., growth hormone, bone morphogenetic protein, hormones). Bone growth adjuvants may include citric acid. Hormones may include, but are not limited to, cholecalciferol, i.e., Vitamin D₃ form.

In some embodiments, the therapeutic is a therapeutic moiety or a combination thereof to enhance imaging. Imaging agents may include, but are not limited to, gadolinium-based contrast agent, barium-based contrast agents, and iron/iron oxide-based contrast agent. In some embodiments, an imaging agent is used in conjunction with an additional therapeutic wherein the imaging agent allows for the monitoring of the controlled released of the other therapeutic agent. For example, the contrast agent is used in conjunction with a chemotherapeutic wherein the contrast agent monitors the placement of not just the adhesive composition but also the subsequent release of the chemotherapeutic from the site of composition placement.

In some embodiments, the therapeutic can be a fine powder or granular of any size range. These powders may exhibit a mean particle size of about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. In some embodiments, the powder may have a mean particle size of less than about 1.000 mm. In some embodiments, the particle size distribution may be multi-modal to include any combination of mean particle sizes as previously described. In some embodiments, the granules may exhibit a mean granule size of about 0.050 mm to about 5 mm, about 0.100 to about 1.500 mm, about 0.125 to about 1.000 mm, 0.125 to about 0.500 mm, about 0.125 to about 0.250 mm, about 0.250 to about 0.750 mm, about 0.250 to about 0.500 mm, about 0.500 to about 1.00 mm, about 0.500 to about 0.750 mm. In some embodiments, the granule size may be multi-modal to include any combination of mean granule sizes as previously described. The granules may be supplied with a various proportion of porosity and a various size of internal pores. The pores may communicate with granule surface or not. In some embodiments, varying sizes of said powders or granules may be used in the adhesive composition.

In some embodiments, the amount of a therapeutic (e.g., pain reliever, antibiotic, anticlastic drug, growth hormone, bone morphogenetic protein, chemotherapeutic, imagine modality, or a combination thereof) in the compositions may vary, e.g., between about 0.001% to about 40% weight by weight (w/w) of the total composition. In some embodiments, the amount of a therapeutic in the compositions is in the range of about 0.1% to about 40%, about 0.001% to about 1%, about 0.1% to about 1%, about 1% to about 5%, about 5% to about 10%, about 1% to about 25%, about 2% to about 10%, about 3% to about 5%, or about 20% to about 40% w/w of the total composition.

In some embodiments, the amount of the therapeutic in the composition may be between about 0.001% to about 40% weight by weight (w/w) of the total composition. In some embodiments, the amount of a therapeutic in the compositions is in the range of about 0.1% to about 40%, about 0.001% to about 1%, about 0.1% to about 1%, about 1% to about 5%, about 5% to about 10%, about 1% to about 25%, about 2% to about 10%, about 3% to about 5%, or about 20% to about 40% w/w of the total composition.

In some embodiments, the therapeutic or a combination thereof in the adhesive composition may be released. In some embodiments, the therapeutic or a combination thereof (e.g., pain reliever, antibiotic, anticlastic drug, growth hormone, bone morphogenetic protein, chemotherapeutic, imagine modality) in the composition may be controllably released from the compositions within seconds to a minute post implantation of the composition. In some embodiments, the therapeutic may be released from the composition within weeks post implantation of the composition. In some embodiments, the therapeutic may be released from the composition within months post implantation of the composition. In some embodiments, the therapeutic may be released from the composition within years post implantation of the composition. In some embodiments, release of the therapeutic agent can be monitored via an additional imaging therapeutic released from the composition. For example, an adhesive composition comprises a combination of a chemotherapeutic, e.g., antimetabolite and an imaging therapeutic, e.g., BaSO₄, wherein the combination of therapeutics is released after delivery of the composition and can be viewed via fluoroscopy of the contrast agent.

In some embodiments, the rate of release of the therapeutic or a combination thereof from the composition, e.g., adhesive composition, may be controlled. In some embodiments, the therapeutic or a combination thereof (e.g., pain reliever, antibiotic, anticlastic drug, growth hormone, bone morphogenetic protein, chemotherapeutic, imagine modality) is released at a rate from the composition that is controlled to achieve sustained release until the composition completely biodegrades or until the therapeutic is completely released from the composition. In some embodiments, the therapeutic can have initial burst release from the composition followed by a gradual release. In some embodiments, the therapeutic can have an initial slow, gradual release from the composition followed by a burst release. In some embodiments, the rate of therapeutic release or release profile of a target therapeutic can be tailored to a specific time-dose curve using conventional time release technology such as passive diffusion. The rate of therapeutic release or release profile from the composition may be optimized based on the nature of the therapeutic (acid, base or salt). In some embodiments, the rate of therapeutic release or release rate from the composition may be controlled by altering the particle size distribution of one or more components (e.g., multivalent metal, compound of a Formula, additive) of the composition, by introducing and maximizing porosity and interconnected pores into the matrix and by minimizing the therapeutic interaction with other compounds in the composition. In some embodiments, the rate of therapeutic release or release profile of a target therapeutic can be tailored to a specific time-dose curve governed by a combination of the factors described above (e.g., interaction with adhesive composition, resorption, diffusion, particle size, porosity, pore size).

The present disclosure provides that the therapeutic may be controllably released so that the therapeutic release follows a specific time-dose curve. In some embodiments, this is achieved by forming the pores during the setting process of the composition to increase the surface area of the composition and therefore the diffusion rate. More specifically, a salt additive (e.g., calcium carbonate, calcium bicarbonate, sodium carbonate, or sodium bicarbonate) is included in the composition wherein said additive reacts in the composition to form pores and porosity during the in situ hardening or curing process of the adhesive composition. More specifically, as the additive reacts with the multivalent metal salt and compound present in the composition, carbon dioxide gas is released in the form of bubbles which form pores and cause the adhesive composition to expand while hardening. The amount of additive included in the composition directly correlates to how porous the adhesive composition is. In some embodiments, the amount of this additive in the composition may range from about 0.5% to about 20% w/w of the solid components of the adhesive composition. In some embodiments, the porosity of the adhesive composition ranges from about 5% to about 95% in direct correlation to the amount of additive present in the composition. The therapeutic present in the adhesive composition is able to move diffusely through the pores and to the defect site or throughout the body.

In some embodiments, the method of controlling the release of the therapeutic comprises including porous granules as an additive of the adhesive composition wherein said porous granules consist of a solidified form of the adhesive composition wherein said pores are created in the same method as described above. In some embodiments, said granules comprise an amount of the therapeutic and said therapeutic is released through the granule additives at the defect sites. In some embodiments, porous granules are present in addition to the adhesive composition itself being porous via the method described above, wherein the therapeutic is released from both the porous composition and the porous granule additives.

In some embodiments, the release rate of the therapeutic out of the composition can be determined by the rate of composition biodegradation, wherein the release rate of the therapeutic follows a linear model along with the biodegradation or bioabsorbtion of the adhesive composition. In other embodiments, the release rate of the therapeutic out of the composition is determined by the partition coefficient of the specific therapeutic. A partition coefficient is a measurement of the affinity of a therapeutic to the composition, i.e., the ratio of the amount of drug released from the composition to the ratio of the amount of drug retained in the composition under specific positions. The higher the partition coefficient is, the shorter the release time is. The partition coefficients of various therapeutics were calculated in Example 2.b below and can be found in Table 3.

In some embodiments, the therapeutic is present in the adhesive composition as an additive wherein: the therapeutic is mixed into a solidified form of the adhesive composition via the method described above; said solidified form is preformed during the putty state into a preferred state (e.g., disc, plug, cylinder, rod, bar, mesh, etc.); and said preformed composition is surgically implanted into or onto the defect site (e.g., bone or bone surface). Said preformed composition may be affixed in place via a compression or wedge fit, with an additional amount of non-preformed adhesive composition, using hardware (e.g., screws), or any combination thereof. In some embodiments, the preformed composition could be in a predefined geometric shape for a specific application, or made a custom size based on a patient specific anatomic site.

The adhesive composition, including a therapeutic, is capable of undergoing sterilization, e.g., gamma irradiation, except wherein certain therapeutics may not be capable of undergoing gamma irradiation where sterilization would degrade the chemical structure of the composition. Sterilization may not compromise the chemical structure of the composition, determined by shifts in the chemical structure of the composition. Said compositions are capable of undergoing gamma irradiation up to 50 kGy, preferably between 10 and 40 kGy, or between 20 and 30 kGy.

In some embodiments, the disclosure features a structure comprising (e.g., formed from) an adhesive composition. In some embodiments, said structure comprises a solidified form of an adhesive composition. The structure may be substantially comprised of the adhesive composition, or may comprise additional components (e.g., a fiber). Exemplary bone segments may comprise an additional layer of the adhesive composition (e.g., in the working state) as a coating on the surface of the structure or impregnated into or onto the surface of the adhesive structure or into the kerf space where the bone segment is placed into is original or corrected position. In some embodiments, an additional layer of the adhesive composition is partially or fully filled into the kerf space to fixate the bone segment. In some embodiments, the bone segment and an additional layer of the adhesive composition is used to block the flow of an aqueous medium. In some embodiments, the bone segment and an additional layer of the adhesive composition is used to reinforce a structure (e.g., cranium or spine). In some embodiments, the bone segment and an additional layer of the adhesive composition is used to join separated objects. In other embodiments, the bone segment and an additional layer of an adhesive composition is used for filling of space to connect and immobilize a structure. In still other embodiments, the bone segment and an additional layer of an adhesive composition may be used in a method of treating a subject suffering from a disease or condition.

### Kits

The present disclosure further provides a kit for use in a procedure as described herein. In some embodiments, a kit includes a device as disclosed herein and an adhesive composition as described herein. In some embodiments, said kit includes at least two of the same device, e.g., two RFDs or two HFDs. In some embodiments, the kit includes at least two of each device, e.g., at least two RFDs and at least two HFDs. In some embodiments, any number of components of the devices are single-use and disposable. As such, a kit may include a number of device bases and a variety of disposable device components to be attached for a single procedure and then disposed of. In some embodiments, the kit includes a disposable part of a device, such as one or more retaining elements RE of the RFD or HFD. In this configuration, the one or more REs are in patient contact and thus disposed of following fixation and reusable components of the RFD or HFD, e.g., the base elements BE, are capable of being used for another fixation procedure. In some embodiments, the kit includes elastic bands or coils having different stretch characteristics which are color-coded or otherwise labelled to reflect their behavior once applied to the system. In some embodiments, the kit further comprises a wire, e.g., a k-wire or intra-oral wire, for providing further stabilization of a bone segment or structure while an adhesive composition, e.g., the adhesive composition as described herein, cures.

In some embodiments, the adhesive composition, e.g., as described herein, or another adhesive composition preselected by the user, is packaged separately from the device. In some embodiments, the adhesive composition is provided in a sterile package within the kit. In some embodiments, the adhesive composition is provided in a syringe within the kit. In some embodiments, there are multiple sterile packages comprising the chosen adhesive composition, e.g., for use in a complex fracture requiring multiple applications. In some embodiments, the components of the adhesive composition are provided separately within the sterile packaging such that they are activated upon use and application. In some embodiments, one or more adhesive compositions having different properties are provided within the kit such that the user is able to choose which to use. In some embodiments, the user is able to use third party compositions with the devices provided in said kit.

### Methods of Use

The present disclosure features methods of use, e.g., methods of positioning, for of the devices and compositions described herein. The range of applications of the device embodiments and methods of use described herein may include: adhesive repair and stabilization of bones, fracture treatment, repair and stabilization following ostectomy of diseased bone tissue, e.g., fibrous dysplasia, Paget's disease, and other similar indications. In some embodiments, structures subject to a procedure as described herein include: bone subject to osteotomy, e.g., tibial osteotomy for treatment or prevention of osteoarthritis, articular joints requiring fusion, e.g., ankle fusion, first metatarsophalangeal joint fusion, wrist fusion, damaged or diseased joints requiring arthroplasty component alignment or stabilization, e.g., shoulder joint, wrist joint, first metacarpophalangeal joint, hip joint, knee joint, ankle, or a first metatarsophalangeal joint, instability of bone created by surgical procedure, e.g., sternotomy, while longer term stabilization method, e.g., wiring, stapling, or plating, is used, and/or to treat conditions where a gap defect exists following partial bone resection, e.g., for removal of tumor, fibrous union resection, or elective bone length resizing.

In some embodiments, the devices and methods of use described herein may improve cosmetic appearance by one or more of: increased level positioning of the bone segments, prevention of tissue sagging into the osteotomy, i.e., burr holes or kerf lines, or by elimination of hardware protrusion, e.g., resting above the bony contour. In some embodiments, elimination of hardware protrusion reduces palpability and soft tissue irritation over time, and therefore causes reduction in pain. In some embodiments, the devices and methods of use described herein create a watertight seal of the osteotomy, i.e., burr holes or kerf lines, and thereby aide in the prevention of hydrodynamic complications e.g., leaks of cerebrospinal fluid out of, or bacterial penetration into, the cranial cavity. The creation of a watertight seal reduces the occurrence of secondary infections.

In some embodiments, the devices and methods of use described herein improve the fixation strength of the bone segments and relative motion of the bone segments as compared the current standard of care, e.g., plates and screws, by providing an adhesive composition that solidifies into a structural scaffold within the kerf or space that bonds the bone segments together. In some embodiments, said structural scaffold may be comprised of a resorbable biomaterial and replaced by native material over time. In some embodiments, the devices, adhesive compositions, and methods of use described herein create a seal around the osteotomy, i.e., burr holes and kerf lines, which can prevent fibrous tissue ingrowth. In some embodiments, the seal facilitates bone fusion, e.g., wherein the adhesive composition is bioresorbable, which provides the bone segments with a vascular supply and therefore prevents resorption in size of the bone segments, e.g., change in thickness and width. In some embodiments, the devices, adhesive compositions, and methods of use described herein improve placement, improve fixation strength and prevent migration of the bone segments.

In some embodiments, the means of definitive fixation of bone segments or other structures as described herein do not require use of hardware, e.g., plates and screws, and thus reduce the occurrence of secondary infections by the elimination of such hardware. In some embodiments, the devices and methods of use described herein may eliminate or significantly reduce CT or MRI artifact by the elimination of metal hardware.

In some embodiments, the proportions of the devices described herein may be constructed and arranged, i.e., sized and shaped, for methods of use directed to specific bones and surgical sites. For example, retaining elements RE may comprise arms A constructed and arranged of reaching both palmar and dorsal surfaces of a metacarpal simultaneously, or of reaching the medial and lateral aspects of the calcaneus, or of reaching the superior and inferior aspects of the spine or scapula during repairs involving the coracoid process or acromion process. In some embodiments, changing the length and/or shape of the arms A using a suitable mechanism, e.g., telescoping arms, aids in the procedure by allowing the user more maneuverability of the device. In some embodiments, tines T are removable from arms A and one or more of a selection of various shaped and sized tines T are included in the device kit. In some embodiments, one set of tines T is used throughout the entire procedure. In some embodiments, one set of tines T is used and then removed and replaced with another preselected set of tines T. In some embodiments, the set of tines T used at one time vary in size and shape. For example, in a procedure open to one aspect, e.g., dorsal or ventral, but closed to the other of an anatomical structure, e.g., a femoral fracture, which is invested with thick muscle opposite the access opening.

The present disclosure provides that the base element B is capable of translational motion. In some embodiments, the motive force for translation of each base element B is provided mechanically by an operator's hand. In some embodiments, the motive force for translation is provided by elastic rebound. Said rebound force may be exerted by an elastic band, a coil spring, a leaf spring, or any similar such mechanism. In some embodiments, the retaining elements RE move relative to each other. In some embodiments, the translational force is applied to tissue structures, resulting in tissue deformation within a desired space between the structures. In some embodiments, a translational force is applied to distract structures which are attached to retaining elements RE, to allow for delivery of an adhesive composition into the gaps thus created. In some embodiments, the translational force is used to compress an adhesive composition into the gap between structures to be fixated. In some embodiments, the translational compressive force results in substantially constant compression of an adhesive composition while limiting significant distraction or other significant movement of structures. In some embodiments, translation of retaining elements RE(s) is accomplished through movement of a worm screw mechanism. In some embodiments, two or more base elements B are in contact with a single worm screw to allow coordinated movement of base elements B relative to one another and to the rail component R. In some embodiments, translational movement of base elements B is irreversibly or reversibly locked to prevent relative movement of each retaining element RE thus retaining the device in a desired position during the procedure. Said immobilization of REs along the rail may be accomplished by mechanical means, e.g., a cam mechanism, compression from a screw, a notch, a movable wedge jam, or with other mechanical features. In some embodiments, irreversible immobilization of each base element B relative to the rail R is accomplished with the use of a rapidly setting cement, e.g., cyanoacrylate. In some embodiments, immobilization of each base element B relative to the rail R is accomplished with use of a crimp or swage deformation of device structure. In some embodiments, immobilization of the base element B relative to the rail R is accomplished by use of a ratchet, latch, or hasp mechanism. Any embodiment disclosed herein may include one, two, three or more independent mechanisms for immobilization.

The present disclosure further provides for use of a wire component, e.g., a k-wire or a custom intra-oral wire, in conjunction with the devices described herein. In some embodiments, a custom intra-oral wire is provided as a component of the kit wherein said intra-oral wire provides enhanced stabilization to an oral procedure and minimizes tensile stress on the applied adhesive composition while it cures. In some embodiments, the use of a wire as an additional component of the devices described herein acts as a retaining feature. In some embodiments, said wire is removable and disposable.

The present disclosure further provides methods of use of one or more hinged fixation devices HFDs. In some embodiments, the fractured bone is reduced to the proper anatomical relationship and the HFD is applied to this relationship, locking all axes of motion except for translation, thus leaving only the hinge articulation unlocked. In some embodiments, one HFD is applied to one structure, and another HFD is applied to another structure. In some embodiments, there are two, three, or more structures, e.g., bone fragments or segments, to be fixated. In some embodiments, the bone segment surfaces are then separated along the translational axis to allow placement of an exemplary adhesive composition. In some embodiments, the bone segments are then reapproximated to their preferred relative positions. In some embodiments, a compressive force is then applied to the bone segments to allow the adhesive composition to cure and set. In some embodiments, two hinged fixation devices HFDs are utilized to temporarily fixate structures, e.g., bone fragments, segments, etc., while an adhesive composition is delivered to said structures to cause definitive fixation. In some embodiments, each HFD temporarily fixates a separate structure, e.g., bone fragment or segment. In some embodiments, each HFD temporarily fixates part of one or more structures.

In some embodiments, a method of use of two or more HFDs comprises: attaching a hinged fixation device HFD to each bone segment or structure to be fixated in a manner that maintains the preferred assembly or spatial relationship of structures, e.g., bone segments, bone fragments or regenerative biocompatible materials, reversibly separating these structures along an allowed degrees of freedom of relative motion, e.g., rotational or translational, to allow application of an adhesive composition, e.g., as described herein, to the gap between the structures, and guiding the reassembly and alignment of the structures along the allowed degrees of freedom into the predetermined preferred spatial relationship, under a controlled lever of compression while preventing unwanted motion during the curing and setting of the adhesive composition, or placement of other permanent means of fixation. In some embodiments, engagement of retaining elements RE to the structure, e.g., bone segment or fragment, includes modification of bone surface through the formation of a recess or dimple to allow penetration of the original bone contour by contact points CP or tines T in order to prevent slippage of the structure, e.g., bone fragment or segment, relative to the device in use. In some embodiments, each retaining element RE of each hinged fixation device HFD utilizes elastic rebound force applied to contact points CP on the structures to be fixated, e.g., bone fragments or bone segments, to maintain a stable and unique spatial relationship without slippage of the structures. In some embodiments, the magnitude of elastic rebound force at each contact point CP is in the range of about 2 to about 250 N, e.g., approximately 2-10 N, approximately 10-50 N, approximately 50-100N, or approximately 100-250N. depending on the application in which the device is being engaged, e.g., density of bone contacted, the strength of the structure, the magnitude of dislodging forced anticipated, etc. For example, small bone repair applications may require lower force than larger bone repair applications, or applications where the bone is surrounded by soft tissue layers, or unfavorable muscle tonus. In some embodiments, the source of elastic rebound force is tensile force from a stretched elastic band, a coil spring, a leaf spring, or another similar mechanism. In some embodiments, the point of application of this elastic force mechanism is located between the arms A of the retaining element RE. In some embodiments, the magnitude of force applied to contact points CP is controlled and preset by elective engagement of perforations within the elastic band or other rebound force mechanism. In some embodiments, the magnitude of force applied to contact points CP is controlled and preset by selection of an elastic band with a more or less stiff stretch characteristic. For example, the stretch characteristics may vary based on the thickness of the band, the length of the band in its relaxed state, or the modulus of the material comprising the band. In some embodiments, the magnitude of force applied to CP is controlled and set by the number of elastically deformable, rebounding elements, e.g., elastic bands applied. In some embodiments, the stretch characteristic is varied based on the stiffness of the coil selected. In some embodiments, the source of elastic rebound force is the relaxation rebound of elastic bending strain on the arms A. In some embodiments, the magnitude of force, e.g., grasping force, applied to the structure, e.g., bone segment, is constant and preset by the amount of elastic deformation of A by a ratchet mechanism, a screw, a catch, or another similar mechanism.

In some embodiments, the magnitude of force used for grasping each bone segment or structure by the retaining elements RE or tines T is between 20 N and 200 N. In some embodiments, the magnitude of force depends on the size and shape of the bone segments or structures being grasped, the method of grasping, and the number of contact points CP. In some embodiments, the magnitude of grasping force is maintained at a substantially constant level by a mechanism, e.g., as described herein.

In some embodiments, the retaining element RE surface supports the structure to be fixated, e.g., bone segment, without grasping it by the means of sharp contact points CP, in a desired spatial relationship with the other structures at the surgical site. In some embodiments, retaining element RE comprises a standardized interface, e.g., threaded element, friction-retained element, anti-rotational channel element, or another similar mechanism, which accommodates the attachment of a standardized or customizable structure, e.g., a preformed shape comprising a synthetic or a regenerative biomaterial which is complementary to the retaining element RE in form and size. In some embodiments, some components of the device are capable of more than one function or the functionalities of various exemplary components are performed by just one component in application. For example, the functionalities of the retaining element base B, bracing element BE, and translational stop S may be performed by a single physical component capable of electively translating along the rail and possessing an interface for the bracing element BE and retaining element connector C. In some embodiments, said bracing element BE interface is substantially the same as C interface. In some embodiments, there are a plurality of interfaces on a multifunctional base. In some embodiments, said multifunctional base also acts as an anchor point for attachment of a component which provides elastic force, e.g., a rubber band or a coil, by using the same interfaces as for retaining elements RE and bracing elements BE to power the translatory motion within the device construction. In some embodiments, the magnitude of force powering the translation of the bases B to and from each other is variable by changing the distance between the bases, thus increasing the elastic strain. In some embodiments, the magnitude of force applied between a base B and the rail R to which it is coupled, or between different bases B is between about 0.1N and about 100N, e.g., between about 0.1N and 1N, between about 1N and ION, or between about ION and 100N. The forces powering the translation of the bases B and the retaining elements RE coupled to them resulting in either separation or reapproximation of the structures retained by the retaining elements RE may be applied as tensile, compressive, bending, or torsional forces relative the source of the force, e.g., the elastically deformed member.

The present disclosure further provides a method of use of one or more rail fixation devices RFDs to align and fixate two or more bone segments or structures. Such fixation may be of a complex fracture, e.g., an intra-articular fracture, e.g., a fracture of the epiphysis region extending into the articular cartilage. For example, rail fixation devices RFDs may be used in the reconstruction of a long bone, e.g., a femur, tibia, radius, etc. In some embodiments, each bone segment or structure, is engaged with a separate retaining element RE via tines T which grasp the structure. In some embodiments, retaining element RE grasps each bone segment or structure via three or more grasping tines T. In some embodiments, once retaining elements RE are firmly engaged with connectors C in an unlocked state the corresponding bases B are fixated onto the rail R in their preselected translational path, wherein the rail R is approximately parallel to the structure. In some embodiments, base elements B are attached to the rail R prior to grasping the two or more bone segments or structures. In some embodiments, the bone segments or structures are then aligned into their proper, preferred, or chosen spatial relationship, e.g., relative to one another, via movement of the retaining element RE connected to each structure via translation of the bases B on a shared rail R, or movement of each retaining element RE via a joint or hinge to affect lateral movement. In some embodiments, once the structures are in their preferred locations, the retaining element RE connectors C are locked through immobilization of their joints, e.g., ball-and-socket joint, hinge, or telescope joint, via a mechanical method, e.g., a lock or screw mechanism, or via chemical means, e.g., a cyanoacrylate adhesive. In some embodiments, mechanical methods of immobilizing connectors C also include crimping, swaging or other plastic deformation. In some embodiments, rail R is located in the surgical field such that it does not interfere with the fixation and adhesive composition delivery and setting. In some embodiments, there may be bone fragments or segments that are not capable of being retained by the devices described herein because of their size or shape. Under these conditions, said segments may be isolated away from the fracture site and then reassembled in their preferred, former, or anatomical spatial relationship to each other and to the device-retained proximal and distal fragments to confirm that the realignment and/or reconstruction of the bone form is correct using imaging, e.g., fluoroscopy or radiographs. In some embodiments, the translational axis position of base element B is then recorded and then each B is translated away from each other such that each structure, e.g., bone fragment or segment is distracted from each other and an adhesive composition, e.g., as described herein, is delivered or applied between the structures. In some embodiments, once the adhesive composition is applied to the gap between the structures, or directly onto the structures themselves, the structures, e.g., bone segments, are then returned to their preferred, preset, or chosen appropriate anatomical positions, thus reestablishing the spatial relationship between the structures. In some embodiments, reestablishing the spatial relationship of the structures to be fixated, e.g., bone segments, includes returning base elements B to their previously recorded positions along rail R via translation of each base element B back toward each other.

In some embodiments, where there exist bone fragments or segments unable to be retained by RFD, said segments can be replaced back into their preferred, preset, chosen, or anatomical positions either before each base B is translated back to its previously recorded position, at the same time, or following the realignment of the structures being retained. In some embodiments, said unretained structures, e.g., large or irregular bone fragments or segments, are supported, e.g., propped up, in their desired, preferred, or chosen positions with the assistance of a component of the device, such as an arm A of the retaining element RE, or another appropriate nongrasping retaining feature of the device, e.g., a convex, flat or concave surface capable of immobilizing the structure under the conditions stated. After sufficient time has passed to allow the adhesive composition to set and cure, each device in use may be removed. In some embodiments, each retaining element RE is disengaged from the structure, e.g., bone fragment, segment, implant, or regenerative material block. In some embodiments, the strength of the adhesive composition is sufficient to support the structures, e.g., bone segments, at the fracture or injury site.

The present disclosure further provides a method of use of one or more hinged fixation devices HFD comprising a rotary hinge-type linkage arm RHLA and a twin ball joint-type linkage arm TBLA. FIGS. 8A-8R depict this method of use. In some embodiments, the first step in a method of use of such devices includes a first structure, e.g., bone segment or bone fragment being retained by the hinged linkage arm RHLA, as seen in FIG. 8B and a second structure, e.g., bone segment or bone fragment being retained by the twin ball joint linkage arm TBLA, as seen in FIG. 8C. In some embodiments, the retained structures are then positioned in a preferred anatomical alignment, e.g., by movement of the linkage arms LA, as seen in FIGS. 8D-8F. In some embodiments, once the structures are aligned, the TBLA is locked in position via a mechanical locking feature, e.g., a knob K. In some embodiments, wherein fixation hardware, e.g., a bone plate, is being used, holes for attachment of the fixation hardware are marked and drilled, as seen in FIG. 8I. In some embodiments, fixation hardware is then attached to the first structure, as seen in FIG. 8J. In some embodiments, the structures are then separated while maintaining their spatial relationship, e.g., by movement of the RHLA, as seen in FIGS. 8K-8L. In some embodiments, an adhesive composition is applied to the adjacent sides of each structure, as seen in FIG. 8M. In some embodiments, the structures are then repositioned back into their preferred anatomical alignment, as seen in FIGS. 8N-8O. In some embodiments, a compressive force is applied to the structures, as seen in FIG. 8P, and the fixation hardware, e.g., bone plate, is attached to the second structure, as seen in FIG. 8Q. In some embodiments, RHLA is then locked, e.g., via knob K, to prevent movement of the structures while the adhesive composition cures. In some embodiments, the fixation hardware, e.g., bone plate, remains in place until the adhesive composition cures, e.g., between 30 minutes to 24 hours, as seen in FIG. 8R, at which point the hardware is removed. In some embodiments, the fixation hardware remains in place after the adhesive composition has cured.

In some embodiments, a non-invasive means of stabilization, e.g., k-wire or additional application of adhesive composition, is applied to the fracture or injury site once the devices have been removed. For example, where the structure to be fixated is a tooth or dental implant, an additional application of adhesive composition may be applied across the structure and the adjacent teeth to provide secondary support. Alternatively, a k-wire may be wrapped around the structure and adjacent stabilized bone to provide secondary support.

The above methods of use as described herein may be used solely or in combination, or possibly in addition to other methods of use.

### ENUMERATED EMBODIMENTS

1. A device for positioning two or more segments or structures, the device including:
   one or more base elements;
   a plurality of retaining elements, each retaining element having a first end operatively coupled to one of the one or more base elements and a second end constructed and arranged to hold at least one of the segments or structures; and
   a component for providing positioning to the two or more segments or structures.
2. A device for positioning two or more segments or structures, the device including:
   one or more base elements;
   a plurality of retaining elements, each retaining element having a first end operatively coupled to one of the one or more base elements and a second end constructed and arranged to hold at least one of the segments or structures; and
   a rail component for providing positioning to the two or more segments or structures.
3. A device for positioning two or more segments or structures, the device including:
   one or more base elements;
   a plurality of retaining elements, each retaining element having a first end operatively coupled to one of the one or more base elements and a second end constructed and arranged to hold at least one of the segments or structures; and
   a hinge component for providing positioning to the two or more segments or structures.
4. The device of embodiments 1 or 2, wherein the rail component is rectilinear.
5. The device of any of embodiments 1, 2, or 4, wherein the rail component is curved or includes offsets.
6. The device of any of embodiments 1, 2, and 4-6, wherein the rail component includes one or more portions to achieve a combination of rectilinear and curved segments.
7. The device of any of embodiments 1, 2, and 4-7, wherein the plurality of retaining elements are attached to the rail element via the one or more base elements.
8. The device of any of embodiments 1-7, wherein the one or more base elements include a hinge which terminates in one or more arms for holding or grasping the bone segments or structures to be fixated.
9. The device of any of embodiments 1-8, wherein the one or more retaining elements include a first end constructed and arranged to translate along a length of one of the one or more arms constructed and arranged to hold one of or part of one of the two or more segments or structures to be fixated.
10. The device of embodiment 9, wherein the one or more retaining elements include a second end slidably coupled along a length of one of the one or more arms constructed and arranged to hold one of or part of one of the two or more segments or structures to be fixated.
11. The device of any of embodiments 1-10, wherein each retaining element includes a plurality of opposing sharp points constructed and arranged to penetrate soft tissue and engage bone.
12. The device of any of embodiments 1-10, wherein each retaining element includes a plurality of opposing edges constructed and arranged to penetrate soft tissue and engage bone.
13. The device of any of embodiments 1-12, wherein each retaining element includes a surface constructed and arranged to support the two or more segments or structures.
14. The device of embodiment 13, wherein the surface includes a cylindrical concave surface.
15. The device of embodiment 13, wherein the surface includes a conical concave surface.
16. The device of embodiment 13, wherein the surface includes a flat surface.
17. The device of embodiment 13, wherein the surface includes a convex surface.
18. The device of any of embodiments 1-17, wherein the device further includes a hinge.
19. The device of any of embodiments 1-18, wherein the device further includes a ball-and-socket joint.
20. The device of any of embodiments 1-19, wherein the device further includes a double ball-and-socket joint, e.g., as illustrated in FIG. 7A.
21. The device of any of embodiments 1-20, wherein the device further includes a telescopic member.
22. The device of any of embodiments 1-21, wherein the device further includes a rail with sliding bases.
23. The device of any of embodiments 1-22, wherein the connection between each of the plurality of base elements and the plurality of retaining elements is constructed and arranged to be locked to a position.
24. The device of any of embodiments 1-23, wherein each of the plurality of base elements further includes a protrusion adapted to connect to an external support.
25. The device of any of embodiments 1-24, wherein the rail component further includes one or more stops along its length constructed and arranged to limit translation of the plurality of base elements.
26. The device of any of embodiments 1-25, wherein each of the plurality of retaining elements includes one or more contact points for contacting or grasping the two or more segments or structures.
27. The device of embodiment 26, wherein each of the one or more contact points includes one or more tines.
28. The device of embodiment 26 or 27, wherein the one or more contact points are disposed orthogonal to one or more arms of the plurality of retaining elements.
29. The device of any of embodiments 1-28, further including handles having a length that are constructed and arranged to be connectable to each of the plurality of retaining elements.
30. The device of embodiment 29, wherein the handles are detachable from the positioning device.
31. The device of any of embodiments 1-28, wherein the device further includes a first linkage arm including a hinge and one or more retaining elements.
32. The device of embodiment 31, wherein the device further includes a second linkage arm including at least one ball joint and one or more retaining elements, the first and second linkage arms constructed and arranged to be independently positioned to hold at least one of the segments or structures to be fixated.
33. A method of positioning two or more segments or structures, including:
   prepositioning the two or more segments or structures in an initial position;
   attaching at least one positioning device to each of the two or more segments or structures;
   distracting the two or more segments or structures such that a gap is created between each of the two or more segments or structures;
   applying a composition to the gap between the two or more segments or structures; and
   repositioning the two or more segments or structures with the applied composition to the initial position, thereby positioning the two or more segments or structures as the composition cures.
34. The method of embodiment 33, wherein the two or more segments or structures to be positioned include bone segments.
35. The method of embodiment 33, wherein the two or more segments or structures to be positioned include bone fragments.
36. The method of embodiment 33, wherein the two or more segments or structures to be positioned include other biologically compatible structures.
37. The method of any of embodiments 33-36, wherein the positioning device includes: one or more base elements;
   a plurality of retaining elements, each retaining element having a first end operatively coupled to one of the one or more base elements and a second end constructed and arranged to hold at least one of the segments or structures; and
   a component for providing positioning to the two or more segments or structures.
38. The method of any of embodiments 33-36, wherein the positioning device includes:
   one or more base elements;
   a plurality of retaining elements, each retaining element having a first end operatively coupled to one of the one or more base elements and a second end constructed and arranged to hold at least one of the segments or structures; and
   a rail component for providing positioning to the two or more segments or structures.
39. The method of any of embodiments 33-36, wherein the positioning device includes:
   one or more base elements;
   a plurality of retaining elements, each retaining element having a first end operatively coupled to one of the one or more base elements and a second end constructed and arranged to hold at least one of the segments or structures; and
   a hinge component for providing positioning to the two or more segments or structures.
40. The method of any of embodiments 33-39, wherein the rail component is disposed substantially parallel to the two or more segments or structures.
41. The method of any of embodiments 33-40, wherein the hinge component is attached to a base element of the device and includes one or more arms for grasping the two or more segments or structures.
42. The method of any of embodiments 33-41, wherein the one or more base elements are slidably coupled to and disposed along a length of the rail component.
43. The method of any of embodiments 33-42, wherein each of the plurality of retaining elements grasps one of the two or more segments or structures.
44. The method of any of embodiments 33-43, wherein each of the plurality of retaining elements grasps a portion of the two or more bone segments or structures.
45. The method of any of embodiments 33-44, wherein each of the plurality of retaining elements includes a plurality of opposing points for grasping and holding the two or more segments or structures constructed and arranged to penetrate soft tissue and engage bone.
46. The method of any of embodiments 33-44, wherein each of the plurality of retaining elements includes a plurality of opposing edges for grasping and holding the two or more segments or structures constructed and arranged to penetrate soft tissue and engage bone.
47. The method of any of embodiments 33-46, wherein each of the plurality of retaining elements includes a surface constructed and arranged to support the two or more segments or structures.
48. The method of any of embodiments 33-47, wherein the surface includes a cylindrical concave surface.
49. The method of any of embodiments 33-47, wherein the surface includes conical concave surface.
50. The method of any of embodiments 33-47, wherein the surface includes a flat surface.
51. The method of any of embodiments 33-47, wherein the surface includes a convex surface.
52. The method of any of embodiments 33-51, wherein the coupling between each of the plurality of base elements and the plurality of retaining elements is constructed and arranged to be fixed into a position.
53. The method of any of embodiments 33-52, wherein positioning the two or more segments or structures includes positioning the base elements of the positioning device to limit the motion of the two or more segments or structures to one translational degree of freedom.
54. The method of any of embodiments 33-53, wherein positioning the two or more segments or structures includes positioning the base elements of the positioning device to limit the motion of the two or more segments or structures to one rotational degree of freedom.
55. The method of any of embodiments 33-54, wherein positioning the two or more segments or structures includes positioning the base elements of the positioning device to limit the motion of the two or more segments or structures to one translational and one rotational degree of freedom.
56. The method of any of embodiments 33-55, wherein the limiting of the motion of the two or more segments or structures is provided mechanically.
57. The method of embodiment 56, including limiting of the motion of the two or more segments or structures by applying a compressive force to the plurality of retaining elements.
58. The method of embodiment 56 or 57, including limiting of the motion of the two or more segments or structures by crimping or swage deformation.
59. The method of any of embodiments 33-58, including maintaining the positioning of the two or more segments or structures using an applied force.
60. The method of embodiment 59, wherein the force applied is between 20 N to 150 N.
61. The method of any of embodiments 33-60, wherein the limiting of the motion of the two or more segments or structures is provided chemically.
62. The method of embodiment 61, including limiting of the motion of the two or more segments or structures by application of a cyanoacrylate cement to one or more components of the positioning device.
63. The method of any of embodiments 33-62, wherein positioning the two or more segments or structures includes further includes actuating a hinge operatively coupled to the one or more retaining elements.
64. The method of any of embodiments 33-63, wherein each of the plurality of base elements of the positioning device further includes a protrusion adapted to connect to an external support.
65. The method of any of embodiments 33-64, wherein the rail component of the positioning device further includes one or more stops along its length arranged to limit translation of the plurality of base elements.
66. The method of any of embodiments 33-65, wherein each of the plurality of retaining elements of the positioning device includes one or more contact points for maintaining contact with or grasping each of the two or more segments or structures.
67. The method of any of embodiments 33-66, wherein the position device further includes handles having a length that are constructed and arranged to be connectable to each of the plurality of retaining elements.
68. The method of embodiment 67, wherein the handles are detachable from the rest of the positioning device.
69. The method of embodiment 67 or 68, wherein the handles are detached once the two or more segments or structures have been positioned and fixated
70. The method of any of embodiments 67-69, wherein the handles reduce stress or strain on the two or more segments or structures during the curing of the composition while the positioning device is attached to the two or more segments or structures.
71. The method of any of embodiments 33-70, wherein repositioning the two or more segments or structures further includes applying a compressive force to composition-bearing surfaces of the two or more segments or structures to be fixated.
72. The method of any of embodiments 33-71, wherein the composition is an adhesive composition.
73. The method of any of embodiments 33-72, wherein the composition is a bone restorative composition.
74. The method of any one of the preceding embodiments, wherein the composition comprises a multivalent metal salt, an organic compound, and an aqueous medium.
75. The method of embodiment 74, wherein the multivalent metal salt comprises one or more of calcium phosphates (e.g., hydroxyapatite, octacalcium phosphate, tetracalcium phosphate, tricalcium phosphate), calcium nitrate, calcium citrate, calcium carbonate, magnesium phosphates, sodium silicates, lithium phosphates, titanium phosphates, strontium phosphates, barium phosphates, zinc phosphates, calcium oxide, magnesium oxide, and combinations thereof.
76. The method of embodiment 74 or 75, wherein the multivalent metal salt comprises tetracalcium phosphate.
77. The method of embodiment 74 or 75, wherein the multivalent metal salt comprises tricalcium phosphate.
78. The method of embodiment 77, wherein the multivalent metal salt comprises α-tricalcium phosphate.
79. The method of embodiment 74 or 75, wherein the multivalent metal salt comprises (3-tricalcium phosphate.
80. The method of embodiment 74 or 75, wherein the multivalent metal salt comprises hydroxyapatite.
81. The method of embodiment 74 or 75, wherein the multivalent metal salt comprises octacalcium phosphate.
82. The method of embodiment 74 or 75, wherein the multivalent metal salt comprises calcium nitrate.
83. The method of embodiment 74 or 75, wherein the multivalent metal salt comprises calcium citrate.
84. The method of embodiment 74 or 75, wherein the multivalent metal salt comprises calcium carbonate.
85. The method of embodiment 74 or 75, wherein the multivalent metal salt comprises magnesium phosphates.
86. The method of embodiment 74 or 75, wherein the multivalent metal salt comprises sodium silicates.
87. The method of embodiment 74 or 75, wherein the multivalent metal salt comprises lithium phosphates.
88. The method of embodiment 74 or 75, wherein the multivalent metal salt comprises titanium phosphates.
89. The method of embodiment 74 or 75, wherein the multivalent metal salt comprises strontium phosphates.
90. The method of embodiment 74 or 75, wherein the multivalent metal salt comprises barium phosphates.
91. The method of embodiment 74 or 75, wherein the multivalent metal salt comprises zinc phosphates.
92. The method of embodiment 74 or 75, wherein the multivalent metal salt comprises calcium oxide.
93. The method of embodiment 74 or 75, wherein the multivalent metal salt comprises magnesium oxide.
94. The method of any one of embodiments 74-93, wherein the organic compound is a compound of any one of Formulas (I), (II), (III), (IV), (V), or (VI).
95. The method of embodiment 94, wherein the organic compound is a compound of Formula (I):

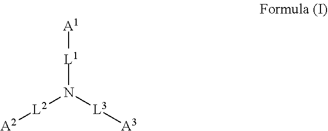

or a salt thereof, wherein:
each of A¹, A², and A³ is independently selected from an acidic group; and
each of L¹, L², and L³ is independently a bond, alkylene, or heteroalkylene.
96. The method of embodiment 95, wherein each of A¹, A², and A³ is independently a carboxyl or phosphonyl.
97. The method of embodiment 95, wherein A¹ is carboxyl, and A² and A³ are phosphonyl.
98. The method of embodiment 95, wherein A¹, A² and A³ are phosphonyl.
99. The method of any of embodiments 95-98, wherein each of L¹, L², and L³ is C₁-C₃ alkylene.
100. The method of embodiment 99, each of L¹, L², and L³ is C₁ alkylene.
101. The method of any of embodiments 95-99, wherein the compound of Formula (I) is a compound of Formula (I-a):

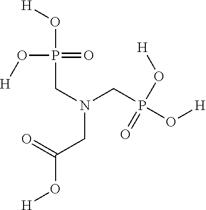 [SMILES: O=C(O)CN(CP(=O)(O)O)CP(=O)(O)O]

102. The method of any of embodiments 95-99, wherein the compound of Formula (I) is a compound of Formula (I-b):

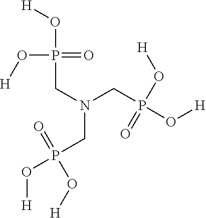 [SMILES: O=P(O)(O)CN(CP(=O)(O)O)CP(=O)(O)O]

103. The method of embodiment 94, wherein the organic compound is a compound of Formula (II):

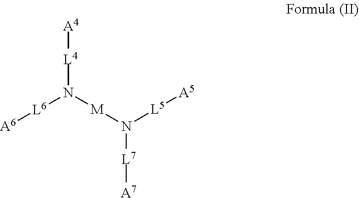

or a salt thereof, wherein:
each of A⁴, A⁵, and A⁶, is independently selected from an acidic group;
A⁷ is selected from an acidic group, a hydrogen atom, an alkyl, an aryl, a hydroxy group, a thio group, and an amino group;
each of L⁴, L⁵, L⁶, and L⁷ is independently a bond, alkylene, or heteroalkylene; and
M is alkylene or heteroalkylene.
104. The method of embodiment 103, wherein A⁴, A⁵, A⁶ and A⁷ are carboxyl.
105. The method of embodiment 103, wherein L⁴, L⁵, L⁶, and L⁷ are C₁-C₃ alkylene.
106. The method of embodiment 103, wherein L⁴, L⁵, L⁶, and L⁷ are C₁ alkylene.
107. The method of embodiment 103, wherein M is C₁-C₄ alkylene.
108. The method of embodiment 103, wherein M is C₂ alkylene.
109. The method of embodiment 103, wherein M is C₃ alkylene.
110. The method of embodiment 103, wherein M is C₁-C₆ heteroalkylene.
111. The method of embodiment 103, wherein M is C₆ heteroalkylene.
112. The method of embodiment 103, wherein M is bis(ethyleneoxy)ethylene.
113. The method of embodiment 103, wherein M includes side chains.
114. The method of embodiment 103, wherein M includes multiple side chains.
115. The method of embodiment 103, wherein M includes one or multiple carboxymethylene side chains.
116. The method of embodiment 103, wherein M includes one or multiple N-carboxymethylene groups or N-hydroxymethylene groups.
117. The method of embodiment 103, wherein the compound of Formula (II) includes three, four, five, six, or more N-carboxymethylene groups.
118. The method of embodiment 103, the compound of Formula (II) comprises ethylenediamine tetraacetic acid (EDTA).
119. The method of any of embodiments 103-118, wherein the compound of Formula (II) is a compound of Formula (II-a):

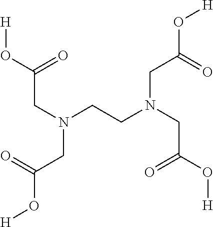 [SMILES: O=C(O)CN(CCN(CC(=O)O)CC(=O)O)CC(=O)O]

120. The method of any of embodiments 103-118, wherein the compound of Formula (II) is a compound of Formula (II-b):

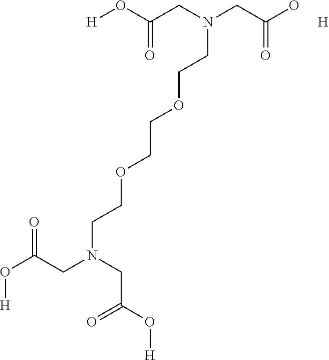

121. The method of any of embodiments 103-118, wherein the compound of Formula (II) is a compound of Formula (II-c):

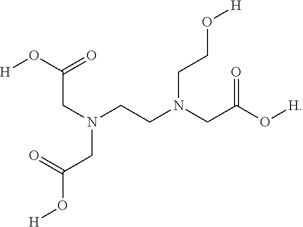

122. The method of any of embodiments 103-118, wherein the compound of Formula (II) is a compound of Formula (II-d):

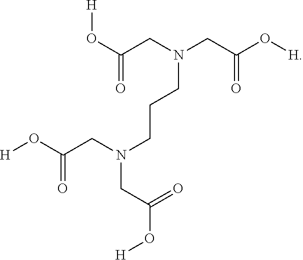

123. The method of any of embodiments 103-118, wherein the compound of Formula (II) is a compound of Formula (II-e):

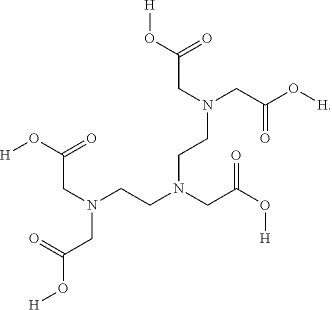

124. The method of any of embodiments 103-118, wherein the compound of Formula (II) is a compound of Formula (II-f):

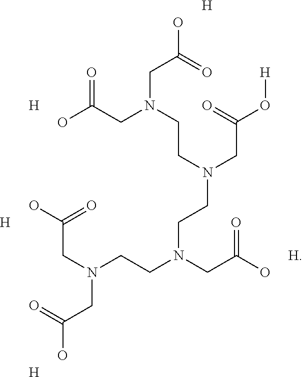

125. The method of embodiment 94, wherein the organic compound is a compound of Formula (III):

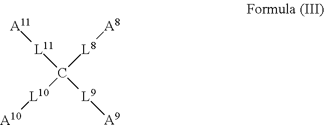

or a salt thereof, wherein:
   each of A⁸ and A⁹ is independently selected from an acidic group;
   each of A¹⁰ and A¹¹ is independently selected from an acidic group, a hydrogen atom, an alkyl, aryl, a hydroxy group, a thio group, and an amino group; and
   each of L⁸, L⁹, L¹⁰ and L¹¹ is independently a bond, alkylene, or heteroalkylene.
126. The method of embodiment 126, wherein A⁸, A⁹, and A¹⁰ are carboxyl.
127. The method of embodiment 126, wherein A¹⁰, A¹¹, are a hydrogen atom.
128. The method of embodiment 126, wherein A¹¹ is a hydroxy
129. The method of embodiment 126, wherein A¹¹ is or amino group.
130. The method of embodiment 126, wherein L⁸, L⁹, L¹⁰, and L¹¹ are a bond.
131. The method of embodiment 126, wherein L⁸ and L⁹ are C₁-C₃ alkylene.
132. The method of embodiment 126, wherein L¹¹ is a heteroalkylene.
133. The method of embodiment 126, wherein L¹¹ is methylenethiomethylene.
134. The method of embodiment 126, wherein the compound of Formula (III) comprises citric acid.
135. The method of embodiment 126, wherein the compound of Formula (III) comprises citric acid.
136. The method of embodiment 94, wherein the organic compound is a compound of Formula (IV):

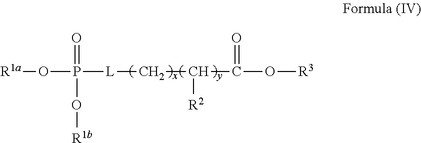

or a salt thereof, wherein:
   L is O, S, NH, or CH₂;
   each of R^{1a} and R^{1b} is independently H, an optionally substituted alkyl, or an optionally substituted aryl;
   R² is H, NR^{4a}R^{4b}, C(O)R⁵, or C(O)OR⁵;
   R³ is H, an optionally substituted alkyl, or an optionally substituted aryl;
   each of R^{4a} and R^{4b} is independently H, C(O)R⁶, or an optionally substituted alkyl;
   R⁵ is H, an optionally substituted alkyl, or an optionally substituted aryl;
   R⁶ is an optionally substituted alkyl or an optionally substituted aryl; and
   each of x and y is independently 0, 1, 2, or 3.
137. In some embodiments, the compound of Formula (III) is a compound of Formula (III-a):

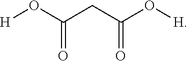

138. In some embodiments, the compound of Formula (III) is a compound of Formula (III-b):

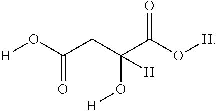

139. In some embodiments, the compound of Formula (III) is a compound of Formula (III-c):

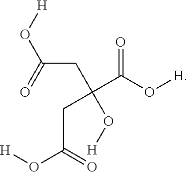

140. In some embodiments, the compound of Formula (III) is a compound of Formula (III-d):

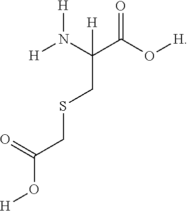

141. The method of embodiment 94, wherein the organic compound is a compound of Formula (IV):

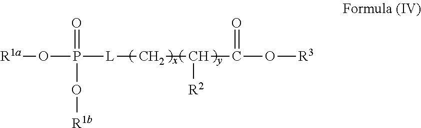

wherein L is O, S, NH, or CH₂, each of R^{1a} and R^{1b} is independently H, an optionally substituted alkyl, or an optionally substituted aryl, R² is H, NR^{4a}R^{4b}, C(O)R⁵, or C(O)OR⁵, R³ is H, an optionally substituted alkyl, or an optionally substituted aryl, each of R^{4a} and R^{4b} is independently H, C(O)R⁶, or an optionally substituted alkyl, R⁵ is H, an optionally substituted alkyl, or an optionally substituted aryl, R⁶ is an optionally substituted alkyl or an optionally substituted aryl, and each of x and y is independently 0, 1, 2, or 3.
142. The method of embodiment 141, wherein L is O or S. The method of embodiment 141, L is O.
143. The method of embodiment 141, wherein each of R^{1a} and R^{1b} is independently H.
144. The method of embodiment 141, wherein L is O, and each of R^{1a} and R^{1b} is H.
145. The method of embodiment 141, wherein R² is selected from H, NR^{4a}R^{4b}, and C(O)R⁵.
146. The method of embodiment 141, wherein R² is NR^{4a}R^{4b}.
147. The method of embodiment 141, wherein R² is NR^{4a}R^{4b} and each of R^{4a} and R^{4b} is independently H.
148. The method of embodiment 141, wherein L is O, each of R^{1a} and R^{1b} is independently H, R² is NR^{4a}R^{4b}, and each of R^{4a} and R^{4b} is independently H.
149. The method of embodiment 141, wherein R³ is H.
150. The method of embodiment 141, wherein L is O, each of R^{1a} and R^{1b} is independently H, R² is NR^{4a}R^{4b}, each of R^{4a} and R^{4b} is independently H, and R³ is H.
151. The method of embodiment 141, wherein each of x and y is independently 0 or 1.
152. The method of embodiment 141, wherein each of x and y is independently 1.
153. The method of embodiment 141, wherein L is O, each of R^{1a} and R^{1b} is independently H, R² is NR^{4a}R^{4b}, each of R^{4a} and R^{4b} is independently H, R³ is H, and each of x and y is 1.
154. The method of embodiment 141, wherein the compound of Formula (IV) is phosphoserine.
155. The method of embodiment 94, wherein the organic compound is a compound of Formula (V):

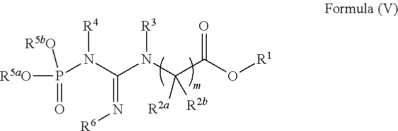

or a salt thereof, wherein:
R¹ is H, optionally substituted alkyl, optionally substituted alkenyl, optionally substituted alkynyl, optionally substituted aryl, or optionally substituted heteroaryl; each of R^{2a} and R^{2b} is independently H, optionally substituted alkyl, hydroxy, alkoxy, or halo;
each of R³ and R⁴ is independently H or optionally substituted alkyl;
each of R^{5a} and R^{5b} is independently H, optionally substituted alkyl, optionally substituted alkenyl, optionally substituted alkynyl, optionally substituted aryl, or optionally substituted heteroaryl;
R⁶ is H or optionally substituted alkyl; and
m is 1, 2, 3, 4, or 5.
156. The method of embodiment 155, wherein R¹ is H.
157. The method of embodiment 155, wherein each of R^{2a} and R^{2b} is independently H.
158. The method of embodiment 155, wherein m is 1.
159. The method of embodiment 155, wherein each of R³ and R⁴ is H.
160. The method of embodiment 155, wherein each of R^{5a} and R^{5b} is independently H.
161. The method of embodiment 155, wherein R⁶ is H.
162. The method of embodiment 155-161, wherein the compound of Formula (V) is a phosphocreatine.
163. The method of embodiment 94, wherein the compound is a compound of Formula (VI):

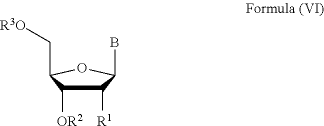

or a salt thereof, wherein:
B is a nucleobase;
R¹ is H, OR⁴, or halo;
R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl;
R³ is H, optionally substituted alkyl, or a phosphate moiety (e.g., monophosphate or diphosphate); and
R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl.
164. The method of embodiment 163, wherein B is a naturally occurring nucleobase or a non-naturally occurring nucleobase.
165. The method of embodiment 163, wherein B comprises adenine, cytosine, guanosine, thymine, or uracil.
166. The method of embodiment 163, wherein each of R¹, R², and R³ is H.
167. The method of embodiment 163, wherein R³ is a phosphate group.
168. The method of embodiment 167, wherein R³ is a monophosphate.
169. The method of embodiment 167, wherein R³ is a diphosphate.
170. The method of embodiment 167, wherein R³ is a triphosphate.
171. The method of any of embodiments 163-170, wherein the compound of Formula (VI) is Formula (VI-a):

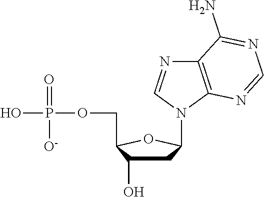

172. The method of any of embodiments 163-170, wherein the compound of Formula (VI) is Formula (VI-b):

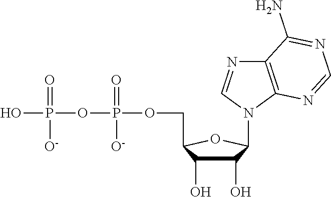

173. The method of any of embodiments 163-172, wherein the compound of Formula (VI) is 2′-deoxyadenosine monophosphate.
174. The method of any of embodiments 163-172, wherein the compound of Formula (VI) is 2′-deoxyadenosine diphosphate.
175. The method of any one of embodiments 74-174, wherein the organic compound is phosphoserine, calcium citrate, or malonic acid.
176. The method of any of embodiments 74-175, wherein the organic compound is phosphoserine.
177. The method of any of embodiments 33-176, wherein the composition has a viscosity between 100 cP and 10,000 cP.
178. The method of any of embodiments 33-177, wherein the composition further includes one or more additives.
179. The method of embodiment 178, wherein the one or more additives are selected from one or more of a filler, a formulation base, an abrasive (e.g., bone fragment), a coloring agent (e.g., dye, pigment, or opacifier), a flavoring agent (e.g., sweetener), a viscosity modifier (e.g., a polyol (e.g., glycerol, mannitol, sorbitol, trehalose, lactose, glucose, fructose, or sucrose)), a therapeutic, or a polymer.
180. The method of embodiment 179, wherein the one or more additives is a filler.
181. The method of embodiment 179, wherein the one or more additives is a formulation base.
182. The method of embodiment 179, wherein the one or more additives is an abrasive.
183. The method of embodiment 179, wherein the one or more additives is a coloring agent.
184. The method of embodiment 179, wherein the one or more additives is a flavoring agent.
185. The method of embodiment 179, wherein the one or more additives is a viscosity modifier.
186. The method of embodiment 179, wherein the one or more additives is a therapeutic.
187. The method of embodiment 179, wherein the one or more additives is a polymer.
188. The method of embodiment 187, wherein the polymer is one or more of poly(L-lactide), poly(D,L-lactide), polyglycolide, poly(O-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin and mixtures thereof.
189. The method of embodiment 188, wherein the polymer is poly(L-lactide).
190. The method of embodiment 188, wherein the polymer is poly(D,L-lactide).
191. The method of embodiment 188, wherein the polymer is polyglycolide.
192. The method of embodiment 188, wherein the polymer is poly(O-caprolactone).
193. The method of embodiment 188, wherein the polymer is poly(teramethylglycolic-acid).
194. The method of embodiment 188, wherein the polymer is poly(dioxanone).
195. The method of embodiment 188, wherein the polymer is poly(hydroxybutyrate).
196. The method of embodiment 188, wherein the polymer is poly(hydroxyvalerate).
197. The method of embodiment 188, wherein the polymer is poly(lactide-co-glycolide).
198. The method of embodiment 188, wherein the polymer is poly(glycolide-co-trimethylene carbonate).
199. The method of embodiment 188, wherein the polymer is poly(glycolide-co-caprolactone).
200. The method of embodiment 188, wherein the polymer is poly(glycolide-co-dioxanone-co-trimethylene-carbonate).
201. The method of embodiment 188, wherein the polymer is poly(tetramethylglycolic-acid-co-dioxanone-co-trimethylenecarbonate).
202. The method of embodiment 188, wherein the polymer is poly(glycolide-co-caprolactone-co-lactide-co-trimethylene-carbonate).
203. The method of embodiment 188, wherein the polymer is poly(hydroxybutyrate-co-hydroxyvalerate).
204. The method of embodiment 188, wherein the polymer is poly(methylmethacrylate).
205. The method of embodiment 188, wherein the polymer is poly(acrylate).
206. The method of embodiment 188, wherein the polymer is a polyamine.
207. The method of embodiment 188, wherein the polymer is a polyamide.
208. The method of embodiment 188, wherein the polymer is a polyimidazole.
209. The method of embodiment 188, wherein the polymer is poly(vinyl-pyrrolidone).
210. The method of embodiment 188, wherein the polymer is collagen
211. The method of embodiment 188, wherein the polymer is silk.
212. The method of embodiment 188, wherein the polymer is chitosan.
213. The method of embodiment 188, wherein the polymer is hyaluronic acid.
214. The method of embodiment 188, wherein the polymer is gelatin.
215. The method of any of embodiments 178-214, wherein the one or more additives are provided as a powder.
216. The method of embodiment 215, wherein a particle size of the powder is about 0.001 mm to about 1.000 mm.
217. The method of any of embodiments 178-214, wherein the one or more additives are provided as granules.
218. The method of any of embodiments 178-214, wherein the one or more additives are provided as solutes.
219. The method of any of embodiments 178-214, wherein the one or more additives are provided as fibers.
220. The method of embodiment 219, wherein a diameter of the fibers are 0.010 mm to about 2 mm.
221. The method of any of embodiments 33-220, wherein the composition further comprises an agent.
222. The method of embodiment 221, wherein the agent is a contrast agent.
223. The method of embodiment 222, wherein the contrast agent comprises barium.
224. The method of embodiment 222, wherein the contrast agent comprises iodine.
225. The method of embodiment 222, wherein the contrast agent comprises bismuth.
226. The method of embodiment 222, wherein the contrast agent comprises gadolinium.
227. The method of embodiment 222, wherein the contrast agent comprises CO₂.
228. The method of embodiment 222, wherein the contrast agent comprises hydrogen.
229. The method of embodiment 222, wherein the contrast agent comprises air.
230. The method of any one of embodiments 221-229, wherein the agent is a radioisotope.
231. The of any of embodiments 221-230, wherein an amount of the agent in the composition is less than about 20%, 15%, 10%, 9%, 8%, 7%, 6%, 5%, 4%, 3%, 2%, 1%, 0.5%, 0.1%, 0.05%, or 0.01% (w/w or w/v) of the total composition.
232. The of any of embodiments 33-231, wherein the composition has a tacky state for up to 12 minutes.
233. The method of embodiment 232, the tacky state has a tack stress of about 10 kPa to about 250 kPa.
234. The of any of embodiments 33-233, wherein the composition has an adhesive strength upon curing of about 100 kPa to about 12,000 kPa.
235. The of any of embodiments 33-234, wherein the composition has a viscosity of about 100 cP to about 10,000 cP when in a fluid state.
236. The of any of embodiments 33-235, wherein the composition has a viscosity of about 10,000 cP to about 250,000 cP when in a semi-solid or tacky state.
237. The method of embodiments 235 or 236, wherein the viscosity of the composition is such that the composition does not substantially extravasate into surrounding tissues during application.
238. The method of any of embodiments 74-237, wherein the amount of the multivalent metal compound is about 10% to about 90% weight by weight (w/w) of the composition.
239. The method of any of embodiments 33-238, wherein the composition is formulated as an injectable composition.
240. A kit for positioning two or more segments or structures, including:
   one or more devices or device components for positioning the two or more bone segments or structures; and
   a composition to be applied to the two or more segments or structures.
241. The kit of embodiment 240, wherein the composition is an adhesive composition.
242. The kit of embodiment 240, wherein the composition is a bone restorative composition.
243. The kit of embodiment 50, wherein the composition includes solid components including a multivalent metal salt and an organic compound and liquid components including an aqueous medium.
244. The kit of embodiment 51, wherein the solid components and the liquid components of the adhesive composition are provided in separate packaging.
245. The kit of any of embodiments 240-244, wherein the composition is the composition of any of embodiments 74-239.
246. The kit of any of embodiments 240-245, further including a device for application of the adhesive composition.
247. The kit of embodiment 246, wherein the device for application of an adhesive composition is a syringe.
248. A device for reproducibly positioning, relating and fixating two or more bone segments or other structures to one another in order to repeatably establish a mechanically stable spatial relationship allowing for definitive fixation, wherein the device comprises:
   one or more retaining elements, base elements, stops, connectors, tines, arms and contact points, wherein the device is used for:
      (i) prepositioning the bone segments or other structures in a desired position and relationship;
      (ii) reversibly distracting the segments or structures to create a gap between the bone segments or structures;
      (iii) adhering the two or more bone segments, or other structures with a composition;
      (iv) repositioning the bone segments, bone fragments, or hardware devices to the previously established spatial relation; and
      (v) stably maintaining that spatial relationship during the course of curing of the composition.
249. The device of embodiment 248, wherein the device further comprises a rail element for movement of the related bone segment or structure.
250. The device of any one of embodiments 248-249, wherein the device further comprises a hinge element for movement of the related bone segment or structure.
251. The device of any one of embodiments 248-250, wherein each retaining element grasps one bone segment or structure.
252. The device of any one of embodiments 248-251, wherein each retaining element grasps part of a bone segment or structure.
253. The device of any one of embodiments 248-252, wherein the retaining elements comprise a plurality of opposing sharp points or edges for grasping and holding a bone segment or structure, which are capable of penetrating soft tissue and engaging bone.
254. The device of any one of embodiments 248-253, wherein the device is capable of being selectively locked to limit movement of the bone segment or structure being held by the retaining element.
255. The device of any one of embodiments 248-254, wherein the stable spatial relationship may be irreversibly and selectively locked by a structural feature of the device of embodiment 1 for one or more of the six degrees of freedom of relative motion of the one or more bone segments, bone fragments, or hardware devices.
256. The device of any one of embodiments 248-255, wherein a stable spatial relationship may be substantially maintained while one or more of the six degrees of freedom of motion, i.e., three rotational and three translational, in isolation, allows application of force, e.g., compression of chosen magnitude, across the gap pairwise separating the segments, bone fragments, or hardware devices.
257. The device of any one of embodiments 248-256, wherein the size, the shape, the mechanical parameters, e.g., stiffness, range of motion of the movable elements, e.g., angular or translational, and the magnitude of forces that may be applied by the grasping elements of the device to the one or more bone segments, bone fragments, or hardware devices during the course of action of the device of embodiment 1 may be selected to the fit the clinical context, i.e., the size of the bone segments, bone fragments, or hardware devices, their degree of fragility, or the ease of access to the site of fixation.
258. The device of any one of embodiments 248-257, wherein the device provides a means for fixation without use of or introduction of a permanent screw or plate to the one or more bone segments or bone fragments.
259. A device for positioning or fixating one or more bone segments or bone fragments, the device comprising:
   a rail disposed substantially parallel to the one or more bone segments or bone fragments to be positioned or fixated;
   a plurality of base elements slidably coupled to and disposed along a length of the rail; and
   a plurality of retaining elements, each having a first end operatively coupled to the plurality of base elements and a second end constructed and arranged to hold at least one of the one or more bone segments or bone fragments.
260. The device of embodiment 259, wherein the rail is rectilinear, curved, or comprises offsets, the departure from linearity being in any one or several dimensions, e.g., following a circular arc, a spiral, and curving approximately in the plane of the retaining elements or anu other dimension
261. A device for positioning or fixating one or more bone segments or bone fragments, the device comprising:
   a base comprising one or more arms connected by a hinge;
   one or more retaining elements, each having a first end slidably coupled along a length of one of the one or more arms and a second end constructed and arranged to hold at least one of the one or more bone segments or bone fragments.
262. A device for positioning or fixating one or more bone segments or bone fragments, the device comprising one or more of the following to allow for movement between its elements:
   a hinge, a ball-and-socket joint, a double ball-and-socket joint, or a rail with sliding bases.
263. The device of embodiment 261, wherein the connection between each of the plurality of base elements and the first ends of the plurality of retaining elements permits up to three degrees of rotational freedom and up to three degrees of translational freedom of the each of the plurality of retaining elements relative to the rail.
264. The device of embodiment 263, wherein the connection between each of the plurality of base elements and the first ends of the plurality of retaining elements is securable following adjustment along one or more rotational and/or translation degrees of freedom.
265. The device of embodiment 263, wherein the rigidity is provided mechanically or chemically.
266. The device of any of embodiments 263-264, wherein the connection between each of the plurality of base elements and the first ends of the plurality of retaining elements comprises a hinge, a telescopic member, or ball-and-socket connection.
267. The device of embodiment 261, wherein a position of each of the plurality of base elements along the rail is secured mechanically.
268. The device of embodiment 261, wherein each of the plurality of base elements further comprises a protrusion adapted to connect to an external support.
269. The device of embodiment 261, wherein the rail further comprises one or more stops along its length arranged to limit translation of the plurality of base elements.
270. The device of any one of embodiments 259-269, wherein each of the plurality of retaining elements comprises one or more arms.
271. The device of any one of embodiments 259-269, wherein each of the plurality of retaining elements comprises a surface capable of supporting bone segments.
272. The device of embodiment 271, wherein the surface comprises a cylindrical concave surface, conical concave surface, a flat surface, or a convex surface.
273. The device of any one of embodiments 259-272, wherein each of the plurality of retaining elements comprises one or more contact points capable of supporting one or more bone fragments or bone segments.
274. The device of embodiment 273, wherein each of the one or more contact points comprise one or more tines.
275. The device of embodiment 273 or 274, wherein the one or more contact points are disposed orthogonal to the one or more arms of the plurality of retaining elements.
276. The device of any one of embodiments 259-275, further comprising a bracing element constructed and arranged to connect the device to an external support.
277. The device of any one of embodiments 259-276, wherein the one or more bone fragments or bone segments to be positioned or fixated further comprise one or more of a synthetic implantable object, a biologically generated implantable object, or a biologically generated tissue or material.
278. The device of any one of embodiments 259-278, constructed and arranged to position the one or more bone fragments or bone segments in contact with an adhesive composition.
279. A method of fixating one or more bone segments or bone fragments at a location, the method comprising:
   attaching the one or more bone segments or bone fragments to one or more retaining elements of a fixation device; and
   positioning the one or more bone segments or bone fragments in a position; and
   securing the position of the one or more bone segments or bone fragments.
280. The method of embodiment 279, wherein positioning the one or more bone segments or bone fragments comprises positioning one or more base elements coupled to the one or more retaining elements along a rail.
281. The method of embodiment 280, further comprising fixing the position of the one or more retaining elements mechanically or chemically.
282. The method of embodiment 281, wherein fixing the position of the one or more retaining elements mechanically comprises applying a compressive force to the one or more retaining elements.
283. The method of embodiment 282, wherein positioning the one or more structures comprises actuating a hinge operatively coupled to the one or more retaining elements.
284. The method of any one of embodiments 279-283, wherein positing the positioning the one or more bone segments or bone fragments comprises positioning the one or more bone segments or bone fragments such that gaps are present between the one or more bone segments or bone fragments to be positioned.
285. The method of embodiment 284, further comprising applying an adhesive composition between the gaps of the positioned one or more bone segments or bone fragments.
286. The method of embodiment 285, further comprising applying a compressive force to the adhesive covered surfaces of the one or more bone segments or bone fragments.
287. The method of embodiment 284 or 285, further comprising curing the adhesive composition, thereby fixating the one or more bone segments or bone fragments.
288. The method of any one of embodiments 279-287, wherein the fixation device is the fixation device of any one of embodiments 248-278.
289. A kit, comprising:
   a fixation device for fixating a position of one or more bone structures or bone fragments; and
   an adhesive composition comprising a multivalent metal salt, an organic compound, and an aqueous medium.
290. A kit, comprising:
   one or more retaining elements connectable to a fixation device for fixating a position of one or more bone structures or bone fragments; and
   an adhesive composition comprising a multivalent metal salt, an organic compound, and an aqueous medium.
291. The kit of embodiment 289 or 290, wherein the fixation device is the fixation device of any one of embodiments 248-278.
292. The kit of any of embodiments 289-291, further comprising a device for application of the adhesive composition.
293. The kit of any of embodiments 289-292, further comprising a device for preparation of the adhesive composition.
294. The kit of any of embodiments 289-293, wherein the adhesive composition comprises a multivalent metal salt, an organic compound, and an aqueous medium.
295. The kit of embodiment 294, wherein the multivalent metal salt comprises calcium.
296. The kit of embodiment 295, wherein said multivalent metal salt is selected from the group consisting of calcium phosphate, tricalcium phosphate, tetracalcium phosphate, and mixtures thereof.
297. The kit of embodiment 296, wherein said multivalent metal salt comprises tetracalcium phosphate.
298. The kit of any of embodiments 289-297, wherein said organic compound is selected from the group consisting of phosphoserine, carboxy ethyl phosphonate, phosphonoacetic acid, phosphocreatine, and mixtures thereof.
299. The kit of embodiment 298, wherein said organic compound comprises phosphoserine.
300. The kit of any of embodiments 289-299, wherein said aqueous medium is selected from the group consisting of water or a blood-based product.
301. The kit of embodiment 300, wherein the aqueous medium comprises sodium hydroxide.
302. The kit of any of embodiments 42-54, wherein the adhesive composition, when mixed, has a separation strength of about 10 kPa to about 150 kPa after mixing.
303. The kit of any of embodiments 289-302, wherein the adhesive composition, when mixed, has a working time of about 30 seconds to about 12 minutes after mixing.
304. The kit of any of embodiments 289-302, wherein the adhesive composition, when mixed, has a working time of about 15 seconds to about 5 minutes after mixing.
305. The kit of any of embodiments 289-304, wherein the adhesive composition has an adhesive strength upon curing about 250 kPa to about 1000 kPa.
306. The kit of any of embodiments 289-305, wherein said multivalent metal salt and said organic compound are each independently present in the adhesive composition in an amount from about 10% to about 90% (e.g., 10% to 75%, 25 to 50%) w/w, w/v, or v/v of the total weight of all components in the adhesive composition.
307. The kit of any of embodiments 289-306, wherein said aqueous medium is present in an amount up to about 10% or more, e.g., 15%, 20%, 25%, 30%, or 35%, w/w, w/v, or v/v of the total weight of all components in the adhesive composition.
308. A device for positioning or fixating one or more structures, the device comprising:
   a rail disposed substantially parallel to the one or more structures to be positioned or fixated;
   a plurality of base elements slidably coupled to and disposed along a length of the rail; and
   a plurality of retaining elements, each having a first end operatively coupled to the plurality of base elements and a second end constructed and arranged to hold at least one of the one or more segments or structures.
309. The device of embodiment 308, wherein the device is constructed and arranged to bond or attach to the one or more segments or structures to be fixated in one or more locations using a mechanical connection without the mechanical feature fitting within a kerf space between the one or more segments or structures.
310. A device for positioning or fixating one or more structures, the device comprising:
   a first linkage arm including a hinge and one or more retaining elements; and
   a second linkage arm including at least one ball joint and one or more retaining elements, the first and second linkage arms constructed and arranged to be independently positioned to hold at least one of the one or more segments or structures.
311. The device of any one of the preceding embodiments wherein the retaining elements are shaped to allow a surgeon better access to a surgical site.
312. The device of any embodiment disclosed herein, wherein the retaining elements can be shaped or elongated to allow a surgeon better access to an operating field.
313. The device of any embodiment disclosed herein, further comprising a handles operatively coupled to the plurality of retaining elements of constructed and arranged to be detached to allow a surgeon better access to an operating field, to reduce the bulk of the device, or to leave the remaining device on the bone fulfilling its duty to hold the one or more segments or structures in position.

### EXAMPLES

The Examples set forth below are intended to aid in the understanding of the disclosure, but are not intended to, and should not be construed to, limit its scope in any way.

### Example 1: Exemplary Adhesive Compositions for Use with a Device

Table 1 lists exemplary compositions for use as a bone adhesive to fill in the kerf space or gap defects in bone and fixate bone segments. These adhesive compositions may be used with any of the devices as described in this application (e.g., an HFD or RFD). While water was used as the aqueous medium in these exemplary compositions, the aqueous medium may instead be saline, blood, saliva, serum, or a blood-based solution or suspensions. The solid components (i.e., calcium compounds, e.g., tetracalcium phosphate) listed in the Table 1 were supplied as particles, however, they may be supplied in granule, or fiber form. In some embodiments, the resulting properties such as working time, setting time, tack strength, and adhesive strength would be affected by these changes. The specific mean particle, granule, or fiber size for each solid component can be selected to satisfy the use requirements. The quantities of each of the components listed may be altered or adjusted in relation to the other components in the composition.

TABLE 1
Exemplary Adhesive Compositions

| | | | | Tetra- | |
|---|---|---|---|---|---|
| | | | | calcium | |
| Compo- | Compound | Compound | Compound | Phosphate | Water |
| sition | Name | Formula | (mg) | (mg) | (μl) |
| 1A | Glyphosine | I-a | 711 | 800 | 540 |
| 1B | ATMP | I-b | 202 | 800 | 270 |
| 1C | EDTA¹ | II-a | 395 | 800 | 270 |
| 1D | EDTA² | II-a | 790 | 800 | 370 |
| 1E | EDTA³ | II-a | 550 | 800 | 320 |
| 1F | PDTA | II-c | 414 | 800 | 270 |
| 1G | Malonic | III-a | 281 | 800 | 135 |
| | Acid | | | | |
| 1H | Citric | III-c | 519 | 800 | 260 |
| | Acid | | | | |
| 1I | OPLS | IV-a | 500 | 800 | 270 |

### Example 2. Adhesive Shear Strength to Bone Substrate Surfaces

The shear strength that it took to rupture the bond formed between two bone block surfaces using many of the adhesive compositions listed in Table 1 was measured to determine which exemplary adhesive composition provided the greatest strength. Each composition was prepared by mixing for 20 seconds the dry components, i.e., Compound and tetra-calcium phosphate as can be found in Table 1, after addition of the water to ensure a smooth consistency in a 25 mL capacity silicone mixing bowl using a stainless-steel spatula. After mixing, the composition was loaded into a 3 cc capacity slip tip syringe and immediately injected onto one end of each of the 8.5 mm×8.5 mm surfaces of two rectangular bovine bone blocks that were each 10-15 mm long. Immediately thereafter, the bone block surfaces covered with adhesive composition were opposed and pressed together and excess material that squeezed out which surrounded the perimeter of the external surfaces of the joint was removed with a spatula. The adjoined blocks were placed into a fixture that applied a slight compressive force of 3 to 5 N for 4 minutes from the start of mixing corresponding to the working period of the compositions from the start of mixing. Thereafter, the blocks were removed from the fixture and submerged into a phosphate buffered saline (PBS) solution bath at 37° C. to allow the compositions to cure for 24 hours from the start of mixing. After curing, the blocks were removed from the PBS bath for shear testing. The proximal block of the adhered block set was secured in a stable fashion to prevent movement within a sample holding fixture up to within 1.0 mm of the adhered joint mounted to an INSTRON® 5969 axial load frame. The distal block of the adhered block set was cantilevered from the sample holding fixture. The INSTRON® crosshead with an attached anvil fixture was lowered until the distal surface of the anvil was within 0.5 mm of the top surface of the distal bone block and within 1.0 mm of adhered joint. The test was run with the crosshead speed at 2 mm/minute. Table 2 shows the results for the average shear stress (MPa) and standard deviation (MPa) after 24 hours of cure in order to rupture the bond formed at the joint between the adhered bone blocks.

TABLE 2
Adhesive Shear Strength to Rupture Bond of Bone
Blocks Adhered Together after Exemplary Adhesive
Compositions Cured for 24 Hours in PBS Solution

| | | Average Shear | Standard |
|---|---|---|---|
| | | Stress (MPa), | Deviation |
| Composition | Compound name | n = 3 | (MPa) |
| 1A | Glyphosine | 0.37 | 0.09 |
| 1B | ATMP | 0.35 | 0.18 |
| 1C | EDTA¹ | 1.76 | 0.58 |
| 1D | EDTA² | 1.30 | 0.73 |
| 1E | EDTA³ | 2.23 | 0.76 |
| 1F | Propylenediamine- | 0.18 | 0.02 |
| | tertaacetic Acid | | |
| 1G | Malonic Acid | 1.68 | 0.54 |
| 1H | Citric Acid | 3.16* | n/a |
| 1I | OPLS | 2.36 | 0.23 |

*n = 2 samples tested

### Example 3: Long Bone Fracture Repair Utilizing RFDs

In an exemplary procedure, multiple rail fixation devices RFDs were utilized to aid in the fixation and repair of a fractured long bones. FIGS. 3A-3D illustrate the repair of a humerus using a device with a plurality of retaining elements REs and FIGS. 4A-4E illustrate the repair of a radius using a device with a plurality of REs.

First, each portion of fractured bone was grasped via the retaining elements RE of each device, with one device retaining each bone segment. Then, with the connector elements C of each device in an unlocked state, the RE bases were fixated to the common rail R, with R approximately parallel to the bone being fixated. R was placed in the surgical field in a position so as to not interfere with application of the adhesive composition or movement of the bone segments. Once each bone segment was retained by a device, the segments were translated away from each other by moving the RE bases along the rail R. This method of translation ensured the bone segments were only capable of moving along the translational axis and back into their appropriate spatial relationship. Once the segments were distanced from each other, connector components C were locked via ball-and-socket joints. Next, an adhesive composition was applied to the inner sides of the segments. The chosen adhesive composition used herein was that listed as Composition 11 in Table 1, above. Once the adhesive composition was fully applied, the bone segments were translated back to their appropriate positions via translation of RE bases. A slight compressive force, e.g., 3-5N, was applied between the bone segments via an elastic band which engaged the retaining elements RE of all devices engaged in the procedure. After approximately 10 minutes, the time needed for the adhesive composition to cure, the elastic band was removed and the RE attached to each bone segment was detached. The adhesive composition provided the bone segments sufficient strength to withstand fixation and the strength will grow over time. Once all the devices were removed, the surgeon closed the surgical site via normal surgical means. There was no need for a secondary procedure.

### Example 4: Mandible Fracture Fixation in a Canine Cadaver

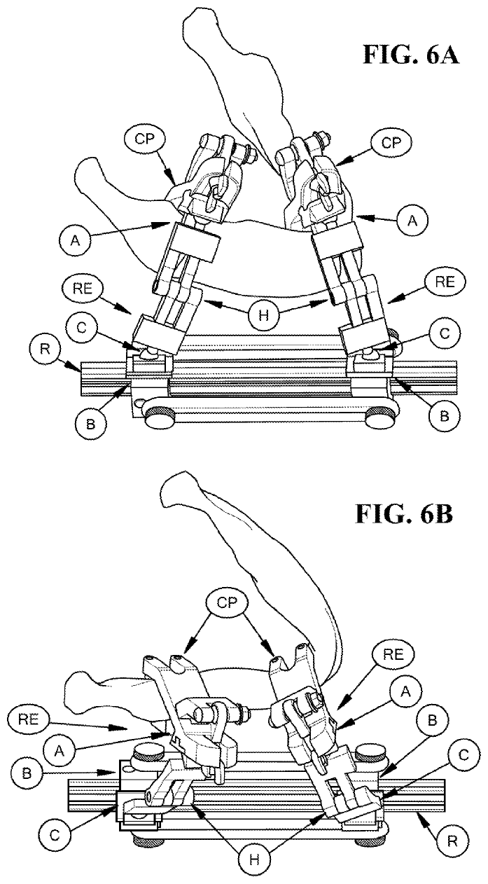

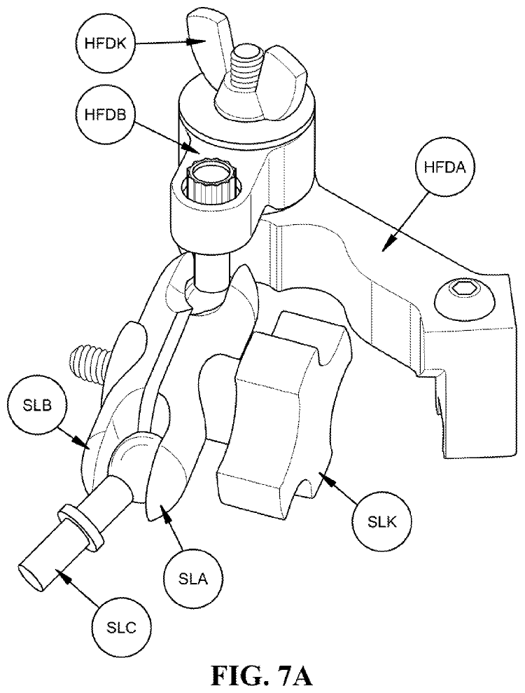

An exemplary HFD, illustrated in FIGS. 5A-5C, is to be used in a surgical procedure to temporarily fixate bone segments of a canine mandible. The HFD was chosen for this procedure because it was a small bone fracture and only two bone segments needed to be manipulated. In the first step, a surgeon will cut into the surgical area, clean the area, and reduce the bone segments as needed, leaving separated bone segments and kerf space between. Once these steps are completed, an HFD will be placed perpendicular to each bone segment, and said bone segments grasped by the HFD. The device will include two binder clip-like clamps which will be crimped onto the proximal and distal segments of each bone segment. A rubber band will be wound around the finger grips of the clamps to reduce (compress) the two segments of bone together. Thereafter, an adhesive composition to be applied to the fracture site will be mixed. The chosen adhesive composition used will be Composition 1I in Table 1. After approximately 45 seconds from the start of mixing, the adhesive composition will be loaded into a 3 cc syringe. Each HFD will move its attached bone segment away from its anatomical position such that the adhesive composition can be delivered onto the interior surface of each segment. This action will be performed by the base element B of each HFD being translated apart from each other along a common rail element R, this configuration is illustrated in FIGS. 6A-6B. Following separation of each bone segment and delivery of the adhesive composition, the fracture will be re-reduced to its proper anatomical position and allowed to cure under compression provided by the rubber band wound around the clamps of each HFD. After approximately 10 minutes from the start of mixing the adhesive composition, each HFD will be removed and the strength of the bond qualitatively tested through manual bending.

### Example 5: Hinge Fixation Device with Multi-Axis Positioning

An embodiment of a fixation device having two separate devices, each with different degrees of freedom, is illustrated in FIGS. 7A and 7B. In FIGS. 7A and 7B, the fixation device includes a rotary hinge-type 1-DOF linkage (HFD) and a twin ball joint type 6-DOF linkage (SL). The 1-DOF linkage is used to reduce or distend a fracture. With the 1-DOF linkage, the compressive force under reduction can be controlled. As illustrated, the 1-DOF linkage includes two arms (HFDA and HFDB) articulating on a hinge. The hinge can be locked using a twisted knob (HFDK) under various levels of compressive forces and under distention. The hinge may also be equipped with a spring to adjust the force of compression/distention. The 6-DOF linkage is used to manipulate the alignment between one or more fractured bone fragments. Any particular alignment of the one or more fractured bone fragments is selectively locked into position. As illustrated in FIGS. 7A-7B, the 6-DOF linkage includes two plates (SLA and SLB) and two balls (SLC). The balls can be locked to fix the alignment using a twisted knob (SLK). The 1-DOF and 6-DOF linkages, in series, bridge two retaining elements (RE). As illustrated, the RE are a four-pronged forceps that are used to grasp the one or more fractured bone fragments.

### Example 6: Hinge Fixation Device with Multi-Axis Positioning

An exemplary method of fixing the position of one or more bone fragments using the fixation device illustrated in FIGS. 7A and 7B is illustrated in FIGS. 8A-8R. As illustrated in FIG. 8A, the fixation device of FIGS. 7A and 7B was used to temporarily fixate bone segments of a canine mandible, but may be used for bones of any size and shape. FIGS. 8B and 8C illustrate the attachment of the bone segments of the canine mandible to each of the 1-DOF linkage (FIG. 8B) and 6-DOF linkage (FIG. 8C) of the fixation device. FIGS. 8D-8F illustrate using the fixation device to manipulate the bone segments of a canine mandible in various positions. FIGS. 8G and 8H illustrate the arrival at (FIG. 8G) and locking into place of the correct position (FIG. 8H) of the bone segments of the canine mandible by locking the 6-DOF linkage. FIG. 8I illustrates the marking and drilling of holes into the bone segments of the canine mandible for accepting a permanent fixation mechanism to be attached, such as a bone plate. FIGS. 8J-8L illustrate the manipulation of the 1-DOF linkage to adjust the compressive reduction and distention of the fracture between the bone segments of the canine mandible.

As further illustrated in FIGS. 8J-8L is the attachment of one end of a bone plate to one of the bone segments of the canine mandible with the other end free until the bone segments of the canine mandible are repositioned. FIG. 8M illustrates the application of an adhesive, e.g., as disclosed herein, to the ends of the bone segments of the canine mandible with the distended 1-DOF linkage providing sufficient space between the bone segments of the canine mandible for a suitable application. While the use of an adhesive is illustrated, it is to be appreciated that the fixation device of this example can be used to align and position one or more bone fragments for definitive fixation, e.g., fixation using hardware such as plates, screws, and wiring, without requiring the application of an adhesive. FIGS. 8N and 8O illustrate the repositioning of the bone segments of the canine mandible, e.g., compressive reduction of the fracture between the bone segments of the canine mandible, to bring the adhesive-coated ends of the bone segments of the canine mandible into contact. When the adhesive-coated ends of the bone segments of the canine mandible are in correct alignment, the 1-DOF linkage is locked (FIG. 8O) to secure this position. FIG. 8P illustrates the removal of excess adhesive from the position-locked bone segments of the canine mandible. FIG. 8Q illustrates the securement of the bone plate, e.g., using screws, to the bone segments of the canine mandible. FIG. 8R illustrates the stabilization of the fractured bone segments of the canine mandible until the applied adhesive has cured.

### Example 7: Fixation Device with Retaining Elements Having Tined Jaws

Exemplary retaining elements including tined jaws for a fixation device are illustrated in FIGS. 9A-9B. As illustrated in FIG. 9A, a retaining element RE can have three contact points that render the RE suitable for use in a limited access space, such as a space that is less than 20 mm in dimension. One of the REs in FIG. 9A is a two-tined jaw TTJ and provides two contact points. The other RE in FIG. 9A is an opposing one-tined jaw OTJ that provides one contact point against the structure, e.g., bone, being fixated.

FIG. 9B illustrates two REs, with the top RE being a TTJ as described above. The lower RE is a variant of a TTJ that includes a pivot point or hinge H in the center, this RE is called a hinged tined jaw HTJ. The hinge H permits motion of the HTJ in the plane of the structure, e.g., bone, being fixated and can provide an improved hold against the structure should it have an irregular surface.

### Example 8: Fixation Device with Handles

Exemplary retaining elements that are elongated are illustrated in FIGS. 10A-10C. FIG. 10A depicts a positioning device with elongated retaining elements RE1 and RE2 which allows expanded surgical access at the fracture or defect location, RE1 and RE2 include an articulating locking mechanism L which allows up to three degrees of freedom for aligning and positioning bone fragments and one degree of freedom for fracture or defect distention and compression. Both RE1 and RE2 terminate in handles HA1, HA2 that allow for manipulation of the grasping end of RE1 and RE2. In addition, handles HA1, HA2 are detachable from the retaining elements RE1 and RE2 to allow better access to the surgical site and to reduce the bulk of the positioning device. In operation, the handles HA1, HA2 can be translated along their long axis to actuate the retaining elements RE1 and RE2 to grasp the structures, i.e., bones, to be fixated and hold them in position, as shown in by the arrows in FIG. 10B.

FIG. 10C depicts the rail fixation device RFD shown in FIG. 10A with detachable handles HA1 and HA2 on the retaining elements RE1 and RE2. The handles HA1 and HA2 are detachable at the articulating locking mechanism L. As discussed above, having handles HA1 and HA2 be disposed away from the RE1 and RE2 terminate provide for the ability to manipulate RE1 and RE2 from an external position should the space around the segments, i.e., bones, to be fixated be narrow or otherwise too small for an end user to manipulate the RE1 and RE2 using their hands or a hand tool.

1. A device for positioning two or more segments or structures, the device comprising:
one or more base elements;
a plurality of retaining elements, each retaining element having a first end operatively coupled to one of the one or more base elements and a second end constructed and arranged to hold at least one of the segments or structures; and
one or both of a rail component or hinge component for providing positioning to the two or more segments or structures.

2. The device of claim 1, wherein the rail component is rectilinear.

3. The device of claim 1, wherein the rail component is curved or comprises offsets.

4. The device of claim 1, wherein the rail component comprises one or more portions to achieve a combination of rectilinear and curved segments.

5. The device of claim 1, wherein the plurality of retaining elements are attached to the rail element via the one or more base elements.

6. The device of claim 1, wherein the one or more base elements comprise a hinge which terminates in one or more arms for holding or grasping the bone segments or structures to be fixated.

7. The device of claim 1, wherein the one or more retaining elements comprise a first end slidably coupled along a length of one of the one or more arms and a second end constructed and arranged to hold one of or part of one of the two or more segments or structures to be fixated.

8. The device of claim 1, wherein each retaining element comprises a plurality of opposing sharp points or edges constructed and arranged to penetrate soft tissue and engage bone.

9. The device of claim 1, wherein each retaining element comprises a surface constructed and arranged to support the two or more segments or structures.

10. The device of claim 9, wherein the surface comprises a cylindrical concave surface, conical concave surface, a flat surface, or a convex surface.

11. The device of claim 1, wherein the device further comprises one or more of a hinge, a ball-and-socket joint, a double ball-and-socket joint, a telescopic member, or a rail with sliding bases.

12. The device of claim 1, wherein the connection between each of the plurality of base elements and the plurality of retaining elements is constructed and arranged to be locked to a position.

13. The device of claim 1, wherein each of the plurality of base elements further comprises a protrusion adapted to connect to an external support.

14. The device of claim 1, wherein the rail component further comprises one or more stops along its length constructed and arranged to limit translation of the plurality of base elements.

15. The device of any one of claims 1-7, wherein each of the plurality of retaining elements comprises one or more contact points for contacting or grasping the two or more segments or structures.

16. The device of claim 15, wherein each of the one or more contact points comprises one or more tines.

17. The device of claim 15, wherein the one or more contact points are disposed orthogonal to one or more arms of the plurality of retaining elements.

18. The device of claim 1, further comprising handles having a length that are constructed and arranged to be connectable to each of the plurality of retaining elements.

19. The device of claim 18, wherein the handles are detachable from the device.

20. The device of claim 1, wherein the device further comprises:
a first linkage arm comprising a hinge and one or more retaining elements; and
a second linkage arm comprising at least one ball joint and one or more retaining elements, wherein the first and second linkage arms are constructed and arranged to be independently positioned to hold at least one of the segments or structures to be fixated.

21. A method of positioning two or more segments or structures, comprising:
prepositioning the two or more segments or structures in an initial position;
attaching at least one positioning device to each of the two or more segments or structures;
distracting the two or more segments or structures such that a gap is created between each of the two or more segments or structures;
applying a composition to the gap between the two or more segments or structures; and
repositioning the two or more segments or structures with the applied composition to the initial position, thereby positioning the two or more segments or structures as the composition cures.

22. The method of claim 21, wherein the two or more segments or structures to be positioned comprise bone segments, bone fragments, or other biologically compatible structures.

23. The method of claim 21, wherein the positioning device comprises:
one or more base elements;
a plurality of retaining elements, each retaining element having a first end operatively coupled to one of the one or more base elements and a second end constructed and arranged to hold at least one of the two or more segments or structures; and
one or both of a rail component or hinge component for providing positioning to the two or more segments or structures.

24. The method of claim 23, wherein the rail component of the positioning device is disposed substantially parallel to the two or more segments or structures.

25. The method of use of claim 23, wherein the hinge component is attached to a base element of the device and comprises one or more arms for grasping the two or more segments or structures.

26. The method of claim 23, wherein the one or more base elements are slidably coupled to and disposed along a length of the rail component.

27. The method of claim 23, wherein the composition is an adhesive composition.

28. The method of claim 23, wherein the composition is a bone restorative composition.

29. The method of claim 23, wherein each of the plurality of retaining elements grasps one of the two or more segments or structures.

30. The method of claim 23, wherein each of the plurality of retaining elements grasps a portion of the two or more bone segments or structures.

31. The method of claim 23, wherein each of the plurality of retaining elements comprises a plurality of opposing points or edges for grasping and holding the two or more segments or structures constructed and arranged to penetrate soft tissue and engage bone.

32. The method of claim 23, wherein each of the plurality of retaining elements comprises a surface constructed and arranged to support the two or more segments or structures.

33. The method of claim 32, wherein the surface comprises a cylindrical concave surface, conical concave surface, a flat surface, or a convex surface.

34. The method of claim 23, wherein the coupling between each of the one or more base elements and the plurality of retaining elements is constructed and arranged to be fixed into a position.

35. The method of claim 21, wherein positioning the two or more segments or structures comprises positioning the one or more base elements of the positioning device to limit the motion of the two or more segments or structures to one translational degree of freedom or rotational degree of freedom.

36. The method of claim 35, wherein the limiting of the motion of the two or more segments or structures is provided mechanically or chemically.

37. The method of claim 36, comprising limiting the motion of the two or more segments or structures by applying a compressive force to the plurality of retaining elements.

38. The method of claim 35, wherein positioning the two or more segments or structures comprises further comprises actuating a hinge operatively coupled to the plurality of retaining elements.

39. The method of claim 36, comprising limiting of the motion of the two or more segments or structures by application of a cyanoacrylate cement.

40. The method of claim 36, comprising limiting of the motion of the two or more segments or structures by crimping or swage deformation.

41. The method of claim 21, comprising maintaining the positioning of the two or more segments or structures using an applied force.

42. The method of claim 41, wherein the force applied is between 20 N to 150 N.

43. The method of claim 21, wherein the position of each of the plurality of base elements along the rail component is secured mechanically.

44. The method of claim 21, wherein each of the plurality of base elements of the positioning device further comprises a protrusion adapted to connect to an external support.

45. The method of claim 21, wherein the rail component of the positioning device further comprises one or more stops along its length arranged to limit translation of the plurality of base elements.

46. The method of claim 21, wherein each of the plurality of retaining elements of the positioning device comprise one or more contact points for maintaining contact with or grasping each of the two or more segments or structures.

47. The method of claim 21, further comprising handles having a length that are constructed and arranged to be connectable to each of the plurality of retaining elements.

48. The method of claim 46, wherein said handles are:
detachable from the rest of the positioning device;
to be detached once the segments or structures have been positioned and fixated; or
to reduce stress/strain on the segments or structures during the curing of the adhesive composition while the device is still attached to the bone segment or structure.

49. The method of claim 21, wherein repositioning the two or more segments or structures further comprises applying a compressive force to composition-bearing surfaces of the two or more segments or structures to be fixated.

50. A kit for positioning two or more segments or structures, comprising:
one or more devices or device components for positioning the two or more bone segments or structures; and
an adhesive composition to be applied to the two or more segments or structures.

51. The kit of claim 50, wherein the adhesive composition comprises solid components including a multivalent metal salt and an organic compound and liquid components including an aqueous medium.

52. The kit of claim 51, wherein the solid components and the liquid components of the adhesive composition are provided in separate packaging.

53. The kit of claim any one of claims 50-52, further comprising a device for application of the adhesive composition.

54. The kit of claim 53, wherein the device for the application of an adhesive composition is a syringe.
